SEQUENCE LISTING (38 sequences)

SEQ ID NO: 1 - DNA, 1797 bp, Solanum tuberosum
  source 1..1797: mol_type = genomic DNA
tacatattattttgtaggattataaaaaaggttacaacaatatatgtgtaagattatatcaatatttaatatatttcttatttatctatgtaaaatattgattgagaaatttgaatatgtatattttaatttaataaaaaataaaattaaaattacccaaatgcgaattgttttgttttataaaaaagtacttttctgaaaaattattttaaaaagtgtatctaaaaaaaagaaggtaatttccagtgacaattgcctaacaagctctaaaaaatcttagcataaattttgaatttttagtagaatttctatacccttggaggactttgttatgcaattttcttccctataaatggccctccatagctcaaatgagatatcagacaatttaaagaagtactattaacatttagaagatttctttctttcccaggtaaataaatcattttccctctttccttcttgctctttctttgtttatttgttcagatttttaccctttttgttttggttagattcattgacaatggcggaccttctttcaaactggtcaagcacattagaagcagttcctccaagtcattgcatcccagtgcatgaaagaccatcggatccagttgaaattgtggacaatattccagtcattgatttgggaaaagctaatggtgaagaacgaagtgttgttgttaaagaacttttgaaagcttttgaagaatatgggttttttcaggtttattatttatacaatagtacaactctgttcttttttctttttttttcttattgtatttaaaaatgatctgaaattgaaatgatgaaatagataatcaatcatggagtacccgtagatctaatggatgaagcaatgaaagtgtacaaagaatttttcagtctgccagcagcagagaaagcagaatatgcaaaggatgcagctaatgatacaaataggggtgcagctacactgtacagtagcagcgctaagcattatgattcagaggagcatcgttactggagagatgtcttggaacatagctgcaatcttgatgggaaagacaaaaaaacttggcctagtaaccctccaagatataggtacctacctaaactatgcttagcaaaattccctcttgttatttttcttacctagtatttgcttgtccttcagggaggttattggtgcatatggagatgaattgagaagggtgagcaaagttatcttgggtctgttagctgaagggctaggtttggaggcagggttctttgacacagaacttgggcagagaatgcttgtgaatcactatccagcatgcccagatccaagtttaaccttgggagttggtggacattgtgatcctaatctcataaccattatccaacaagaagtgtatggtcttcaaatattgaaggatgacaaatggattggtgtgcagcctatccgcaatgcatttgtggtcaattctggtttaccaattacggtaggtgtaacactttctcttaattttcatggtctacaagcgattctcttattgctctgttttttttgtataaatacaggtagttagcaatggaaagctaactagtgttgcacatcgtgtggtgacaaacacaactcattcacgaacctccattggtacttttatttgcccacacgatattgttgagcctgcaaaagcacttgttggtccggagaatcctccacagttcaaatcctttaattggggaattgattttatgccacattacctcagcaagaaatcagtttaccacgcatcattggagcccttcaaaatcgatgcttaa

SEQ ID NO: 2 - DNA, 6400 bp, Solanum tuberosum
  misc_feature 2167..2842: note = n is a, c, g, or t
  source 1..6400: mol_type = genomic DNA
ataacgtaaattttcaaaagagtggaggatgattgaaaaaaactcaaacatatcatcaaactttgagaaaatactcatttatgtcgttcgttaaaagtttggctcaatatttatgtcattatttgttaactgaaacgacatagatgaaccaaaattttaacagatgacatgaataaatcttttctcaaagttcgataacatatttgaacctttttccttttaaaaaataatttaattaatgacatggccgaaacataattgatcatatataaaaataaataaattagtttaccaataatgaaacctttatattaaccaaaatttggggggctaaatgattttcacgtgattgtattgtttttttattgcacatttatttgggaataataaagatttaactatatttgaaaggaaaaaggttaatatggtctttttacatatttggtcatcattattgttgccatatctttggatataaataaataaatgtagccatttagcacctccaatcaaattcacagtcatccaaattattctaagaaaaagagagagagagaacaatggatttctacaatttagccttattcttcatagctttagtaattgggattttcacattttatgctatattaatgagaattaatggttggtattatgcaatcaaattttgttcaaagaaatataacatccctcttggttatatgggtttgccatattttggcaacacactttcttacttcaaatctaccatttgtggtgatccaaattcattccttgatttctttgctactaggtaaattaactattttcattatcgtacttatttgctatgttgtttgaattcttgaaaaatattaatatgtacttgtcaaatcttttaaaaatagtgcatttttgaagaatctaacatgagtactgcaactgttattacaattacatttttgtagagtccaattgaacaaaatttttctttttttttttaaaggtttgggacaggaggaatgtatagggcatacatatttgggaagccaacaattatggtgacaaagccagaaataattagaaaagttttgatggatgaagaatatcttgaaagaggtttgcctaattatatgaaaaaattaattggattaacaacttcgattgaagaagataaatattttcgtcgattaacatctccagtaaaaagtcatggattattatccgattattttgattatatcgataaaactgtgagcactacattagagaaatacgctactacggaagaacctattgagtttctccataagatgcacaggcttgcatttgaggtgtttatgagacttcttattggtgatgaggttaatcaagaattttttgatcaaatgtttgtggagattactgctgtaattagtgctgttcacaacttgccaattaatctcccaggatttccttatcataagggactcaaggtaagatgtgttcaacttttaatattatttttatttcattttaaattttaagatataaatgtgttttttttaacttgccatcagtttatatttgtgtacttcaattttgagtgtgtataaacttgaattagtagacacatgacataatacacgtaggacatcatgtaggatgcaaattgtcacgaaggacgtgtgtgtttacttgttcaactttatataagtttaagtgtttacttattcaactttatataagtttaagtgtttacttgtgcgtgtccaaagttgaagggtacacgtgtgagatgaggccaagtaaaatggcatatttatgtattttagatagaataacatgttttttttttatatacataatatataaatgtgcccttcgatttggattcaactgacatatatgccctccaactttaggtgtgcacaaacagatgcttaaacatatataaaattgaacaaatagacacatttgtcttaagtggcgtacatgacaattttgtgtcctacgtgatgccctacatgtattatgttacgcaagacacgcaagacacgtgtgtctacttgtttaattttatacaagtttaagtgtctatttgtacacacccaaaattagtgggcatagatattagttgaagccaagttaaatgacatatttatgtattatatattttctacacatgtaatgtgggaccccccacccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntaccagcacccccccacccaaaaaaaataatttaagttcgtttttaaaaaatactttcaacttcaaaattttattttttcaccccaaccctcgacccccacctatcccgcccctaccagcccccaccccccaaaataatttaagtttatttttaaaaaatattttcagtttcaattcaaaaaatattttctactctctagtaagaataaaagatattttctcaaaaaaaaaattcattcataaatcaaacacataaaaatcttttttcgaaaaatattttatactcaccaaccaaacatgagaaaataagtccaaagtctacttattttctagaaaaacattttccttcctaccgaacacacccaaaatcacccctttcatcttttatcatgatatttgtggttgtaataaatataatatagagtgcaacattcatgtcaaagaccaaaaagtacgagatcactcttttcatttaatatttatcgtgttgtatatgtttattatgatttcaggctcgaaaagtactaggagggatatttcaaaaactaatagatgaaagaagagaagccatgaaggatggaaaatcaatgccaagggcaaacataattgatatgttgttatcaaacactaatcaagattatgaagccaatatattgagtgacaagaagatcgttgaaatcctagttttgttttcatttgctggttttgaacctgttgctcttatgtctgtcaaggcaatttttcacttgcaaaagcatccccatttcttggagaaagccaaagtaagtactctttattctgttttaggggtgtcatatgggtgagttaaactgaatttggacaggtggactgagttaattagtccaaaacttacttggactaaaatggactaacaaacgagtcataaactcaactcgtctaattagacaggttgagttaaatttggacgaattaaattgggctaagttaatatgtccaaaaatttggggctaaaatgagataacaaaaggatcataactcaactcgtctaattggtcgatccaaacctaagcgggttgggtgggtagtgtattcacgagtggatttgccacccctagtttattttattttatatgacgatatttgattaattattttttttaacaggaggaacaagaggaaatagtaaagagaagagcatcttcaaatgctggacttagttttgatgagattaggcaaatgacgtttgttagtaaggtaagacaatattatgatgttatatactttctataatagcatttttataaataataccattatacaaaaagtcatcagtacaaataattaaaaaaagaagatgaagtgataatacaaaaattggactaacatgcattattatttattaatgttatctcttttaatattgacaggtaattaatgaaacgttacgtattgctactgatcaaacggtattccttagagacacaagtactacttttaacataaatggttggtactttgccttattcttttgtatttatattattattttctatcgaaaaatcaaaatacgacaaatattttgaaacgacgaataattcattcattcatttatctcttgtatgtgtagggtacaccatacccaaagggtggaagttttttgcagttgtatggaatattcatatgaatcctgatgtttatgttcagcctaaggaatttaatccttcaagatgggatgtaagtgatatgcatcttaattaattgtgtttaagaaaaaaaaattcactttattttttttataatcaagaaatttttgagggttaataatgtatgattcaaaatatgttacgtaataagttgtgtttgccttttaccactttgaatattaggtattttatttgcagtagggtttaaactcatgacgagaatttatttatacataagaatattagtaaaattagctcataaaaatatgatttgttcaatgtcttatttattaattcaattaattttgactcatctaatttaattcatctaaaaaatagtgctaggataataataataataataataaaatcggaaatgtgctctaaactagaaccacgccctccactaggaagaaaaaaaattgattaacaatctcttaacattcttcaaacctaattttgaacctcggtatgttctcctattttgagttatagcctcgatgagttaaagacgaatcatgtcatatctagctaatcaattcttttcaaatcttcctcgatctatctctacctctccttaaacatcgttgtcaatcttttgtaccttaggagtaaaagataggatattagttgctttaccgttctcaatggtacatagctaaaaagtgtattatcatcaggacaatgcatgtgttcctccttttgagatatctgaactatctcaatttaactttttacgttacgaaaatcattcccacattacttattacttaatgatcaagtgaaattttgacaggatattgaaactaagccaggcatttttcttccattttcaatgggccccaaatcatgcccaggatccaatctggccaagcttcaaatttcagtaattcttcattattatcttcttcactacaggtaattaaatagcttcatacttataatatgaaaaaattaactaattatattttgaatattttatttttattttcttaccaaatattttcaaagggactaaaatatatgaatttattgtgaaatttataccaaaaaataagtattttgacccttatactcgtgattttatcgtatgaattgagatggatggaatatttaaaataaaaaaggaaatataaattattgattaaatcatattttaatttaattaaacgatatatttaatattcatgcagggttgagcaaattaatccagaggctagatgttatcctcctgaaaattgtcttgtgaaattcaagaagctctcaatctctagtgatggtaactaattttaatcattgtgttacaaataaataattcatgttgtaatcttgaaaacaataagtgtattgaataattattaaattggatatttttgtagaacgtttaatccagtttcaaatctagttggatacagagttacttttatcagaaaatgttacatcttctgattttatttttaaatgaaaaatattttgtatttccttaatagaaaaatcttaaattcatcagtgtttaaatgcatagtcaaacaaaccctaaattatatgaatttgatactaacaagtttgttttatctttcccaccacctgtatcgaatcggatcgaggatgataaaagaaaattataaactattttattaaaaatatatgaaatattttggacgggagggagcacagaacaccacttcattaaattttgtgacatttaattaatgtcggacttgaatcttgataatcacatcatattttatatttcgaattttatattaattgtttatttttttaaaaaaaattatttgttgcatagtgtattccacttgatctttcaatgtaaaagaggctctttattaaatttgatagcagagttatatta

SEQ ID NO: 3 - DNA, 2224 bp, Solanum tuberosum
  source 1..2224: mol_type = genomic DNA
ggatataaaaaagaagagttgaaattatatttctaattaaaatgaccatggaggtcaattagtcaaaaagttagatgatttttggatgacaacttagattagtgacgtttaacggtgaattagtataagttgagtgaccattcagacattcgactcgatatttagtcaattttacctttacatcagttgattttacaatgataaattagtagcaagaaaatggacagtcaatggttatattgtgcataatttccaacttttagtttcaagattatactgaacttggcgtttttcgccaaatgcttgttatatcatgttgctaatctgactttgaatagcattatgtttttggaagagcttgtttatttcaatagcattgagtttcttatatgaaaagtagtgctcaattggtatatgtgattgatggatgacaagtagtagtacatttcttggctgtggttcataacttggattggtcgttgtaacagttatacgtcctcaagtttccaagatgtcggatgctaaggcccccgcggccgccgttcaccctaggaggaagatccagttggtggactttcttctttcgttccgatggatcattgttatcttttttgtccttccattctcgttcttgtattacttctccatatatctaggggatttgaagtctgagaagaaatcttacaagcaacgccagatggaacacgatgagaatgttaaagaggttgtgaagcgccttgaacagaggaatgcagaaaaggatggtcttgtctgcacggccaggcctccttgggttgttgttggaatgagaaacgtagactataaacgtgctcgtcattttgaagttgatctttctaagtttagaaatatacttgatattgacacggaacgtatggttgccaaagttgagcctcttgtcaatatgggccaaatgtcaagggtcgccatcccaatgaatctttcccttgcagttcttgctgagcttgatgatcttaccgttggaggtttgatcaatgggttcggggtcgaaggaagttctcacatctttggattgttctctgacactgttgtagcacttgaggttgttctagctgatggaaaggttgttagagctacaaaggacaacgaatattctgatcttttctacgctattccgtggtctcaagggacattggggcttcttgtttcagctgaaatcaagcttataccagttgatcaatacgtaaaacttacctacaaacctgtaaggggtaatcttcaagagcttgcacaggcttacgcggattcttttgcacctaaagatggagatcaggacaatccttctaaagttccagagatggtagaaggcatgatttatggtccaacagaaggtgttatgatgaccggtatgtatgcttcgaggaatgaagccaaacgaaggggtaatgtaatcaacaattatggttggtggttcaaaccatggttttaccaacacgctcaaaccgcactgaaaagaggggaatttgtggagtacatcccaactagggactactaccacaggcacacgagatcgttgtattgggaagggaaactaattcttccatttggtgatcagttctggtttaggttcctcttaggatggctcatgccaccaaagattgctctgctcaaagccacacaaagtgaggctattagaaactattaccatgaccatcatgtcattcaggatctgcttgttcctctttacaaggtcggcgattgtctcgagtgggttcaccgcgagatggaggtatatcccatttggctctgcccacacagaatttacaagctgcctgtgagacctatgatctaccctgaaccaggattcgagaaacacaaaaggcagggtgacaccgaatatgcacaaatgtacactgatattggtgtctactatgttcccggagcagtcctgaggggtgagccatttgatggatcagagaaatgccgccaacttgagctttggttgattgaaaaccatggatttcaggctcaatacgcggtgactgagctgacagagaagaatttctggaggatgtttgataacagtctgtacgagcaatgcagaaggaagtacaaagctattggaacgttcatgagtgtgtactataaatcgaagaaaggaaggaagacagagaaggaagtgcaggaagctgagcaagagaaagctgaacaagagactcctgaagctgatgaacctgcgaattga

SEQ ID NO: 4 - DNA, 20 bp, synthetic construct
  misc_feature 1..20: note = Synthetic Polynucleotide
  source 1..20: mol_type = other DNA
gtaattcttcccctcaggag

SEQ ID NO: 5 - DNA, 20 bp, synthetic construct
  misc_feature 1..20: note = Synthetic Polynucleotide
  source 1..20: mol_type = other DNA
ccgatggtctttcatgcact

SEQ ID NO: 6 - DNA, 20 bp, synthetic construct
  misc_feature 1..20: note = Synthetic Polynucleotide
  source 1..20: mol_type = other DNA
cccagtgcatgaaagaccat

SEQ ID NO: 7 - DNA, 20 bp, synthetic construct
  misc_feature 1..20: note = Synthetic Polynucleotide
  source 1..20: mol_type = other DNA
tgcttcatccattagatcta

SEQ ID NO: 8 - DNA, 20 bp, synthetic construct
  misc_feature 1..20: note = Synthetic Polynucleotide
  source 1..20: mol_type = other DNA
tattaatgagaattaatggt

SEQ ID NO: 9 - DNA, 20 bp, synthetic construct
  misc_feature 1..20: note = Synthetic Polynucleotide
  source 1..20: mol_type = other DNA
tatatgggtttgccatattt

SEQ ID NO: 10 - DNA, 20 bp, synthetic construct
  misc_feature 1..20: note = Synthetic Polynucleotide
  source 1..20: mol_type = other DNA
tttggctttctccaagaaat

SEQ ID NO: 11 - DNA, 20 bp, synthetic construct
  misc_feature 1..20: note = Synthetic Polynucleotide
  source 1..20: mol_type = other DNA
ccctaggaggaagatccagt

SEQ ID NO: 12 - DNA, 20 bp, synthetic construct
  misc_feature 1..20: note = Synthetic Polynucleotide
  source 1..20: mol_type = other DNA
acgctattccgtggtctcaa

SEQ ID NO: 13 - DNA, 22 bp, synthetic construct
  misc_feature 1..22: note = Synthetic Polynucleotide
  source 1..22: mol_type = other DNA
ctatccttcgcaagacccttcc

SEQ ID NO: 14 - DNA, 24 bp, synthetic construct
  misc_feature 1..24: note = Synthetic Polynucleotide
  source 1..24: mol_type = other DNA
gtctgccagttcagttcgttgttc

SEQ ID NO: 15 - DNA, 25 bp, synthetic construct
  misc_feature 1..25: note = Synthetic Polynucleotide
  source 1..25: mol_type = other DNA
catttagaagatttctttctttccc

SEQ ID NO: 16 - DNA, 22 bp, synthetic construct
  misc_feature 1..22: note = Synthetic Polynucleotide
  source 1..22: mol_type = other DNA
gggtactccatgattgattatc

SEQ ID NO: 17 - DNA, 22 bp, synthetic construct
  misc_feature 1..22: note = Synthetic Polynucleotide
  source 1..22: mol_type = other DNA
gaagaatatgggttttttcagg

SEQ ID NO: 18 - DNA, 21 bp, synthetic construct
  misc_feature 1..21: note = Synthetic Polynucleotide
  source 1..21: mol_type = other DNA
tctccagtaacgatgctcctc

SEQ ID NO: 19 - DNA, 25 bp, synthetic construct
  misc_feature 1..25: note = Synthetic Polynucleotide
  source 1..25: mol_type = other DNA
ttgaaaggaaaaaggttaatatggt

SEQ ID NO: 20 - DNA, 22 bp, synthetic construct
  misc_feature 1..22: note = Synthetic Polynucleotide
  source 1..22: mol_type = other DNA
gctttgtcaccataattgttgg

SEQ ID NO: 21 - DNA, 25 bp, synthetic construct
  misc_feature 1..25: note = Synthetic Polynucleotide
  source 1..25: mol_type = other DNA
cgaaaagtactaggagggatatttc

SEQ ID NO: 22 - DNA, 25 bp, synthetic construct
  misc_feature 1..25: note = Synthetic Polynucleotide
  source 1..25: mol_type = other DNA
ctttactatttcctcttgttcctcc

SEQ ID NO: 23 - DNA, 27 bp, synthetic construct
  misc_feature 1..27: note = Synthetic Polynucleotide
  source 1..27: mol_type = other DNA
caaatgcttgttatatcatgttgctaa

SEQ ID NO: 24 - DNA, 20 bp, synthetic construct
  misc_feature 1..20: note = Synthetic Polynucleotide
  source 1..20: mol_type = other DNA
accatccttttctgcattcc

SEQ ID NO: 25 - DNA, 21 bp, synthetic construct
  misc_feature 1..21: note = Synthetic Polynucleotide
  source 1..21: mol_type = other DNA
ccaaagttgagcctcttgtca

SEQ ID NO: 26 - DNA, 23 bp, synthetic construct
  misc_feature 1..23: note = Synthetic Polynucleotide
  source 1..23: mol_type = other DNA
catctttaggtgcaaaagaatcc

SEQ ID NO: 27 - DNA, 9958 bp, synthetic construct
  variation 14replace
  variation 18replace
  variation 9871replace
  source 1..9958: mol_type = other DNA
ctgcactccacagnaaancaatcaaacaaacatacacagcgacttattcacacgagctcaaattacaacggtatatatcctgccagtcagcatcatcacaccaaaagttaggcccgaatagtttgaaattagaaagctcgcaattgaggtctccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattcgagctcggtaccggcgcgcctaactataacggtcctaaggtagcgatagggataacagggtaatgcggccgcgtcactggattttggttttaggaattagaaattttattgatagaagtattttacaaatacaaatacatactaagggtttcttatatgctcaacacatgagcgaaaccctataagaaccctaattcccttatctgggaactactcacacattattctggagaaaaatagagagagatagatttgtagagagagactggtgatttttgcggactctagatcaggtggatcccgggcccgcggtacaagaaagctgggtcggcgcgcccacccttcacaccttcctcttcttcttggggtcagccctgctgtctccaccgagctgagagaggtcgattcttgtttcatagagccccgtaattgactgatgaatcagtgtggcgtccaggacctcctttgtagaggtgtaccgctttctgtctatggtggtgtcgaagtacttgaaggctgcaggcgcgcccaagttggtcagagtaaacaagtggataatgttttctgcctgctccctgatgggcttatccctgtgcttattgtaagcagaaagcaccttatcgaggttagcgtcggcgaggatcactcttttggagaattcgcttatttgctcgatgatctcatcaaggtagtgtttgtgttgttccacgaacagctgcttctgctcattatcttcgggagaccctttgagcttttcatagtggctggccagatacaagaaattaacgtatttagagggcagtgccagctcgttacctttctgcagctcgcccgcactagcgagcattcgtttccggccgttttcaagctcaaagagagagtacttgggaagcttaatgatgaggtcttttttgacctctttatatcctttcgcctcgagaaagtcgatggggtttttttcgaagcttgatcgctccatgattgtgatgcccagcagttccttgacgcttttgagttttttagacttccctttctccactttggccacaaccagtacactgtaagcgactgtaggagaatcgaatccgccgtatttcttggggtcccaatcttttttgcgtgcgatcagcttgtcgctgttccttttcgggaggatactttccttggagaagcctccggtctgtacttcggtctttttaacgatgttcacctgcggcatggacaggaccttccggactgtcgcgaaatccctacccttgtcccacacgatttctcctgtttctccgtttgtttcgataagtggtcgcttccgaatctctccattggccagtgtaatctcggtcttgaaaaaattcataatattgctgtaaaagaagtacttagcggtggccttgcctatttcctgctcagactttgcgatcattttcctaacatcgtacactttatagtctccgtaaacaaattcagattcaagcttgggatattttttgataagtgcagtgcctaccactgcattcaggtaggcatcatgcgcatggtggtaattgttgatctctctcaccttataaaactgaaagtcctttctgaaatctgagaccagcttagacttcagagtaataactttcacctctcgaatcagtttgtcattttcatcgtacttggtgttcatgcgtgaatcgagaatttgggccacgtgcttggtgatctggcgtgtctcaacaagctgccttttgatgaagccggctttatccaactcagacaggccacctcgttcagccttagtcagattatcgaacttccgttgtgtgatcagtttggcgttcagcagctgccgccaataatttttcattttcttgacaacttcttctgaggggacgttatcactcttccctctatttttatcggatcttgtcaacactttattatcaatagaatcatctttgagaaaagactggggcacgatatgatccacgtcgtagtcggagagccgattgatgtccagttcctgatccacgtacatgtccctgccgttctgcaggtagtacaggtagagcttctcattctgaagctgggtgttttcaactgggtgttccttaaggatttgggaccccagttcttttataccctcttcaatcctcttcatcctttccctactgttcttctgtcccttctgggtagtttggttctctcgggccatctcgataacgatattctcgggcttatgccttcccattactttgacgagttcatccacgaccttaacggtctgcagtattccctttttgatagctgggctacctgcaagattagcgatgtgctcgtgaagactgtccccctggccagaaacttgtgctttctggatgtcctccttaaaggtgagagagtcatcatggatcaactgcatgaagttccggttggcaaatccatcggacttaagaaaatccaggattgtctttccactctgcttgtctcggatcccattgatcagttttcttgacagccgcccccatcctgtatatcggcgcctcttgagctgtttcatgactttgtcgtcgaagagatgagcgtaagttttcaagcgttcttcaatcatctccctatcttcaaacaacgtaagggtgaggacaatgtcctcaagaatgtcctcgttctcctcattgtccaggaagtccttgtctttaatgattttcaggagatcgtgatacgttcccagggatgcgttgaagcgatcctccactccgctgatttcaacagagtcgaaacattcaatctttttgaaatagtcttctttgagctgtttcacggtaactttccggttcgtcttgaagaggaggtccacgatagctttcttctgctctccagacaggaatgctggctttctcatcccttctgtgacgtatttgaccttggtgagctcgttataaactgtgaagtactcgtacagcagagagtgtttaggaagcaccttttcgttaggcagatttttatcaaagttagtcatcctttcgatgaaggactgggcagaggcccccttatccacgacttcctcgaagttccagggagtgatggtctcttctgatttgcgagtcatccacgcgaatctggaatttccccgggcgagggggcctacatagtagggtatccgaaatgtgaggattttctcaatcttttccctgttatctttcaaaaaggggtagaaatcctcttgccgcctgaggatagcgtgcagttcgcccaggtgaatctggtgggggatgcttccattgtcgaaagtgcgctgtttgcgcaacagatcttctctgttaagctttaccagcagctcctcggtgccgtccattttttccaagatgggcttaataaatttgtaaaattcctcctggcttgctccgccgtcaatgtatccggcgtagccatttttagactgatcgaagaaaatttccttgtacttctcaggcagttgctgtctgacaagggccttcagcaaagtcaagtcttggtggtgctcatcatagcgcttgatcatactagcgctcagcggagctttggtgatctccgtgttcactcgcagaatatcactcagcagaatggcgtctgacaggttctttgccgccaaaaaaaggtctgcgtactggtcgccgatctgggccagcagattgtcgagatcatcatcgtaggtgtctttgctcagttgaagcttggcatcttcggccaggtcgaagttagatttaaagttgggggtcagcccgagtgacagggcgataagattaccaaacaggccgttcttcttctccccagggagctgtgcgatgaggttttcgagccgccgggatttggacagcctagcgctcaggattgctttggcgtcaactccggatgcgttgatcgggttctcttcgaaaagctgattgtaagtctgaaccagttggataaagagtttgtcgacatcgctgttgtctgggttcaggtccccctcgatgaggaagtgtccccgaaatttgatcatatgcgccagcgcgagatagatcaaccgcaagtcagccttatcagtactgtctacaagcttcttcctcagatgatatatggttgggtacttttcatggtacgccacctcgtccacgatattgccaaagattgggtggcgctcgtgctttttatcctcctccaccaaaaaggactcctccagcctatggaagaaagagtcatccaccttagccatctcattactaaagatctcctgcaggtagcagatccgattctttctgcgggtatatctgcgccgtgctgttcttttgagccgcgtggcttcggccgtctccccggagtcgaacaggagggcgccaatgaggttcttctttatgctgtggcgatcggtattgcccagaactttgaattttttgctcggcaccttgtactcgtccgtaatgacggcccagccgacgctgtttgtgccgatatcgagcccaatggagtacttcttgtccatggaatcagaaaaactcagattaatcgacaaattcgatcgcacaaactagaaactaacaccagatctagatagaaatcacaaatcgaagagtaattattcgacaaaactcaaattatttgaacaaatcggatgatatctatgaaaccctaatcgagaattaagatgatatctaacgatcaaacccagaaaatcgtcttcgatctaagattaacagaatctaaaccaaagaacatatacgaaattgggatcgaacgaaaacaaaatcgaagattttgagagaataaggaacacagaaatttaccttgatcacggtagagagaattgagagaaagtttttaagattttgagaaattgaaatctgaattgtgaagaagaagagctctttgggtattgttttatagaagaagaagaagaaaagacgaggacgactaggtcacgagaaagctaaggcggtgaagcaatagctaataataaaatgacacgtgtattgagcgttgtttacacgcaaagttgtttttggctaattgccttatttttaggttgaggaaaagtatttgtgctttgagttgataaacacgactcgtgtgtgccggctgcaaccactttgacgccgtttattactgactcgtcgacaaccacaatttctaacggtcgtcataagatccagccgttgagatttaacgatcgttacgatttatatttttttagcattatcgttttattttttaaatatacggtggagctgaaaattggcaataattgaaccgtgggtcccactgcattgaagcgtatttcgtatcttctagaattcttcgtgctttatttcttttcctttttgtttttttttgccatttatctaatgcaagtgggcttataaaatcagtgaatttcttggaaaagtaacttctttatcgtataacatattgtgaaattatccatttcttttaatttttttagtgttattggatatttttgtatgattattgatttgcataggataatgacttttgtatcaagttggtgaacaagtctcgttaaaaaaggcaagtggtttggtgactcgatttattcttgttatttaattcatatatcaatggatcttatttggggcctggtccatatttaacactcgtgttcagtccaatgaccaataatattttttcattaataacaatgtaacaagaatgatacacaaaacattctttgaataagttcgctatgaagaagggaacttatccggtcctagatcatcagttcatacaaacctccatagagttcaacatcttaaacaagaatatcctgatctgaagaatgtggaggctttagtcccttggatacttgggaggctgtggaagaacagaaacgagctggtgcttaaagggagggaatttggaaccaatgaggtattagtaaggacacaagaagatgcagatgagtggattagaaggaaagaggctcagaaccggttagggataacagggtaattggcaaacagctattatgggtattatgggtggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgcaagctcctcgaggagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatccgtcctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctctattttctccataataatgtgtgagtagttcccagataagggaattagggttcttatagggtttcgctcagatccgtcgacgtcgaggaattccccggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactscaggacgaggcagcgcggctatcgtggctggccacgacgggcgttccttgcgcagctgtsctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccggaattcccgggggaggaaattacactgaggaaggagaagatgacgacgatgagatggacgatgaaggggaggctggtggagcggaaccaagagagtgtcagatcggaaaccttatcaattatccgatcattgctttagggtcatgcgatctttccgcataattcccgtcgccgacacctaataaagtcggctaatctatgtgattgagtgtgtcttgactttgttattttgcatgtttccaatgtcatttagtaacgaaataaacgttatcctcttctaaaagcaggctgtgttttcggcaaacatcgccacccatcgctagtttttctaaaagtgttctaagctagcctggtaataatctatacgagcttatatttctaatcattgccgaaaaatcctgtttcgaaataattttgtaactctctttaatatcaccacgatcacacaagaagaagaattaaatataacatttatcagcccacgatgaacatggcgaaaattacaagcgaacacaattgtcttattcattaataattaattcaacaaagttctcagacactccttgatttaacagaagattttataagcacctctatgatgcaacaaccaaaacgacatagacttcgaaatcccgccccataaaaactagatagcaccatctaactccacgtcttcaagagcagcctccgggaggaaagctgaacagcccacagcgatcaaaagataatccaatggttggccggcgcttggcattttccttttccaaacgtccttgctttgaccaaaagtgttccggacgactcctatgcgttcgaactcgttgctggcctgaattgctagaaagtacgaggatgaatattcatttttgctgtagttgatccgaccaccgtctccgcttaaggcttgcagttgcgccctcacctgccggaatgtataaccatcagaatagggttccagtgtcgtggcaagcaatccatctgaagcatttgaaactagaaccattctttgccgacataaccgataattctctttatcgagataaatgtacagaaattccccgccaggattgatgatgtcgccagacttagtgtgtagatcggtttgaaacttttcccgataaacttttgaaagctccgagagttcaccccctcgaggcgatcgtgaagtttctcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcccggacatgaagccatttacaattgaatatatcctgccgccgctgccgctttgcacccggtggagcttgcatgttggtttctacgcagaactgagccggttaggcagataatttccattgagaactganccatgtgcaccttccccccaacacggtgagcgacgggcaacggagtgatccacatgggacttttaaacatcatccgtcgatgcgttg

SEQ ID NO: 28 - DNA, 5430 bp, synthetic construct
  misc_feature 1..5430: note = DNA Construct
  source 1..5430: mol_type = other DNA
tggctggtggcaggatatattgtggtgtaaacaaattgacgcttagacaacttaataacacattgcggacgtttttaatgtactgaattaacgccgaattgaattcctcgagtacgtaggatccatttaaattctagaggcgcgccgcggccgctcctgctgagcctcgacatgttgtcgcaaaattcgccctggacccgcccaacgatttgtcgtcactgtcaaggtttgacctgcacttcatttggggcccacatacaccaaaaaaatgctgcataattctcggggcagcaagtcggttacccggccgccgtgctggaccgggttgaatggtgcccgtaactttcggtagagcggacggccaatactcaacttcaaggaatctcacccatgcgcgccggcggggaaccggagttcccttcagtgagcgttattagttcgccgctcggtgtgtcgtagatactagcccctggggcacttttgaaatttgaataagatttatgtaatcagtcttttaggtttgaccggttctgccgctttttttaaaattggatttgtaataataaaacgcaattgtttgttattgtggcgctctatcatagatgtcgctataaacctattcagcacaatatattgttttcattttaatattgtacatataagtagtagggtacaatcagtaaattgaacggagaatattattcataaaaatacgatagtaacgggtgatatattcattagaatgaaccgaaaccggcggtaaggatctgagctacacatgctcaggttttttacaacgtgcacaacagaattgaaagcaaatatcatgcgatcataggcgtctcgcatatctcattaaagcaggactctagggactagttctagactaggtggatcctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgtcgactgcagaattcgaagcttgagctcgagatctgagtccggattggtagattgcaaatataatggtttggcgattatccttgaggccacacctttaaatagatcaatgaatgggcaagtattgacttgcgataatcctttaactttctgcctaccatcaatgtggatgagttgtcggtaaaaaggatcttacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagcccggtggcgcgccgatatcctctcttaaggtagcgagctcttaattaatagggataacagggtaattaactataacggtcctaaggtagcgatggcaaacagctattatgggtattatgggtgcggccgcgtcactggattttggttttaggaattagaaattttattgatagaagtattttacaaatacaaatacatactaagggtttcttatatgctcaacacatgagcgaaaccctataagaaccctaattcccttatctgggaactactcacacattattctggagaaaaatagagagagatagatttgtagagagagactggtgatttttgcggactctagatcaggtggatcctcattgtttgcctccctgctgcggtttttcaccgaagttcatgccagtccagcgtttttgcagcagaaaagccgccgacttcggtttgcggtcgcgagtgaagatccctttcttgttaccgccaacgcgcaatatgccttgcgaggtcgcaaaatcggcgaaattccatacctgttcaccgacgacggcgctgacgcgatcaaagacgcggtgatacatatccagccatgcacactgatactcttcactccacatgtcggtgtacattgagtgcagcccggctaacgtatccacgccgtattcggtgatgataatcggctgatgcagtttctcctgccaggccagaagttctttttccagtaccttctctgccgtttccaaatcgccgctttggacataccatccgtaataacggttcaggcacagcacatcaaagagatcgctgatggtatcggtgtgagcgtcgcagaacattacattgacgcaggtgatcggacgcgtcgggtcgagtttacgcgttgcttccgccagtggcgcgaaatattcccgtgcaccttgcggacgggtatccggttcgttggcaatactccacatcaccacgcttgggtggtttttgtcacgcgctatcagctctttaatcgcctgtaagtgcgcttgctgagtttccccgttgactgcctcttcgctgtacagttctttcggcttgttgcccgcttcgaaaccaatgcctaaagagaggttaaagccgacagcagcagtttcatcaatcaccacgatgccatgttcatctgcccagtcgagcatctcttcagcgtaagggtaatgcgaggtacggtaggagttggccccaatccagtccattaatgcgtggtcgtgcaccatcagcacgttatcgaatcctttgccacgcaagtccgcatcttcatgacgaccaaagccagtaaagtagaacggtttgtggttaatcaggaactgttcgcccttcactgccactgaccggatgccgacgcgaagcgggtagatatcacactctgtctggcttttggctgtgacgcacagttcatagagataaccttcacccggttgccagaggtgcggattcaccacttgcaaagtcccgctagtgccttgtccagttgcaaccacctgttgatccgcatcacgcagttcaacgctgacatcaccattggccaccacctgccagtcaacagacgcgtggttacagtcttgcgcgacatgcgtcaccacggtgatatcgtccacccaggtgttcggcgtggtgtagagcattacgctgcgatggattccggcatagttaaagaaatcatggaagtaagactgctttttcttgccgttttcgtcggtaatcaccattcccggcgggatagtctgccagttcagttcgttgttcacacaaacggtgatacgtacacttttcccggcaataacatacggcgtgacatcggcttcaaatggcgtatagccgccctgatgctccatcacttcctgattattgacccacactttgccgtaatgagtgaccgcatcgaaacgcagcacgatacgctggcctgcccaacctttcggtataaagacttcgcgctgataccagacgttgcccgcataattacgaatatctgcatcggcgaactgatcgttaaaactgcctggcacagcaattgcccggctttcttgtaacgcgctttcccaccaacgctgatcaattccacagttttcgcgatccagactgaatgcccacaggccgtcgagttttttgatttcacgggttggggtttctacaggacgtaattcttcccctcaggaggggaagaacatggtaccgtcgactgcagaattcgaagcttgagctcgagatctctatcgttcgtaaatggtgaaaattttcagaaaattgcttttgctttaaaagaaatgatttaaattgctgcaatagaagtagaatgcttgattgcttgagattcgtttgttttgtatatgttgtgttgagaactctcgacgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccacatgttgaccggttggcaaacagctattatgggtattatgggtggttctttatgcggacactgacggctttatgcctgcaggtcgcgagcgatcgcggtaccgcccgggcgtcgacaggcctaagcttagcttgagcttggatcagattgtcgtttcccgccttcagtttaaactatcagtgtttgacaggatatattggcgggtaaacctaaga

SEQ ID NO: 29 - DNA, 19370 bp, synthetic construct
  misc_feature 1..19370: note = DNA Construct
  source 1..19370: mol_type = other DNA
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaagctagcttgcatgcctgcaggtcactattccagtatggacgattcaaggcttgcttcataaaccaaggcaagtaatagagattggagtctctaagaaagtagttcctactgaatcaaaggccatggagtcaaaaattcagatcgaggatctaacagaactcgccgtgaagactggcgaacagttcatacagagtcttttacgactcaatgacaagaagaaaatcttcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatcggatccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgatctcacgcggactctagaggatcccccgatgagctaagctagctatatcatcaatttatgtattacacataatatcgcactcagtctttcatctacggcaatgtaccagctgatataatcagttattgaaatatttctgaatttaaactttgcatcaataaattatgtttttttgcttggactataatacctgacttattttatcaataaatatttaaactatatttctttcaagatgggaattcatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcatgggacgtcgacctgaggtaattataacccgggccctatatatggatccaattgcaatgatcatcatgacagatctgcgcgcgatcgatatcagcgctttaaatttgcgcatgcatgcctgcaggtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatggtcaacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggataaaacatttcaatcctttgaacgcggtagaacgtgctaattggattttggtgagaacgcggtagaacgtacttatcacctacagttttattttgtttttctttttggtttaatctatccagcttagtaccgagtgggggaaagtgactggtgtgcctaaaaccttttctttgatactttgtaaaaatacatacagatacaatggcgaacggtaacttcaagttgtctcaattgctcaatgtggacgagatgtctgctgagcagaggagtcatttctttgacttgatgctgactaaacctgattgtgagatcgggcaaatgatgcaaagagttgttgttgataaagtcgatgacatgattagagaaagaaagactaaagatccagtgattgttcatgaagttctttctcagaaggaacagaacaagttgatggaaatttatcctgaattcaatatcgtgtttaaagacgacaaaaacatggttcatgggtttgcggctgctgagcgaaaactacaagctttattgcttttagatagagttcctgctctgcaagaggtggatgacatcggtggtcaatggtcgttttgggtaactagaggtgagaaaaggattcattcctgttgtccaaatctagatattcgggatgatcagagagaaatttctcgacagatatttcttactgctattggtgatcaagctagaagtggtaagagacagatgtcggagaatgagctgtggatgtatgaccaatttcgtgaaaatattgctgcgcctaacgcggttaggtgcaataatacatatcagggttgtacatgtaggggtttttctgatggtaagaagaaaggcgcgcagtatgcgatagctcttcacagcctgtatgacttcaagttgaaagacttgatggctactatggttgagaagaaaactaaagtggttcatgctgctatgctttttgctcctgaaagtatgttagtggacgaaggtccattaccttctgttgacggttactacatgaagaagaacgggaagatctatttcggttttgagaaagatccttccttttcttacattcatgactgggaagagtacaagaagtatctactggggaagccagtgagttaccaagggaatgtgttctacttcgaaccgtggcaggtgagaggagacacaatgcttttttcgatctacaggatagctggagttccgaggaggtctctatcatcgcaagagtactaccgaagaatatatatcagtagatgggaaaacatggttgttgtcccaattttcgatctggtcgaatcaacgcgagagttggtcaagaaagacctgtttgtagagaaacaattcatggacaagtgtttggattacatagctaggttatctgaccagcagctgaccataagcaatgttaaatcatacttgagttcaaataattgggtcttattcataaacggggcggccgtgaagaacaagcaaagtgtagattctcgagatttacagttgttggctcaaactttgctagtgaaggaacaagtggcgagacctgtcatgagggagttgcgtgaagcaattctgactgagacgaaacctatcacgtcattgactgatgtgctgggtttaatatcaagaaaactgtggaagcagtttgctaacaagatcgcagtcggcggattcgttggcatggttggtactctaattggattctatccaaagaaggtactaacctgggcgaaggacacaccaaatggtccagaactatgttacgagaactcgcacaaaaccaaggtgatagtatttctgagtgttgtgtatgccattggaggaatcacgcttatgcgtcgagacatccgagatggactggtgaaaaaactatgtgatatgtttgatatcaaacggggggcccatgtcttagacgttgagaatccgtgccgctattatgaaatcaacgatttctttagcagtctgtattcggcatctgagtccggtgagaccgttttaccagatttatccgaggtaaaagccaagtctgataagctattgcagcagaagaaagaaatcgctgacgagtttctaagtgcaaaattctctaactattctggcagttcggtgagaacttctccaccatcggtggtcggttcatctcgaagcggactgggtctgttgttggaagacagtaacgtgctgacccaagctagagttggagtttcaagaaaggtagacgatgaggagatcatggagcagtttctgagtggtcttattgacactgaagcagaaattgacgaggttgttccagccttttcagctgaatgtgaaagaggggaaacaagcggtacaaaggtgttgtgtaaacctttaacgccaccaggatttgagaacgtgttgccagctgtcaaacctttggtcagcaaaggaaaaacggtcaaacgtgtcgattacttccaagtgatgggaggtgagagattaccaaaaaggccggttgtcagtggagacgattctgtggacgctagaagagagtttctgtactacttagatgcggagagagtcgctcaaaatgatgaaattatgtctctgtatcgtgactattcgagaggagttattcgaactggaggtcagaattacccgcacggactgggagtgtgggatgtggagatgaagaactggtgcatacgtccagtggtcactgaacatgcttatgtgttccaaccagacaaacgtatggatgattggtcgggatacttagaagtggctgtttgggaacgaggtatgttggtcaacgacttcgcggtcgaaaggatgagtgattatgtcatagtttgcgatcagacgtatctttgcaataacaggttgatcttggacaatttaagtgccctggatctaggaccagttaactgttcttttgaattagttgacggtgtacctggttgtggtaagtcgacaatgattgtcaactcagctaatccttgtgtcgatgtggttctctctactgggagagcagcaaccgacgacttgatcgagagattcgcgagcaaaggttttccatgcaaattgaaaaggagagtgaagacggttgattcttttttgatgcattgtgttgatggttctttaaccggagacgtgttgcatttcgatgaagctctcatggcccatgctggtatggtgtacttttgcgctcagatagctggtgctaaacgatgtatctgtcaaggagatcagaatcaaatttctttcaagcctagggtatctcaagttgatttgaggttttctagtctggtcggaaagtttgacattgttacagaaaaaagagaaacttacagaagtccagcagatgtggctgccgtattgaacaagtactatactggagatgtcagaacacataacgcgactgctaattcgatgacggtgaggaagattgtgtctaaagaacaggtttctttgaagcctggtgctcagtacataactttccttcagtctgagaagaaggagttggtaaatttgttggcattgaggaaagtggcagctaaagtgagtacagtacacgagtcgcaaggagagacattcaaagatgtagtcctagtcaggacgaaacctacggatgactcaatcgctagaggtcgggagtacttaatcgtggcgttgtcgcgtcacacacaatcacttgtgtatgaaactgtgaaagaggacgatgtaagcaaagagatcagggaaagtgccgcgcttacgaaggcggctttggcaagattttttgttactgagaccgtcttatgacggtttcggtctaggtttgatgtctttagacatcatgaagggccttgcgccgttccagattcaggtacgattacggacttggagatgtggtacgacgctttgtttccgggaaattcgttaagagactcaagcctagacgggtatttggtggcaacgactgattgcaatttgcgattagacaatgttacgatcaaaagtggaaactggaaagacaagtttgctgaaaaagaaacgtttctgaaaccggttattcgtactgctatgcctgacaaaaggaagactactcagttggagagtttgttagcattgcagaaaaggaaccaagcggcacccgatctacaagaaaatgtgcacgcaacagttctaatcgaagagacgatgaagaagttgaaatctgttgtctacgatgtgggaaaaattcgggctgatcctattgtcaatagagctcaaatggagagatggtggagaaatcaaagcacagcggtacaggctaaggtagtagcagatgtgagagagttacatgaaatagactattcgtcttacatgtatatgatcaaatctgacgtgaaacctaagactgatttaacaccgcaatttgaatactcagctctacagactgttgtgtatcacgagaagttgatcaactcgttgttcggtccaattttcaaagaaattaatgaacgcaagttggatgctatgcaaccacattttgtgttcaacacgagaatgacatcgagtgatttaaacgatcgagtgaagttcttaaatacggaagcggcttacgactttgttgagatagacatgtctaaattcgacaagtcggcaaatcgcttccatttacaactgcagctggagatttacaggttatttgggctagatgagtgggcggccttcctttgggaggtgtcgcacactcaaactactgtgagagatattcaaaatggtatgatggcgcatatttggtaccaacaaaagagtggagatgctgatacttataatgcaaattcagatagaacactgtgtgcactcttgtctgaattaccattggagaaagcagtcatggttacatatggaggagatgactcactgattgcgtttcctagaggaacgcagtttgttgatccgtgtccaaagttggctactaagtggaatttcgagtgcaagatttttaagtacgatgtcccaatgttttgtgggaagttcttgcttaagacgtcatcgtgttacgagttcgtgccagatccggtaaaagttctgacgaagttggggaaaaagagtataaaggatgtgcaacatttagccgagatctacatctcgctgaatgattccaatagagctcttgggaactacatggtggtatccaaactgtccgagtctgtttcagaccggtatttgtacaaaggtgattctgttcatgcgctttgtgcgctatggaagcatattaagagttttacagctctgtgtacattattccgagacgaaaacgataaggaattgaacccggctaaggttgattggaagaaggcacagagagctgtgtcaaacttttacgactggtaatatggaagacaagtcattggtcaccttgaagaagaagactttcgaagtctcaaaattctcaaatctaggggccattgaattgtttgtggacggtaggaggaagagaccgaagtattttcacagaagaagagaaactgtcctaaatcatgttggtgggaagaagagtgaacacaagttagacgtttttgaccaaagggattacaaaatgattaaatcttacgcgtttctaaagatagtaggtgtacaactagttgtaacatcacatctacctgcagatacgcctgggttcattcaaatcgatctgttggattcgagacttactgagaaaagaaagagaggaaagactattcagagattcaaagctcgagcttgcgataactgttcagttgcgcagtacaaggttgaatacagtatttccacacaggagaacgtacttgatgtctggaaggtgggttgtatttctgagggcgttccggtctgtgacggtacataccctttcagtatcgaagtgtcgctaatatgggttgctactgattcgactaggcgcctcaatgtggaagaactgaacagttcggattacattgaaggcgattttaccgatcaagaggttttcggtgagttcatgtctttgaaacaagtggagatgaagacgattgaggcgaagtacgatggtccttacagaccagctactactagacctaagtcattattgtcaagtgaagatgttaagagagcgtctaataagaaaaactcgtcttaatgcataaagaaatttattgtcaatatgacgtgtgtactcaagggttgtgtgaatgaagtcactgttcttggtcacgagacgtgtagtatcggtcatgctaacaaattgcgaaagcaagttgctgacatggttggtgtcacacgtaggtgtgcggaaaataattgtggatggtttgtctgtgttgttatcaatgattttacttttgatgtgtataattgttgtggccgtagtcaccttgaaaagtgtcgtaaacgtgttgaaacaagaaatcgagaaatttggaaacaaattcgacgaaatcaagctgaaaacatgtctgcgacagctaaaaagtctcataattcgaagacctctaagaagaaattcaaagaggacagagaatttgggacaccaaaaagatttttaagagatgatgttcctttcgggattgatcgtttgtttgctttttgattttattttatattgttatctgtttctgtgtatagactgtttgagattggcgcttggccgactcattgtcttaccataggggaacggactttgtttgtgttgttattttatttgtattttattaaaattctcaatgatctgaaaaggcctcgaggctaagagattattggggggtgagtaagtacttttaaagtgatgatggttacaaaggcaaaaggggtaaaacccctcgcctacgtaagcgttattacgcccgtctgtacttatatcagtacactgacgagtccctaaaggacgaaacgggcccctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatatcaactcccctatccattgctcaccgaatggtacaggtcggggacccgaagttccgactgtcggcctgatgcatccccggctgatcgaccccagatctggggctgagaaagcccagtaaggaaacaactgtaggttcgagtcgcgagatcccccggaaccaaaggaagtaggttaaacccgctccgatcaggccgagccacgccaggccgagaacattggttcctgtaggcatcgggattggcggatcaaacactaaagctactggaacgagcagaagtcctccggccgccagttgccaggcggtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgaccccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgggtgatgctgccaacttactgatttagtgtatgatggtgtttttgaggtgctccagtggcttctgtgtctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgctgtcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacccaattatgatagaatttacaagctataaggttattgtcctgggtttcaagcattagtccatgcaagtttttatgctttgcccattctatagatatattgataagcgcgctgcctatgccttgccccctgaaatccttacatacggcgatatcttctatataaaagatatattatcttatcagtattgtcaatatattcaaggcaatctgcctcctcatcctcttcatcctcttcgtcttggtagctttttaaatatggcgcttcatagagtaattctgtaaaggtccaattctcgttttcatacctcggtataatcttacctatcacctcaaatggttcgctgggtttatcgcacccccgaacacgagcacggcacccgcgaccactatgccaagaatgcccaaggtaaaaattgccggccccgccatgaagtccgtgaatgccccgacggccgaagtgaagggcaggccgccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcgcaaaggcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccagatctggggaaccctgtggttggcatgcacatacaaatggacgaacggataaaccttttcacgcccttttaaatatccgattattctaataaacgctcttttctcttag

SEQ ID NO: 30 - DNA, 9702 bp, synthetic construct
  misc_feature 1..9702: note = DNA Construct
  source 1..9702: mol_type = other DNA
cattgcacctatggtgttgccctggctggggtatgtcagtgatcgcagtagaatgtactaattgacaagttggagaatacggtagaacgtccttatccaacacagcctttatccctctccctgacgaggtttttgtcagtgtaatatttctttttgaactatccagcttagtaccgtacgggaaagtgactggtgtgcttatctttgaaatgttactttgggtttcggttctttaggttagtaagaaagcacttgtcttctcatacaaaggaaaacctgagacgtatcgcttacgaaagtagcaatgaaagaaaggtggtggttttaatcgctaccgcaaaaacgatggggtcgttttaattaacttctcctacgcaagcgtctaaacggacgttggggttttgctagtttctttagagaaaactagctaagtctttaatgttatcattagagatggcataaatataatacttgtgtctgctgataagatcattttaatttggacgattagacttgttgaactacaggttactgaatcacttgcgctaatcaacaagctatcaagcttgttaacgaggtaattcttcccctcaggaggttttagagctagaaatagcaagttaaaataaggctagtccgttatcaacttgaaaaagtggcaccgagtcggtgctttttttctcgagggatttaaggacgtgaactctgttgagatctctgtgaaattcagagggtgggtgataccatattcactgatgccattagcgacatctaaatagggctaattgtgactaatttgagggaatttcctttaccattgacgtcagtgtcgttggtagcatttgagtttcgtaactataacggtcctaaggtagcgaacatcttgttctggggtttcacactatctttagagaaagtgttaagttaattaagttatcttaattaagagcataattatactgatttgtctctcgttgatagagtctatcattctgttactaaaaatttgacaactcggtttgctgacctactggttactgtatcacttacccgagttaacgagatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtgagtcgaccgggcatgtcccgaagacattaaactacggttctttaagtagatccgtgtctgaagttttaggttcaatttaaacctacgagattgacattctcgactgatcttgattgatcggtaagtcttttgtaatttaattttctttttgattttattttaaattgttatctgtttctgtgtatagactgtttgagatcggcgtttggccgactcattgtcttaccataggggaacggactttgtttgtgttgttattttatttgtattttattaaaattctcaacgatctgaaaaagcctcgcggctaagagattgttggggggtgagtaagtacttttaaagtgatgatggttacaaaggcaaaaggggtaaaacccctcgcctacgtaagcgttattacgcccgtctgtacttatatcagtacactgacgagtccctaaaggacgaaacgggagaacgctagccaccaccaccaccaccacgtgtgaattacaggtgaccagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcctaggccaccatgttgggcccggcgcgccatcaacatgtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggatccggcgcgccataaaa

SEQ ID NO: 31 - DNA, 6347 bp, synthetic construct
  misc_feature 1..6347: note = DNA Construct
  source 1..6347: mol_type = other DNA
ccatcatcagttcggtggtcttccgacgaacaataaggccgcaaatcgcggccttttttattgataacaaaaccggctcagttctgcgtagaaaccaacatgcaagctccaccgggtgcaaagcggcagcggcggcaggatatattcaattgtaaatggcttcatgtccgggaaatctactggtggcaggatatattgtggtgtaaacaatggagaaaaagattaattaagtactagtagcctggtcgaaaccgtctcaggagccggggatcccccctcagaagaccagagggctattgagacttttcaacaaagggtaatatcgggaaacctcctcggattccattgcccagctatctgtcacttcatcgaaaggacagtagaaaaggaaggtggctcctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggatccggcgcgccataaaacattgcacctatggtgttgccctggctggggtatgtcagtgatcgcagtagaatgtactaattgacaagttggagaatacggtagaacgtccttatccaacacagcctttatccctctccctgacgaggtttttgtcagtgtaatatttctttttgaactatccagcttagtaccgtacgggaaagtgactggtgtgcttatctttgaaatgttactttgggtttcggttctttaggttagtaagaaagcacttgtcttctcatacaaaggaaaacctgagacgtatcgcttacgaaagtagcaatgaaagaaaggtggtggttttaatcgctaccgcaaaaacgatggggtcgttttaattaacttctcctacgcaagcgtctaaacggacgttggggttttgctagtttctttagagaaaactagctaagtctttaatgttatcattagagatggcataaatataatacttgtgtctgctgataagatcattttaatttggacgattagacttgttgaactacaggttactgaatcacttgcgctaatcaacaagcttccttcccagtgcatgaaagaccatgttttagagctagaaatagcaagttaaaataaggctagtccgttatcaacttgaaaaagtggcaccgagtcggtgctttttttcctaagggatttaaggacgtgaactctgttgagatctctgtgaaattcagagggtgggtgataccatattcactgatgccattagcgacatctaaatagggctaattgtgactaatttgagggaatttcctttaccattgacgtcagtgtcgttggtagcatttgagtttcgtaactataacggtcctaaggtagcgaacatcttgttctggggtttcacactatctttagagaaagtgttaagttaattaagttatcttaattaagagcataattatactgatttgtctctcgttgatagagtctatcattctgttactaaaaatttgacaactcggtttgctgacctactggttactgtatcacttacccgagttacgagaatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagacgatgggctgggaggcctccaccgagcgcctgtacccccgcgacggcgtgctgaagggcgagacacacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtaggcttctcgagcccgggtagggataacagggtaatgtcgaccccgaagacattaaactacggttctttaagtagatccgtgtctgaagttttaggttcaatttaaacctacgagattgacattctcgactgatcttgattgatcggtaagtcttttgtaatttaattttctttttgattttattttaaattgttatctgtttctgtgtatagactgtttgagatcggcgtttggccgactcattgtcttaccataggggaacggactttgtttgtgttgttattttatttgtattttattaaaattctcaacgatctgaaaaagcctcgcggctaagagattgttggggggtgagtaagtacttttaaagtgatgatggttacaaaggcaaaaggggtaaaacccctcgcctacgtaagcgttattacgcccgtctgtacttatatcagtacactgacgagtccctaaaggacgaaacgggaggaattctgcaggagtggggaggcacgatggccgctttggtcgatcgacgggatcgatcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggatcgatccactagctgtcaggagacgggacaaggatgcgagctagcctgcaggaattgttgattttgtgatgactgatggcaggatatatgcggttgtaattcatttttattgtctaaatttctgtatttgtttgtttgttcggttgtaaatttttttggaagaacaagaaaagaaaaaacacccgttagggtgtttttagttagtgtggcgcgccgacttgcgacatgcggtcctttgcaatcaactattagaaaaattcatccagcatcagatgaaattgcagtttgttcatatccggattatcaatgccatatttctgaaacagacgtttttgcaggctcgggctaaattcgcccaggcagttccacagaatggccagatcctgataacgatccgcaatgcccacacggcccacatcaatgcagccaatcagtttgccttcatcgaaaatcaggttatccaggctaaaatcgccgtgggtcaccacgctatccgggctaaacggcagcagtttatgcatttctttccacacctgttccaccggccagccgttacgttcatcatcaaaatcgctcgcatccaccaggccgttgttcatacggctctgcgcctgggccagacgaaacacacgatcgctgttaaacgggcagttgcacaccggaatgctatgcagacgacgcagaaacacggccagcgcatccacaatgttttcgccgctatccggatattcttccagcacctgaaacgcggttttgcccggaatcgcggtggtcagcagccacgcatcatccggggtgcgaataaaatgtttaatggtcggcagcggcataaattcggtcagccagttcagacgcaccatttcatcggtcacatcgttcgccacgctgcctttgccatgtttcagaaacagttccggcgcatccggtttgccatacagacgataaatggtcgcgccgctctgacccacgttatcacgcgcccatttatagccatacagatccgcatccatgttgctgttcagacgcggacggctacagctcgtttcacgctgaatatggctcataacaccccttgtattactgtttatgtaagcagacagttttattgttcatgatgatatatttttatcttgtgcaatgtaacatcagagattttgagacacaagatcggattggcggttatgcggttctaccggcgcggcagcgttacccgtgtcggcggctccaacggctcgccatcgtccagaaaacacggctcatcgggcatcggcaggcgctgctgcccgcgccgttcccattcctccgtttcggtcaaggctggcaggtctggttccatgcccggaatgccgggctggctgggcggctcctcgccggggccggtcggtagttgctgctcgcccggatacagggtcgggatgcggcgcaggtcgccatgccccaacagcgattcgtcctggtcgtcgtgatcaaccaccacggcggcactgaacaccgacaggcgcaactggtcgcggggctggccccacgccacgcggtcattgaccacgtaggccgacacggtgccggggccgttgagcttcacgacggagatccagcgctcggccaccaagtccttgactgcgtattggaccgtccgcaaagaacgtccgatgagcttggaaagtgtcttctggctgaccaccacggcgttctggtggcccatctgcgccacgaggtgatgcagcagcattgccgccgtgggtttcctcgcaataagcccggcccacgcctcatgcgctttgcgttccgtttgcacccagtgaccgggcttgttcttggcttgaatgccgatttctctggactgcgtggccatgcttatctccatgcggtaggggtgccgcacggttgcggcaccatgcgcaatcagctgcaacttttcggcagcgcgacaacaattatgcgttgcgtaaaagtggcagtcaattacagattttctttaacctacgcaatgagctattgcggggggtgccgcaatgagctgttgcgtaccccccttttttaagttgttgatttttaagtctttcgcatttcgccctatatctagttctttggtgcccaaagaagggcacccctgcggggttcccccacgccttcggcgcggctccccctccggcaaaaagtggcccctccggggcttgttgatcgactgcgcggccttcggccttgcccaaggtggcgctgcccccttggaacccccgcactcgccgccgtgaggctcggggggcaggcgggcgggcttcgcccttcgactgcccccactcgcataggcttgggtcgttccaggcgcgtcaaggccaagccgctgcgcggtcgctgcgcgagccttgacccgccttccacttggtgtccaaccggcaagcgaagcgcgcaggccgcaggccggaggcttttccccagagaaaattaaaaaaattgatggggcaaggccgcaggccgcgcagttggagccggtgggtatgtggtcgaaggctgggtagccggtgggcaatccctgtggtcaagctcgtgggcaggcgcagcctgtccatcagcttgtccagcagggttgtccacgggccgagcgaagcgagccagccggtggccgctcgcggccatcgtccacatatccacgggctggcaagggagcgcagcgaccgcgcagggcgaagcccggagagcaagcccgtaggggg

SEQ ID NO: 32 - DNA, 18011 bp, synthetic construct
  misc_feature 1..18011: note = DNA Construct
  source 1..18011: mol_type = other DNA
attacaacggtatatatcctgccagtcagcatcatcacaccaaaagttaggcccgaatagtttgaaattagaaagctcgcaattgaggtctccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattcgagctcggtacccggggatcccccctcagaagaccagagggctattgagacttttcaacaaagggtaatatcgggaaacctcctcggattccattgcccagctatctgtcacttcatcgaaaggacagtagaaaaggaaggtggctcctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggatccggcgcgccataaaacattgcacctatggtgttgccctggctggggtatgtcagtgatcgcagtagaatgtactaattgacaagttggagaatacggtagaacgtccttatccaacacagcctttatccctctccctgacgaggtttttgtcagtgtaatatttctttttgaactatccagcttagtaccgtacgggaaagtgactggtgtgcttatctttgaaatgttactttgggtttcggttctttaggttagtaagaaagcacttgtcttctcatacaaaggaaaacctgagacgtatcgcttacgaaagtagcaatgaaagaaaggtggtggttttaatcgctaccgcaaaaacgatggggtcgttttaattaacttctcctacgcaagcgtctaaacggacgttggggttttgctagtttctttagagaaaactagctaagtctttaatgttatcattagagatggcataaatataatacttgtgtctgctgataagatcattttaatttggacgattagacttgttgaactacaggttactgaatcacttgcgctaatcaacaagcttcctttgcttcatccattagatctagttttagagctagaaatagcaagttaaaataaggctagtccgttatcaacttgaaaaagtggcaccgagtcggtgctttttttcctaagggatttaaggacgtgaactctgttgagatctctgtgaaattcagagggtgggtgataccatattcactgatgccattagcgacatctaaatagggctaattgtgactaatttgagggaatttcctttaccattgacgtcagtgtcgttggtagcatttgagtttcgtaactataacggtcctaaggtagcgaacatcttgttctggggtttcacactatctttagagaaagtgttaagttaattaagttatcttaattaagagcataattatactgatttgtctctcgttgatagagtctatcattctgttactaaaaatttgacaactcggtttgctgacctactggttactgtatcacttacccgagttacgagaatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagacgatgggctgggaggcctccaccgagcgcctgtacccccgcgacggcgtgctgaagggcgagacacacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtaggcttctcgagcccgggtagggataacagggtaatgtcgaccccgaagacattaaactacggttctttaagtagatccgtgtctgaagttttaggttcaatttaaacctacgagattgacattctcgactgatcttgattgatcggtaagtcttttgtaatttaattttctttttgattttattttaaattgttatctgtttctgtgtatagactgtttgagatcggcgtttggccgactcattgtcttaccataggggaacggactttgtttgtgttgttattttatttgtattttattaaaattctcaacgatctgaaaaagcctcgcggctaagagattgttggggggtgagtaagtacttttaaagtgatgatggttacaaaggcaaaaggggtaaaacccctcgcctacgtaagcgttattacgcccgtctgtacttatatcagtacactgacgagtccctaaaggacgaaacgggaggaattctgcaggagtggggaggcacgatggccgctttggtcgatcgacgggatcgatcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggatcgatccactagttctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgcaagctcctcgaggagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatccgtcctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctctattttctccataataatgtgtgagtagttcccagataagggaattagggttcttatagggtttcgctcagatccgtcgacgtcgaggaattccccggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactscaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccggaattcccgggggaggaaattacactgaggaaggagaagatgacgacgatgagatggacgatgaaggggaggctggtggagcggaaccaagagagtgtcagatcggaaaccttatcaattatccgatcattgctttagggtcatgcgatctttccgcataattcccgtcgccgacacctaataaagtcggctaatctatgtgattgagtgtgtcttgactttgttattttgcatgtttccaatgtcatttagtaacgaaataaacgttatcctcttctaaaagcaggctgtgttttcggcaaacatcgccacccatcgctagtttttctaaaagtgttctaagctagcctggtaataatctatacgagcttatatttctaatcattgccgaaaaatcctgtttcgaaataattttgtaactctctttaatatcaccacgatcacacaagaagaagaattaaatataacatttatcagcccacgatgaacatggcgaaaattacaagcgaacacaattgtcttattcattaataattaattcaacaaagttctcagacactccttgatttaacagaagattttataagcacctctatgatgcaacaaccaaaacgacatagacttcgaaatcccgccccataaaaactagatagcaccatctaactccacgtcttcaagagcagcctccgggaggaaagctgaacagcccacagcgatcaaaagataatccaatggttggccggcgcttggcattttccttttccaaacgtccttgctttgaccaaaagtgttccggacgactcctatgcgttcgaactcgttgctggcctgaattgctagaaagtacgaggatgaatattcatttttgctgtagttgatccgaccaccgtctccgcttaaggcttgcagttgcgccctcacctgccggaatgtataaccatcagaatagggttccagtgtcgtggcaagcaatccatctgaagcatttgaaactagaaccattctttgccgacataaccgataattctctttatcgagataaatgtacagaaattccccgccaggattgatgatgtcgccagacttagtgtgtagatcggtttgaaacttttcccgataaacttttgaaagctccgagagttcaccccctcgaggcgatcgtgaagtttctcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcccggacatgaagccatttacaattgaatatatcctgccgccgctgccgctttgcacccggtggagcttgcatgttggtttctacgcagaactgagccggttaggcagataatttccattgagaactgagccatgtgcaccttccccccaacacggtgagcgacggggcaacggagtgatccacatgggacttttaaacatcatccgtcggatggcgttgcgagagaagcagtcgatccgtgagatcagccgacgcaccgggcaggcgcgcaacacgatcgcaaagtatttgaacgcaggtacaatcgagccgacgttcacagatccagatcctacaaggtagaatccgcctgagtcgcaagggtgacttcgcctatattggacgacggcgcgcagagggcgacctctttttgggttacgattgtaggattatcactaaaacaatacatgaacatattcaaatggcaatctctctaaggcattggaaataaatacaaataacagttgggtggagtttttcgacctgagggcgttaaccttctgttaacctaaaagctcttgcccaaacagcagaatcggcgctaattgccagcggcggaacttttccagtttcgcgaaaaatatcgccactggcaaggaatgggtttgagatggcgaagtctgtcctaaaagcagcgcctgtagttgtagggttgacggccttgatggagcgtcatgccgatgccctctcgagccaacttcaagcacatcatcttaaggttttcccgccgcattccgagaagggcattcgaacattcgggccatcggaggcgtccaagctgctcggcgttggcgagtcatatttacggcagaccgcgtctgagatgccagagttgaatgttagcatgagcccgggtggcaggcgaatgttctcaattgaagatatccatgtgattcggaagtatatggatcaggtcggccgcgggaaccggcgctacctgccacatcgtcgaggcggcgagcagcttcaggttatctctgtgatgaatttcaaaggtgggtcgggtaagaccaccaccgccgcgcatctggcgcagtacctcgctatgcgcggatatcgagtcttggccattgatctcgatcctcaagcgagcctttctgcactctttgggagccaaccggagacggacgttggccgaacgaaacgctctacggcgctataggtatgatgatgagcaggtggcaatcgaacgagtcgtccgagggacttacattcccgacctccacctgattcctggtaaccttgagctgatggagtttgaacacgatacgccacgcgcgctgatgaaccgcaaagagggcgacacgctcttttatggtcgcatcagccaagtaattgaagatatcgcggataactatgacgtcgtggtcatcgactgccctccccagcttgggtatctcacgctatccgcattgactgcggcgacgtccattcttgtcacggtccatccgcagatgctggatgtgatgtcgatgaaccagtttctggcaatgacatcgaaccttttgcgtgaaatcgagaatgctggcgccaagttcaagtttaattggatgcgctatctgataacccgtttcgaaccgagcgacggaccacagaaccaaatggtaggttatctgcggtcgatttttggcgaaaatgtcctcaattttccgatgcttaaaaccaccgcggtttcggacgctggcctgacaaaccagactctattcgaagtggagcgtggcctgttcacgcgctcgacctatgatcgagccttggaggcgatgaacgccgtcaacgacgagatcgaaacactgatcaaaaaagcatggggtaggcccacatgagccggaagcacatccttggcgtctcaactgacgcccctgagacgtcgcccgccgacaataggacggcaaagaaccgctccatgccgctcctcggcgtaacaaggaaggagcgcgatccggcaacgaagctcacagcgaacattggtaacgcactgcgagagcaaaacgatcgtcttagccgtgccgaagagatcgagcggcgtctcgctgaagtcaggcagtgatagagttggatgcctcgtcaatagaaccgtctttcgtgcaggatcgtatgcgaggggacattgacggctccttacttcgatccgggaacaaggacagcaagtccaatccttgtgcgaccgcatccgagccagccgggccgatatcaggttgccttcggccaccgccggctacgcgccgtttcagaactcggacttccggtcagagcggtcgttcgcgaactgacggacgagcaagtggtcgtagcacagggtcaggaaaacaatgagcgcgaagatcttaccttcatcgaaaaggcgcgcttcgcacatcgcctgaacaggcagttttctcgagagattgtcatcgccgcgatgtcgatcgacaagagcaatttgtccaagatgcttctgctcgtcgacgccctcccctctgaactgaccgatgctattggtgccgctcctggtgttggacggccgagttggcaacaacttgccgagctgattgagaaagtttcttcaccggccgacgtggctaaatatgctatgtcggaggaagttcaagcgctgccatcggcagaacgattcaaggcggtgatcgctagtctgaagcccagtcgggttgcgcgtggacttcccgaggtcatggccaccccagacggcaccagaattgcacaggtgacgcagagcaaggccaaactggaaatcacgattgacaggaaggcgacgcccgattttgcgaccttcgtgctcgatcatgtgccagcgctgtatcaagcgtaccacgctgagaaccaacggaaacggggagagtaaaccgcaaaagaaaagagccccctcaacgtcgccgtcgcggaagcccttctgtctctctagcgcgaacagaatcgcatttcctcgaatcctcgtcaagagtttttagcgccgttttggtgagctgatttcctttgcctgctgaaaggtgaaagatgatgcagacaggaagtgtaacgacgccattcgggcggcggccaatgacgcttgcgcttgtgcggcgccagacggcgctggccgatatcaaacaaggcaagacagcggacaagtggaaggtctttagagacgcgtccgcggccatggaactacttggaatccagtccaacagtcttgccgtccttgatgcgctattgagctttcacccggaaacggagttgcgtcaggaggcacagctgatcgtcttcccgtcgaatgctcagcttgcccttcgggcgcatgggatggctggcgcgactttgcgtaggcacatcgccatgctcgtggagtcaggcttgatcgtccggaaggatagcgccaacggaaagcgttacgctcgtaaggatggcgctggtcagatcgagcgcgcgtttggcttcgatttgtctccgcttctcgcgcggtccgaagagctagcgatgatggcacagcaggtgatggccgatcgagcagcattcaggatggccaaagaaagtctgacgatttgccgacgggacgttcggaagctaattacggcagctatggaagagggagcggagggcgactggcaagctgtcgaggaagtctatgtggaacttgtgggtagaattccacgcgccccgacgcttgctgatgtagagtcaattctcgaagagatgtggatgctccaggaagagataatcaaccggttggaaattagagacaattcagaaaataatagcaccaatgctgcccagagcgagcagcacatacagaattcaaaacccgaatccgttaatgaacttgaacctcgctctgaaaaggagcagggcgctaagccgagtgaaatagaccgggcaaggagcgagccgataaaagcgttccccctcgggatgatcctgaaagcatgcccgaccattggcaattatgggccgagcggtgcggttgctagctggcgtgacctcatgtcggctgcggtggtggttcggtctatgctgggggtcagcccgtcggcttaccaagacgcgtgtgaggcaatgggaccggagaatgcggcagcagcgatggcgtgcattttggagcgagcgaacttcatcaattcgcccgggggctatctccgagatctgacacggcggagcgagctcgggaagttttcacttggcccgatgataatggcgctcttgaaggctagcgggcaggggacgttgcggtttggctagaattagcgagtatggagcaggatggtctgtggtcagctgaccacagacctaataggttgaaaacatgagcgttttttggatgatcgacagaccatccgattcccggagtaccaagcgtgctctgatgggagcgataacattactcaacaagcacgaaggccccatgccgatcgttgatcgtgaaggagagcctgctctacatgcggcggtattttgccggccgaggcatgtagtcgcggagcactgcctatttactgccctaggcacaaacgttgactcttggatcgagctggcagacaaagcaataacccacacagaggacgattaatggctgacgaagagatccagaatccgccggacggtactgctgctgccgaagttgagccggctgctcctagaggtagaagagcaaagaaagcaccagccgaaacagcccgcacgggatcgttcaaatccgtgaagccgaaaacccgcggcctcagcaaccgagaaaaactggagaagatcggtcaaatcgaagctcaggtcgctggcggcgcaaccttgaaggacgccgttaagatcgtgggtatttccgttcagacctattatcaatggaagagagctgcggttcaacctgtctcacagaatccggccgtgtctgtttcagttgacgatgaactcggcgagttcatccaactcgaggaggaaaatcggcggctcagaaagctagcttctctctgaaaaattgcgcgcagaaaacgcggacttacgcaaaagacttggaatcaactagcggcctttgggggtccctcgctaaatcaacttgacgcgcatctctcaatgaatgaatgccttggaaaatttccgcgcattctgatcgttgcgctatcaagcaatcccaatcttctggcatatgaaaaaaccacgacccaaattcgtcgtcgaatataagacgaacaggcgacaagccaaggcccgaccaacgtcaatctggggcaatctgggtttgcaggcggtgggccgtgcggtcgcggccgacggagtcgtgccgaagagtagtctgccgcaagtccgttccgtcctggagaaggttgtcgcgtccaaggttaccgtcgtggtccctcggtgctgccaggttttgaggaagagcttgccgctatcgaggcggaaaaccgtcgtctgaagcgcctcttgatcgtgaagctccgtgaggagaacgacaggcttaagttcgcgcttcgacgatttgggggcgcctgagcacgatgctggcccacttgcttcagcgggcaaacaacatggagtggagatctaatgatcgcggcgcttgaacatcgctaggggtcttcggcgccgctgaaccgtgctccattttgcactcggagacacagcctcccaaaaacctggcgccgctcgttggtgaactcctcaacatcgagcggatcaagggaaactggaatgacatccttcgacccgtcacggcgatccgctctggccaggtccaacctacggccatgcccgcaaagccaacggcatttccacgtcaagcacattgacccactcggctgacggtacatcagctggcgagctcatccggacccagacgaactgcgttgatctcaggccgttgcggcagaaaacctccatcctcgcagccgatccaacactttcctgtggtaatgcctatttcagatgaaacagccttttttaaacttggcgcgtcattctgatattatggccacacctctacgatctagggagtgcgagaagtggaaagaaatgaagaacccgatttgcacacacatacctcgatgcggaataaaaatgcatcatcgcttggtgaggccctggatgacgatgcgatcgccaacatcgcgcatcacattgttacagacgacccggtggaaacgtcaaaaaacttcacaaaacttaagctgattaacaaatcagtgaaacagggtttcgaacgcagtgcgtacggaaaattccatcgacgcattagccggcttggtattgctagtaaggcgctatatgacaacgctgtgccgaaggaaggattcgtagaacatcctgaatatgctccagctctgcaaagggattatgcgttggcaggagagcgaattgaggcggttggtggaactttcaggttgcaaacacctcagcggaaatcggctcttgtagatcatatcttgagtatgcccgaaggtttcgatcaagctcatgcccttgagtctatagcccctcatgttggcgatctaagcgcggaagatcgccatcgtgttgtgaacaaaatgctcgagcactttgccaccccgtttcaagaaggggggctgccaaccgacgaaaatttttgtggaacgtacgctttgtccaaggcgtttcctcatatggaagacagcctaaaagcaaagatgctggatgtggtgttagagcatcctcacttagccaacctccattggacgcagagagaactagttgaagggtggcatcaagaccgtgaagctgagaagtcagcctggcgttccaatcaaaagccaattgtcgaggggcgacttgcagacctcgaacgatcgatccacagtgatatcgaggtgacaaacaggagtgattttgcgaggatgcgttcagccaggcccttgcaacaagctataagcgatacgttcgatcttgcgcgtgaggagctcaaaagccgccctcgtgaagggcggggtcgataattacatctattggtctttctgccgaacctctatgaaaatccgcacgtcaaatgtggcttttacttcccgatttcaattgaagcgctaagctgaaaatccgtccccatttctgacctaacggtttcatcaccggggcagggagcggcgcttaaaacggtgacggccaccaccaaaggacatgttcgctcacgcggtgcctccgtgcttctttgcaagagcctgcttcaggctgatgatctccttcagtgcgttggtgtgaatccgctccaggcgtttcagttcgctctccaaataggcgttgcgcttatccagcctcgtccgatcttcttccaagctctccattttcactagcgcgttatggtaattcaaccgtaactcctcaaaaaaagcacgttcggcgacgaaatgggtttcggaatcgagttcaggtatgtcgccgacatgcacgacgcgaaccgccttgccggcagcggcgagccgggcgcggtaagcgcgttgaatttccgcttgggtcagcgcgtttggcttgcggggccgcgaggaaatttgggagtaaccataacggttatttatcataacgcgttatcactggatagcatcggcgcaggcggatggcgcgcttcgccgggtcgaaatcaatatttccagatcgagatggattgaagacctgaagcccgttccatacagaagctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaacctgcgctcgttcgccaggccaggacagaaatgcctcgacttcgctgctgcccaaggttgccggggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaagtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcggccgcaaagcgctccggcttaactcaggcgttagtcccatcacccgctttatgcgcgcagccacttatttcagcgctccgccctgttccaaaggctttctagccttttgggcctccagcgcttgtgcagcaagcgccaccagctatccttcttgcagcgctgcgccgctttcgtggtccagcggtttttcttgggctgcgcccttcttcaaggccgggcgctcccttttggccttcgggtctaactctgcggtcaagcggacccgcattctgcgggccgcttaccttggccgttagacatcatgagggtagcggtgaccatcgaaatttcgaaccaactatcagaggtgctaagcgtcattgagcgccatctggaatcaacgttgctggccgtgcatttgtacggctccgcagtggatggcggcctgaagccatacagcgatattgatttgttggttactgtggccgtaaagctctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcagggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatcctttctcagagcccagacgccttggttaggttggtgcggaaccgatcccgccgccgctggtatttgcgcgtcatctcggccgaggcgtggcctagttgtttctgaacgtaacgctcctcaatgtcggccgaggaggcaagaccggcgcgcagcgagtgccctgagaacagcagcgcccgttcccttctggaaagatcggaacggacgccggcggcgagtgcggtctgcttgaccaggcgggcgacatgcttgtcggagagccgctcgacgtccacggtcttgttgtccttgaagatgcgccggaacaggggaccgcgcgcgatcctgccgaagcggatctgatcaccggcggttccgaagctcggtcccgtgggtgttctgtcgtctcgttgtacaacgaaatccattcccattccgcgctcaagatggcttcccctcggcagttcatcagggctaaatcaatctagccgacttgtccggtgaaatgggctgcactccaacagaaacaatcaaacaaacatacacagcgacttattcacacgagctcaa

SEQ ID NO: 33 - DNA, 18032 bp, synthetic construct
  misc_feature 1..18032: note = DNA Construct
  source 1..18032: mol_type = other DNA
attacaacggtatatatcctgccagtcagcatcatcacaccaaaagttaggcccgaatagtttgaaattagaaagctcgcaattgaggtctccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattcgagctcggtacccggggatcccccctcagaagaccagagggctattgagacttttcaacaaagggtaatatcgggaaacctcctcggattccattgcccagctatctgtcacttcatcgaaaggacagtagaaaaggaaggtggctcctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggatccggcgcgccataaaacattgcacctatggtgttgccctggctggggtatgtcagtgatcgcagtagaatgtactaattgacaagttggagaatacggtagaacgtccttatccaacacagcctttatccctctccctgacgaggtttttgtcagtgtaatatttctttttgaactatccagcttagtaccgtacgggaaagtgactggtgtgcttatctttgaaatgttactttgggtttcggttctttaggttagtaagaaagcacttgtcttctcatacaaaggaaaacctgagacgtatcgcttacgaaagtagcaatgaaagaaaggtggtggttttaatcgctaccgcaaaaacgatggggtcgttttaattaacttctcctacgcaagcgtctaaacggacgttggggttttgctagtttctttagagaaaactagctaagtctttaatgttatcattagagatggcataaatataatacttgtgtctgctgataagatcattttaatttggacgattagacttgttgaactacaggttactgaatcacttgcgctaatcaacaagcttccttccctaggaggaagatccagtgttttagagctagaaatagcaagttaaaataaggctagtccgttatcaacttgaaaaagtggcaccgagtcggtgctttttttcctaagggatttaaggacgtgaactctgttgagatctcatgtgagcaaaaggcgatctctgtgaaattcagagggtgggtgataccatattcactgatgccattagcgacatctaaatagggctaattgtgactaatttgagggaatttcctttaccattgacgtcagtgtcgttggtagcatttgagtttcgtaactataacggtcctaaggtagcgaacatcttgttctggggtttcacactatctttagagaaagtgttaagttaattaagttatcttaattaagagcataattatactgatttgtctctcgttgatagagtctatcattctgttactaaaaatttgacaactcggtttgctgacctactggttactgtatcacttacccgagttacgagaatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagacgatgggctgggaggcctccaccgagcgcctgtacccccgcgacggcgtgctgaagggcgagacacacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtaggcttctcgagcccgggtagggataacagggtaatgtcgaccccgaagacattaaactacggttctttaagtagatccgtgtctgaagttttaggttcaatttaaacctacgagattgacattctcgactgatcttgattgatcggtaagtcttttgtaatttaattttctttttgattttattttaaattgttatctgtttctgtgtatagactgtttgagatcggcgtttggccgactcattgtcttaccataggggaacggactttgtttgtgttgttattttatttgtattttattaaaattctcaacgatctgaaaaagcctcgcggctaagagattgttggggggtgagtaagtacttttaaagtgatgatggttacaaaggcaaaaggggtaaaacccctcgcctacgtaagcgttattacgcccgtctgtacttatatcagtacactgacgagtccctaaaggacgaaacgggaggaattctgcaggagtggggaggcacgatggccgctttggtcgatcgacgggatcgatcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggatcgatccactagttctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgcaagctcctcgaggagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatccgtcctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctctattttctccataataatgtgtgagtagttcccagataagggaattagggttcttatagggtttcgctcagatccgtcgacgtcgaggaattccccggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactscaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccggaattcccgggggaggaaattacactgaggaaggagaagatgacgacgatgagatggacgatgaaggggaggctggtggagcggaaccaagagagtgtcagatcggaaaccttatcaattatccgatcattgctttagggtcatgcgatctttccgcataattcccgtcgccgacacctaataaagtcggctaatctatgtgattgagtgtgtcttgactttgttattttgcatgtttccaatgtcatttagtaacgaaataaacgttatcctcttctaaaagcaggctgtgttttcggcaaacatcgccacccatcgctagtttttctaaaagtgttctaagctagcctggtaataatctatacgagcttatatttctaatcattgccgaaaaatcctgtttcgaaataattttgtaactctctttaatatcaccacgatcacacaagaagaagaattaaatataacatttatcagcccacgatgaacatggcgaaaattacaagcgaacacaattgtcttattcattaataattaattcaacaaagttctcagacactccttgatttaacagaagattttataagcacctctatgatgcaacaaccaaaacgacatagacttcgaaatcccgccccataaaaactagatagcaccatctaactccacgtcttcaagagcagcctccgggaggaaagctgaacagcccacagcgatcaaaagataatccaatggttggccggcgcttggcattttccttttccaaacgtccttgctttgaccaaaagtgttccggacgactcctatgcgttcgaactcgttgctggcctgaattgctagaaagtacgaggatgaatattcatttttgctgtagttgatccgaccaccgtctccgcttaaggcttgcagttgcgccctcacctgccggaatgtataaccatcagaatagggttccagtgtcgtggcaagcaatccatctgaagcatttgaaactagaaccattctttgccgacataaccgataattctctttatcgagataaatgtacagaaattccccgccaggattgatgatgtcgccagacttagtgtgtagatcggtttgaaacttttcccgataaacttttgaaagctccgagagttcaccccctcgaggcgatcgtgaagtttctcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcccggacatgaagccatttacaattgaatatatcctgccgccgctgccgctttgcacccggtggagcttgcatgttggtttctacgcagaactgagccggttaggcagataatttccattgagaactgagccatgtgcaccttccccccaacacggtgagcgacggggcaacggagtgatccacatgggacttttaaacatcatccgtcggatggcgttgcgagagaagcagtcgatccgtgagatcagccgacgcaccgggcaggcgcgcaacacgatcgcaaagtatttgaacgcaggtacaatcgagccgacgttcacagatccagatcctacaaggtagaatccgcctgagtcgcaagggtgacttcgcctatattggacgacggcgcgcagagggcgacctctttttgggttacgattgtaggattatcactaaaacaatacatgaacatattcaaatggcaatctctctaaggcattggaaataaatacaaataacagttgggtggagtttttcgacctgagggcgttaaccttctgttaacctaaaagctcttgcccaaacagcagaatcggcgctaattgccagcggcggaacttttccagtttcgcgaaaaatatcgccactggcaaggaatgggtttgagatggcgaagtctgtcctaaaagcagcgcctgtagttgtagggttgacggccttgatggagcgtcatgccgatgccctctcgagccaacttcaagcacatcatcttaaggttttcccgccgcattccgagaagggcattcgaacattcgggccatcggaggcgtccaagctgctcggcgttggcgagtcatatttacggcagaccgcgtctgagatgccagagttgaatgttagcatgagcccgggtggcaggcgaatgttctcaattgaagatatccatgtgattcggaagtatatggatcaggtcggccgcgggaaccggcgctacctgccacatcgtcgaggcggcgagcagcttcaggttatctctgtgatgaatttcaaaggtgggtcgggtaagaccaccaccgccgcgcatctggcgcagtacctcgctatgcgcggatatcgagtcttggccattgatctcgatcctcaagcgagcctttctgcactctttgggagccaaccggagacggacgttggccgaacgaaacgctctacggcgctataggtatgatgatgagcaggtggcaatcgaacgagtcgtccgagggacttacattcccgacctccacctgattcctggtaaccttgagctgatggagtttgaacacgatacgccacgcgcgctgatgaaccgcaaagagggcgacacgctcttttatggtcgcatcagccaagtaattgaagatatcgcggataactatgacgtcgtggtcatcgactgccctccccagcttgggtatctcacgctatccgcattgactgcggcgacgtccattcttgtcacggtccatccgcagatgctggatgtgatgtcgatgaaccagtttctggcaatgacatcgaaccttttgcgtgaaatcgagaatgctggcgccaagttcaagtttaattggatgcgctatctgataacccgtttcgaaccgagcgacggaccacagaaccaaatggtaggttatctgcggtcgatttttggcgaaaatgtcctcaattttccgatgcttaaaaccaccgcggtttcggacgctggcctgacaaaccagactctattcgaagtggagcgtggcctgttcacgcgctcgacctatgatcgagccttggaggcgatgaacgccgtcaacgacgagatcgaaacactgatcaaaaaagcatggggtaggcccacatgagccggaagcacatccttggcgtctcaactgacgcccctgagacgtcgcccgccgacaataggacggcaaagaaccgctccatgccgctcctcggcgtaacaaggaaggagcgcgatccggcaacgaagctcacagcgaacattggtaacgcactgcgagagcaaaacgatcgtcttagccgtgccgaagagatcgagcggcgtctcgctgaagtcaggcagtgatagagttggatgcctcgtcaatagaaccgtctttcgtgcaggatcgtatgcgaggggacattgacggctccttacttcgatccgggaacaaggacagcaagtccaatccttgtgcgaccgcatccgagccagccgggccgatatcaggttgccttcggccaccgccggctacgcgccgtttcagaactcggacttccggtcagagcggtcgttcgcgaactgacggacgagcaagtggtcgtagcacagggtcaggaaaacaatgagcgcgaagatcttaccttcatcgaaaaggcgcgcttcgcacatcgcctgaacaggcagttttctcgagagattgtcatcgccgcgatgtcgatcgacaagagcaatttgtccaagatgcttctgctcgtcgacgccctcccctctgaactgaccgatgctattggtgccgctcctggtgttggacggccgagttggcaacaacttgccgagctgattgagaaagtttcttcaccggccgacgtggctaaatatgctatgtcggaggaagttcaagcgctgccatcggcagaacgattcaaggcggtgatcgctagtctgaagcccagtcgggttgcgcgtggacttcccgaggtcatggccaccccagacggcaccagaattgcacaggtgacgcagagcaaggccaaactggaaatcacgattgacaggaaggcgacgcccgattttgcgaccttcgtgctcgatcatgtgccagcgctgtatcaagcgtaccacgctgagaaccaacggaaacggggagagtaaaccgcaaaagaaaagagccccctcaacgtcgccgtcgcggaagcccttctgtctctctagcgcgaacagaatcgcatttcctcgaatcctcgtcaagagtttttagcgccgttttggtgagctgatttcctttgcctgctgaaaggtgaaagatgatgcagacaggaagtgtaacgacgccattcgggcggcggccaatgacgcttgcgcttgtgcggcgccagacggcgctggccgatatcaaacaaggcaagacagcggacaagtggaaggtctttagagacgcgtccgcggccatggaactacttggaatccagtccaacagtcttgccgtccttgatgcgctattgagctttcacccggaaacggagttgcgtcaggaggcacagctgatcgtcttcccgtcgaatgctcagcttgcccttcgggcgcatgggatggctggcgcgactttgcgtaggcacatcgccatgctcgtggagtcaggcttgatcgtccggaaggatagcgccaacggaaagcgttacgctcgtaaggatggcgctggtcagatcgagcgcgcgtttggcttcgatttgtctccgcttctcgcgcggtccgaagagctagcgatgatggcacagcaggtgatggccgatcgagcagcattcaggatggccaaagaaagtctgacgatttgccgacgggacgttcggaagctaattacggcagctatggaagagggagcggagggcgactggcaagctgtcgaggaagtctatgtggaacttgtgggtagaattccacgcgccccgacgcttgctgatgtagagtcaattctcgaagagatgtggatgctccaggaagagataatcaaccggttggaaattagagacaattcagaaaataatagcaccaatgctgcccagagcgagcagcacatacagaattcaaaacccgaatccgttaatgaacttgaacctcgctctgaaaaggagcagggcgctaagccgagtgaaatagaccgggcaaggagcgagccgataaaagcgttccccctcgggatgatcctgaaagcatgcccgaccattggcaattatgggccgagcggtgcggttgctagctggcgtgacctcatgtcggctgcggtggtggttcggtctatgctgggggtcagcccgtcggcttaccaagacgcgtgtgaggcaatgggaccggagaatgcggcagcagcgatggcgtgcattttggagcgagcgaacttcatcaattcgcccgggggctatctccgagatctgacacggcggagcgagctcgggaagttttcacttggcccgatgataatggcgctcttgaaggctagcgggcaggggacgttgcggtttggctagaattagcgagtatggagcaggatggtctgtggtcagctgaccacagacctaataggttgaaaacatgagcgttttttggatgatcgacagaccatccgattcccggagtaccaagcgtgctctgatgggagcgataacattactcaacaagcacgaaggccccatgccgatcgttgatcgtgaaggagagcctgctctacatgcggcggtattttgccggccgaggcatgtagtcgcggagcactgcctatttactgccctaggcacaaacgttgactcttggatcgagctggcagacaaagcaataacccacacagaggacgattaatggctgacgaagagatccagaatccgccggacggtactgctgctgccgaagttgagccggctgctcctagaggtagaagagcaaagaaagcaccagccgaaacagcccgcacgggatcgttcaaatccgtgaagccgaaaacccgcggcctcagcaaccgagaaaaactggagaagatcggtcaaatcgaagctcaggtcgctggcggcgcaaccttgaaggacgccgttaagatcgtgggtatttccgttcagacctattatcaatggaagagagctgcggttcaacctgtctcacagaatccggccgtgtctgtttcagttgacgatgaactcggcgagttcatccaactcgaggaggaaaatcggcggctcagaaagctagcttctctctgaaaaattgcgcgcagaaaacgcggacttacgcaaaagacttggaatcaactagcggcctttgggggtccctcgctaaatcaacttgacgcgcatctctcaatgaatgaatgccttggaaaatttccgcgcattctgatcgttgcgctatcaagcaatcccaatcttctggcatatgaaaaaaccacgacccaaattcgtcgtcgaatataagacgaacaggcgacaagccaaggcccgaccaacgtcaatctggggcaatctgggtttgcaggcggtgggccgtgcggtcgcggccgacggagtcgtgccgaagagtagtctgccgcaagtccgttccgtcctggagaaggttgtcgcgtccaaggttaccgtcgtggtccctcggtgctgccaggttttgaggaagagcttgccgctatcgaggcggaaaaccgtcgtctgaagcgcctcttgatcgtgaagctccgtgaggagaacgacaggcttaagttcgcgcttcgacgatttgggggcgcctgagcacgatgctggcccacttgcttcagcgggcaaacaacatggagtggagatctaatgatcgcggcgcttgaacatcgctaggggtcttcggcgccgctgaaccgtgctccattttgcactcggagacacagcctcccaaaaacctggcgccgctcgttggtgaactcctcaacatcgagcggatcaagggaaactggaatgacatccttcgacccgtcacggcgatccgctctggccaggtccaacctacggccatgcccgcaaagccaacggcatttccacgtcaagcacattgacccactcggctgacggtacatcagctggcgagctcatccggacccagacgaactgcgttgatctcaggccgttgcggcagaaaacctccatcctcgcagccgatccaacactttcctgtggtaatgcctatttcagatgaaacagccttttttaaacttggcgcgtcattctgatattatggccacacctctacgatctagggagtgcgagaagtggaaagaaatgaagaacccgatttgcacacacatacctcgatgcggaataaaaatgcatcatcgcttggtgaggccctggatgacgatgcgatcgccaacatcgcgcatcacattgttacagacgacccggtggaaacgtcaaaaaacttcacaaaacttaagctgattaacaaatcagtgaaacagggtttcgaacgcagtgcgtacggaaaattccatcgacgcattagccggcttggtattgctagtaaggcgctatatgacaacgctgtgccgaaggaaggattcgtagaacatcctgaatatgctccagctctgcaaagggattatgcgttggcaggagagcgaattgaggcggttggtggaactttcaggttgcaaacacctcagcggaaatcggctcttgtagatcatatcttgagtatgcccgaaggtttcgatcaagctcatgcccttgagtctatagcccctcatgttggcgatctaagcgcggaagatcgccatcgtgttgtgaacaaaatgctcgagcactttgccaccccgtttcaagaaggggggctgccaaccgacgaaaatttttgtggaacgtacgctttgtccaaggcgtttcctcatatggaagacagcctaaaagcaaagatgctggatgtggtgttagagcatcctcacttagccaacctccattggacgcagagagaactagttgaagggtggcatcaagaccgtgaagctgagaagtcagcctggcgttccaatcaaaagccaattgtcgaggggcgacttgcagacctcgaacgatcgatccacagtgatatcgaggtgacaaacaggagtgattttgcgaggatgcgttcagccaggcccttgcaacaagctataagcgatacgttcgatcttgcgcgtgaggagctcaaaagccgccctcgtgaagggcggggtcgataattacatctattggtctttctgccgaacctctatgaaaatccgcacgtcaaatgtggcttttacttcccgatttcaattgaagcgctaagctgaaaatccgtccccatttctgacctaacggtttcatcaccggggcagggagcggcgcttaaaacggtgacggccaccaccaaaggacatgttcgctcacgcggtgcctccgtgcttctttgcaagagcctgcttcaggctgatgatctccttcagtgcgttggtgtgaatccgctccaggcgtttcagttcgctctccaaataggcgttgcgcttatccagcctcgtccgatcttcttccaagctctccattttcactagcgcgttatggtaattcaaccgtaactcctcaaaaaaagcacgttcggcgacgaaatgggtttcggaatcgagttcaggtatgtcgccgacatgcacgacgcgaaccgccttgccggcagcggcgagccgggcgcggtaagcgcgttgaatttccgcttgggtcagcgcgtttggcttgcggggccgcgaggaaatttgggagtaaccataacggttatttatcataacgcgttatcactggatagcatcggcgcaggcggatggcgcgcttcgccgggtcgaaatcaatatttccagatcgagatggattgaagacctgaagcccgttccatacagaagctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaacctgcgctcgttcgccaggccaggacagaaatgcctcgacttcgctgctgcccaaggttgccggggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaagtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcggccgcaaagcgctccggcttaactcaggcgttagtcccatcacccgctttatgcgcgcagccacttatttcagcgctccgccctgttccaaaggctttctagccttttgggcctccagcgcttgtgcagcaagcgccaccagctatccttcttgcagcgctgcgccgctttcgtggtccagcggtttttcttgggctgcgcccttcttcaaggccgggcgctcccttttggccttcgggtctaactctgcggtcaagcggacccgcattctgcgggccgcttaccttggccgttagacatcatgagggtagcggtgaccatcgaaatttcgaaccaactatcagaggtgctaagcgtcattgagcgccatctggaatcaacgttgctggccgtgcatttgtacggctccgcagtggatggcggcctgaagccatacagcgatattgatttgttggttactgtggccgtaaagctctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcagggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatcctttctcagagcccagacgccttggttaggttggtgcggaaccgatcccgccgccgctggtatttgcgcgtcatctcggccgaggcgtggcctagttgtttctgaacgtaacgctcctcaatgtcggccgaggaggcaagaccggcgcgcagcgagtgccctgagaacagcagcgcccgttcccttctggaaagatcggaacggacgccggcggcgagtgcggtctgcttgaccaggcgggcgacatgcttgtcggagagccgctcgacgtccacggtcttgttgtccttgaagatgcgccggaacaggggaccgcgcgcgatcctgccgaagcggatctgatcaccggcggttccgaagctcggtcccgtgggtgttctgtcgtctcgttgtacaacgaaatccattcccattccgcgctcaagatggcttcccctcggcagttcatcagggctaaatcaatctagccgacttgtccggtgaaatgggctgcactccaacagaaacaatcaaacaaacatacacagcgacttattcacacgagctcaa

SEQ ID NO: 34 - DNA, 9693 bp, synthetic construct
  misc_feature 1..9693: note = DNA Construct
  source 1..9693: mol_type = other DNA
cattgcacctatggtgttgccctggctggggtatgtcagtgatcgcagtagaatgtactaattgacaagttggagaatacggtagaacgtccttatccaacacagcctttatccctctccctgacgaggtttttgtcagtgtaatatttctttttgaactatccagcttagtaccgtacgggaaagtgactggtgtgcttatctttgaaatgttactttgggtttcggttctttaggttagtaagaaagcacttgtcttctcatacaaaggaaaacctgagacgtatcgcttacgaaagtagcaatgaaagaaaggtggtggttttaatcgctaccgcaaaaacgatggggtcgttttaattaacttctcctacgcaagcgtctaaacggacgttggggttttgctagtttctttagagaaaactagctaagtctttaatgttatcattagagatggcataaatataatacttgtgtctgctgataagatcattttaatttggacgattagacttgttgaactacaggttactgaatcacttgcgctaatcaacaagctgtaaagcttacgctattccgtggtctcaagttttagagctagaaatagcaagttaaaataaggctagtccgttatcaacttgaaaaagtggcaccgagtcggtgctttttttcctaagggatttaaggacgtgaactctgttgagatctctgtgaaattcagagggtgggtgataccatattcactgatgccattagcgacatctaaatagggctaattgtgactaatttgagggaatttcctttaccattgacgtcagtgtcgttggtagcatttgagtttcgtaactataacggtcctaaggtagcgaacatcttgttctggggtttcacactatctttagagaaagtgttaagttaattaagttatcttaattaagagcataattatactgatttgtctctcgttgatagagtctatcattctgttactaaaaatttgacaactcggtttgctgacctactggttactgtatcacttacccgagttaacgagatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtgagtcgaccgggcatgtcccgaagacattaaactacggttctttaagtagatccgtgtctgaagttttaggttcaatttaaacctacgagattgacattctcgactgatcttgattgatcggtaagtcttttgtaatttaattttctttttgattttattttaaattgttatctgtttctgtgtatagactgtttgagatcggcgtttggccgactcattgtcttaccataggggaacggactttgtttgtgttgttattttatttgtattttattaaaattctcaacgatctgaaaaagcctcgcggctaagagattgttggggggtgagtaagtacttttaaagtgatgatggttacaaaggcaaaaggggtaaaacccctcgcctacgtaagcgttattacgcccgtctgtacttatatcagtacactgacgagtccctaaaggacgaaacgggagaacgctagccaccaccaccaccaccacgtgtgaattacaggtgaccagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcctaggccaccatgttgggcccggcgcgccatcaacatgtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggatccggcgcgccataaaa

SEQ ID NO: 35 - DNA, 13106 bp, synthetic construct
  misc_feature 1..13106: note = DNA Construct
  source 1..13106: mol_type = other DNA
taacaagcttcgtctcagtcaggagcatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgccgatggtctttcatgcactgttttagagctagaaatagcaagttaaaataaggctagtccgttatcaacttgaaaaagtggcaccgagtcggtgctttttttctagacccagctttcttgtacaaagttggcattacgctgtcaggagcatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgtgcttcatccattagatctagttttagagctagaaatagcaagttaaaataaggctagtccgttatcaacttgaaaaagtggcaccgagtcggtgctttttttctagacccagctttcttgtacaaagttggcattacgctgtcaggagcatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgcccagtgcatgaaagaccatgttttagagctagaaatagcaagttaaaataaggctagtccgttatcaacttgaaaaagtggcaccgagtcggtgctttttttctagacccagctttcttgtacaaagttggcattacgctgtcaggagactagagccaagctgatctcctttgccccggagatcaccatggacgactttctctatctctacgatctaggaagaaagttcgacggagaaggtgacgataccatgttcaccaccgataatgagaagattagcctcttcaatttcagaaagaatgctgacccacagatggttagagaggcctacgcggcaggtctgatcaagacgatctacccgagtaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcataaaccaaggcaagtaatagagattggagtctctaagaaagtagttcctactgaatcaaaggccatggagtcaaaaattcagatcgaggatctaacagaactcgccgtgaagactggcgaacagttcatacagagtcttttacgactcaatgacaagaagaaaatcttcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggactccggtatttttacaacaataccacaacaaaacaaacaacaaacaacattacaatttactattctagtcgaaatggacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagactatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagattatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtctccacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtttcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaaggatctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgtgagcttggaatggatcttcgatcccgatcgttcaaacatttggcaataaagtttcttaagattgaatcctgttgccggtcttgcgacgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcdcggtgtcatctatgttactagatcgggaattgccaagctaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatgggaccgactcgcgcttgagacgaagctttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcatctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacctctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatccgatgagtaaataaacaaatacgcaaggggaacgcatgaaggttatcgctgtacttaaccagaaaggcgggtcaggcaagacgaccatcggaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatagatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggactctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggtgacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagtcgaaatacgcgatcgctgttaaaaggacaattacaaacaggaatcgaatgcaaccggcgcaggaacactgccagcgcatcaacaatattttcacctgaatcaggatattcttctaatacctggaatgctgttttccctgggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcggtagattgtcgcacctgattgcccgacattatcgcgagcccatttatacccatataaatcagcatccatgttggaatttaatcgcggccttgagcaagacgtttcccgttgaatatggctcataacagaacttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaaca

SEQ ID NO: 36 - DNA, 13272 bp, synthetic construct
  misc_feature 1..13272: note = DNA Construct
  source 1..13272: mol_type = other DNA
ggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacataacaagcttcgtctcagtcaggagcatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgccgatggtctttcatgcactgttttagagctagaaatagcaagttaaaataaggctagtccgttatcaacttgaaaaagtggcaccgagtcggtgctttttttctagacccagctttcttgtacaaagttggcattacgctgtcaggagcatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgtgcttcatccattagatctagttttagagctagaaatagcaagttaaaataaggctagtccgttatcaacttgaaaaagtggcaccgagtcggtgctttttttctagacccagctttcttgtacaaagttggcattacgctgtcaggagcatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgcccagtgcatgaaagaccatgttttagagctagaaatagcaagttaaaataaggctagtccgttatcaacttgaaaaagtggcaccgagtcggtgctttttttctagacccagctttcttgtacaaagttggcattacgctgtcaggagttctgagcctctttccttctaatccactcatctgcatcttcttgtgtccttactaatacctcattggttccaaattccctccctttaagcaccagctcgtttctgttcttccacagcctcccaagtatccaagggactaaagcctccacattcttcagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaagatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaatggacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagactatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagattatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtctccacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtttcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaaggatctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgtgagcttcggccatgctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctcgcttgagacgaagctttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcatctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacctctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatccgatgagtaaataaacaaatacgcaaggggaacgcatgaaggttatcgctgtacttaaccagaaaggcgggtcaggcaagacgaccatcggaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatagatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggactctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggtgacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagtcgaaatacgcgatcgctgttaaaaggacaattacaaacaggaatcgaatgcaaccggcgcaggaacactgccagcgcatcaacaatattttcacctgaatcaggatattcttctaatacctggaatgctgttttccctgggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcggtagattgtcgcacctgattgcccgacattatcgcgagcccatttatacccatataaatcagcatccatgttggaatttaatcgcggccttgagcaagacgtttcccgttgaatatggctcataacagaacttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcaca

SEQ ID NO: 37 - DNA, 13272 bp, synthetic construct
  misc_feature 1..13272: note = DNA Construct
  source 1..13272: mol_type = other DNA
ttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacataacaagcttcgtctcagtcaggagcatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgtattaatgagaattaatggtgttttagagctagaaatagcaagttaaaataaggctagtccgttatcaacttgaaaaagtggcaccgagtcggtgctttttttctagacccagctttcttgtacaaagttggcattacgctgtcaggagcatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgtttggctttctccaagaaatgttttagagctagaaatagcaagttaaaataaggctagtccgttatcaacttgaaaaagtggcaccgagtcggtgctttttttctagacccagctttcttgtacaaagttggcattacgctgtcaggagcatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgtatatgggtttgccatatttgttttagagctagaaatagcaagttaaaataaggctagtccgttatcaacttgaaaaagtggcaccgagtcggtgctttttttctagacccagctttcttgtacaaagttggcattacgctgtcaggagttctgagcctctttccttctaatccactcatctgcatcttcttgtgtccttactaatacctcattggttccaaattccctccctttaagcaccagctcgtttctgttcttccacagcctcccaagtatccaagggactaaagcctccacattcttcagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaagatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaatggacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagactatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagattatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtctccacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtttcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaaggatctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgtgagcttcggccatgctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctcgcttgagacgaagctttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcatctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacctctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatccgatgagtaaataaacaaatacgcaaggggaacgcatgaaggttatcgctgtacttaaccagaaaggcgggtcaggcaagacgaccatcggaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatagatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggactctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggtgacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagtcgaaatacgcgatcgctgttaaaaggacaattacaaacaggaatcgaatgcaaccggcgcaggaacactgccagcgcatcaacaatattttcacctgaatcaggatattcttctaatacctggaatgctgttttccctgggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcggtagattgtcgcacctgattgcccgacattatcgcgagcccatttatacccatataaatcagcatccatgttggaatttaatcgcggccttgagcaagacgtttcccgttgaatatggctcataacagaacttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcg

SEQ ID NO: 38 - DNA, 3097 bp, synthetic construct
  misc_feature 1..3097: note = DNA Construct
  source 1..3097: mol_type = other DNA
cagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtaccgggccccccctcgaggtcgatttgtaatacgactcactataggtgcttcatccattagatctagttttagagctagaaatagcaagttaaaataaggctagtccgttatcaacttgaaaaagtggcaccgagtcggtgctttttttcctaagggatttaaggacgtgaactctgttgagatctctcgaggtaccggatccactagttctagagcggccgccaccgcggtggagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttac

(19) United States
(12) Patent Application Publication
(10) Pub. No.: US 2024/0381898 A1
(43) Pub. Date: Nov. 21, 2024

(54) EDIBLE COMPOSITION COMPRISING NON-TOXIC SOLANUM PLANT OR PLANT PART

(71) Applicant: RUMAFEED LTD., Mishmar HaShiva (IL)

(72) Inventor: Haim D. RABINOWITCH, Kiryat Ono (IL)

(21) Appl. No.: 18/689,119

(22) PCT Filed: Sep. 5, 2022
(86) PCT No.: PCT/IL2022/050969

§ 371 Date: Mar. 5, 2024

(30) Foreign Application Priority Data
Sep. 5, 2021 (IL) 286181

(51) Int. Cl. A23K 10/37; A23K 10/35; A23K 50/10; C12N 15/82

(52) U.S. Cl. CPC A23K 10/37; A23K 10/35; A23K 50/10; C12N 15/8243

(57) ABSTRACT

The present invention is directed to an edible composition including a plant belonging to the genus Solanum or plant part derived therefrom, wherein the plant or plant part is essentially devoid of steroidal glycoalkaloids (SGAs), and wherein the in vitro dry matter digestibility (IVDMD) of the plant or plant part is greater than or essentially equivalent to the IVDMD of a control plant belonging to the genus Solanum or plant part comprising SGAs. Further provided are a genetically modified plant belonging to the genus Solanum characterized by being essentially devoid of SGAs and having IVDMD greater than or essentially equivalent to the IVDMD of a control plant, and a method for producing same.

### CROSS-REFERENCE TO RELATED APPLICATIONS

This application claims the benefit or priority of Israel Patent Application No. 286181, titled “LIVESTOCK FEED COMPRISING NON-TOXIC SOLANACEAE PLANT TISSUES”, filed 5 Sep. 2021, the contents of which are incorporated herein by reference in their entirety.

### FIELD OF THE INVENTION

The present invention is in the field of sustainable agriculture, providing edible compositions, including feed compositions for animals, including, but not limited to livestock, comprising non-toxic haulm of a solanum plant or plant part derived therefore, such as potato haulm, and reducing the need for waste management in crop, e.g., potato, production.

### BACKGROUND OF THE INVENTION

A worldwide increase of human population has enhanced the demand for meat, milk, eggs, and other foods production in order to improve food security. As a result, the demand for fodder to feed livestock has increased accordingly. For example, in 2017, 1.6 billion tons of fodder were used globally to produce meat, eggs, and milk, and the demand will increase as livestock production intensifies. However, since many livestock diets include raw materials that could be eaten directly by humans, such as cereal grains, a debate about the competition between livestock and humans for land and other resources has evolved. Currently, an attempt to resolve this matter is aimed towards a more sustainable livestock management based, inter alia, on the efficient use of available food resources.

Food waste is a matter intrinsically linked to food security. Globally, an estimated 1.3 billion tons of food for humans is lost and wasted each year, enough to feed more than one billion people. Food waste is also a resource and sustainability issue.

Recovering food waste for animal feeding (ReFeed) is a viable option that has the potential to simultaneously address waste management, food security, and resource and environmental challenges. Livestock animals function as bio-processors for converting food materials that are either unpalatable/inedible or no-longer-wanted by humans into meat, eggs, and milk. This would concomitantly ‘spare’ feed grains and relevant resources and environmental burdens associated with the production of the feed grains.

Potato (Solanum tuberosum) residual wastes such as potato peel are one of the prominent food wastes that are used as alternative animal feed due to natural sources of energy, fiber, and protein. Potato is the fourth most important crop in the world. The tubers are grown in 120 countries on an area of 200 million dunams, and the global yield of potato tubers is estimated at 400 million tons. Above-ground parts of potato (haulms) are another type of residual waste which are destroyed by the growers about 10 days before harvesting with an aim to allow the ripening of the tubers and prevent contamination of the produce in remnants of foliage. Currently, the destruction of the haulm is carried out with herbicides (some of them are banned for use of in the European Community) or by mechanical mowing. The remnants of the haulm dry up in the field and might serve as an infectious agent for pathogens in the next crop cycle. The damage can be reduced by concealing the remnants after the harvest, thus slightly enriching the soil with organic matter.

According to previous publications, potato haulm can be ensilaged and used as animal feed, demonstrating a quite close nutritional value to maize silage with high protein and low fiber content. Feeding ruminants with potato haulm has the potential to increase agricultural income and discharge vast areas of the world for growing other crops such as corn that will be used as food for humans instead of livestock feeding (45% of all food is given to ruminants, especially cows, sheep, and pigs).

However, steroidal glycoalkaloids (SGAs) content is the most critical issue to be considered in the use of potato haulm as a feed source. SGAs are typically found in plants from the Solanaceae family such as tomatoes and potatoes. Consisting of a C-27 cholestane skeleton and a heterocyclic nitrogen component, SGAs were suggested to be synthesized in the cytosol from cholesterol. The oligosaccharide moiety components of SGAs include D-glucose, D-galactose, L-rhamnose, D-xylose, and L-arabinose, the first two monosaccharide being the predominant units directly conjugated to the hydroxyl group at C-3β of the alkamine steroidal skeleton (aglycone). Although several optional pathways for SGA biosynthesis were suggested, the complex network of their biosynthesis was not elucidated to date.

Potato is known to contain the SGAs in nearly all potato tissues. The principal glycoalkaloids are α-chaconine (solanidine-glucose-rhamnose-rhamnose) and α-solanine (solanidinegalactose-glucose-rhamnose), which generally contribute about 90-95% total glycoalkaloids (TGAs). Other glycoalkaloids that occur in smaller quantities include β-chaconine, γ-chaconine, β1-solanine, β2-solanine and γ-solanine.

In plants, SGAs serve as phytoanticipins (antimicrobial compounds) that provide a pre-existing chemical barrier that protects plants against a broad range of pathogens using mechanisms of toxicity that include the disruption of membranes and the inhibition of acetylcholine esterase activity. Sprouting potato tubers or other above-ground parts may be used as a source of food, and thus might expose animals and humans to relatively high levels of SGAs. Above certain levels (total SGAs levels must not exceed 20 mg per 100 g fresh weight in new potato cultivars), SGAs are known to be toxic to fungi, bacteria, insects, animals, and humans. Available information suggests that oral doses of SGAs in the range of 1-5 mg/Kg body weight are marginally to severely toxic whereas 3-6 mg/Kg body weight can be lethal. Although generation of SGA-free potato has not been achieved yet, many attempts were made to control and attenuate the SGAs levels in potato plant.

International Patent Application Publication No. WO 2012/095843 provides means and methods to modulate GLYCOALKALOID METABOLISM 4 (GAME4), a member of the Cytochrome P450 subfamily CYP88B1 and a key enzyme in the cytosolic mevalonic acid isoprenoid biosynthetic pathway, which leads, inter alia, to the production of SGAs. Genetically modified plants, in which the expression of GAME4 has been modified, either inhibited or enhanced, showed essentially the same growth pattern compared to corresponding wild type plants.

In another study, an additional key enzyme designated St16DOX active in the SGAs biosynthesis and a member of the 2-oxoglutarate-dependent dioxygenase (2OGD) superfamily (the second largest enzyme family, following the CYP superfamily, in the plant genome) was identified and characterized. The 16DOX gene was coexpressed with the previously identified SGA biosynthetic genes in potato and the 16DOX protein was found to catalyze the hydroxylation of cholesterol at the C-16a position. Furthermore, 16DOX silencing in transgenic potato plants led to significantly reduced endogenous SGA without affecting potato tuber yield, indicating that 16DOX may be a suitable target for controlling toxic SGA levels in potato.

In recent years, various gene editing technologies have been applied to induce site-directed mutagenesis in solanaceous food crops. Genome edited plants using novel technologies like Clustered Regulatory Interspaced Short Palindromic Repeat (CRISPR) and CRISPR-Associated protein 9 (Cas9) system (CRISPR/Cas9) or Transcriptional Activator-Like Effector Nucleases (TALEN), are differentiated from conventional transgenic plants as they may not incorporate foreign DNA. Although genome editing can be used to introduce foreign DNA into the genome, it may simply involve changes of a few base pairs in the plant's own DNA. This distinction makes genome editing a novel and powerful breeding tool that has promising applications in agriculture, especially when genome edited crops are not regulated as genetically modified (GM). In potato, a gene named sterol side chain reductase 2 (SSR2) that is committed to cholesterol biosynthesis and involved in SGA production was disrupted by TALEN using Agrobacterium tumefaciens-mediated stable transformation system and resulted in a significant decrease in the SGA content in the potato plants. Another study reported that knockout of 16DOX by using CRISPR/Cas9 caused a complete abolition of the SGA accumulation in potato hairy roots.

There remains an unmet need for a commercial scale growing of non-transgenic potato plants, the haulm and tuber of which are non-toxic, containing low SGA levels, which enable the utilization of the raw potato waste, specifically use of post-harvest haulm as a feed with high nutritional values for ruminant animals.

### SUMMARY OF THE INVENTION

The present invention, in some embodiments, relates to the field of sustainable agricultural, combining environmental considerations in treating solanum plant, e.g., potato post-harvest waste management and the growing needs for nutritional feed for ruminant farm animals.

The present invention, in some embodiments, utilizes the potato haulm, hitherto treated as a waste due to a toxic content of SGAs, as feed having high digestibility and nutritional values. The feed of the invention is based on potato haulm essentially devoid of SGAs. Advantageously, according to some embodiments of the invention, the haulm cells are devoid of heterologous polynucleotides, and are thus non-transgenic.

In some embodiments, the present invention provides an animal feed comprising potato haulm, wherein the haulm is essentially devoid of steroidal glycoalkaloids (SGAs), and wherein the in vitro dry matter digestibility (IVDMD) of the haulm is essentially equivalent to the IVDMD of a wild type (WT) potato haulm comprising SGAs.

According to one aspect, there is provided an edible composition comprising a plant belonging to the genus Solanum or plant part derived therefrom, wherein the plant or plant part is essentially devoid of steroidal glycoalkaloids (SGAs), and wherein the in vitro dry matter digestibility (IVDMD) of the plant or plant part is greater than or essentially equivalent to the IVDMD of a control plant belonging to the genus Solanum or plant part comprising SGAs.

According to another aspect, there is provided a genetically modified plant belonging to the genus Solanum characterized by being essentially devoid of SGAs and having IVDMD greater than or essentially equivalent to the IVDMD of a control plant, the genetically modified plant comprising at least one enzyme being selected from the group consisting of: 16DOX, GAME4, and both, being characterized by having reduced expression and/or activity compared to the expression and/or activity of the at least one enzyme in a control plant.

According to another aspect, there is provided a method for producing a plant or plant part being essentially devoid of SGAs, and having an IVDMD being greater than or essentially equivalent to the IVDMD of a control plant, the method comprising mutating at least one gene encoding at least one enzyme being selected from the group consisting of: 16DOX, GAME4, and both, such that the at least one enzyme is characterized by having reduced expression and/or activity compared to the expression and/or activity of the at least one enzyme in a control plant or plant part.

In some embodiments, the plant or plant part comprises a protein content of 35-50% weight per weight of dry matter of the plant or plant part.

In some embodiments, the plant or plant part comprises potassium in an amount of 3-10% weight per weight of dry matter of the plant or plant part.

In some embodiments, the plant or plant part comprises at least one essential amino acid being selected from the group consisting of: Histidine, Isoleucine, Leucine, Lysine, Phenylalanine, Threonine, Valine, and any combination thereof, in an amount being at least 10% by weight greater that in a control plant.

In some embodiments, the plant belonging to the genus Solanum is selected from the group consisting of: potato, tomato, and eggplant.

In some embodiments, the plant part comprises haulm.

In some embodiments, the haulm comprises from 0 to 0.25 mg SGAs per 100 g fresh weight (FW).

In some embodiments, the IVDMD of the haulm is greater than or essentially equivalent to the IVDMD of a standard hay or silage feed.

In some embodiments, the plant or plant part is genetically modified such that at least one enzyme being selected from the group consisting of: 2-oxoglutarate-dependent dioxygenase (16DOX), glycoalkaloid metabolism 4 (GAME4), and both, is characterized by having reduced expression and/or activity compared to the expression and/or activity of the at least one enzyme in a control plant or plant part.

In some embodiments, the genetically modified plant or plant part comprises a mutation in Exon 1 of a gene encoding the DOX16 enzyme.

In some embodiments, the genetically modified plant or plant part comprises a mutation in Exon 1, Exon 3, or both, of a gene encoding the GAME4 enzyme.

In some embodiments, the mutation comprises a deletion or insertion.

In some embodiments, any one of the insertion and deletion is of 1 to 15 nucleotides.

In some embodiments, the mutation is introduced into an exon of a gene using a programmable engineered nuclease (PEN).

In some embodiments, the PEN is a clustered regularly interspaced short palindromic repeat (CRISPR) type II system a gene-editing method.

In some embodiments, the plant or plant part is devoid of heterologous polynucleotides.

In some embodiments, the control plant comprises a wild type (WT) plant belonging to the genus Solanum.

In some embodiments, the edible composition is for use in feeding of a mammal subject.

In some embodiments, the mammal is a livestock animal.

In some embodiments, the livestock animal a ruminant farm animal.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A-1D include illustrations showing the construction of plasmids used. (1A) Plasmid map 4821 with hCas9 and 3 U6 guides for S. tuberosum GAME4. (1B) Plasmid map 4823 with Ubiq-hCas9 and 3 U6 guides for S. tuberosum DOX16. (1C) Plasmid map 4728 with a T7 promoter for in vitro transcription of the sgRNA for S. tuberosum DOX16 Exon 2 site 25. (1D) Plasmid map 4794 with 35S-hCas9 and 3 U6 guides for S. tuberosum DOX16.

FIGS. 2A-2B include illustrations showing the maps of the binary plasmid constructs. (2A) Plasmid 4064 with Cas9 expression cassette. (2B) Plasmid 1453 with mGUS transient QQR target site construct.

FIGS. 3A-3B include illustrations showing tobacco rattle virus TRV1-TRV2 components. (3A) Map of TRV1. (3B) Map of TRV2.

FIGS. 4A-4D include illustrations showing 4 various guide RNAs cloned to TRV2 vectors under sub-genomic promoter, creating the 4 viral constructs. (4A) Plasmid 8065 a TRV2 with sgRNA of DOX16 Ex1-79. (4B) Plasmid 4906 a TRV2 with sgRNA of DOX16 Ex2-25. (4C) Plasmid 8041 a TRV2 with sgRNA of SSR2 52. (4D) Plasmid 8042 a TRV2 with sgRNA of SSR2 631.

FIG. 5 includes a vertical bar graph showing a comparison of in vitro digestibility between low-SGA mutated lines to commonly-used hay for ruminants.

### DETAILED DESCRIPTION OF THE INVENTION

The present invention answers the need of converting an “agricultural waste”, e.g., potato haulm, to a useful product, at an agricultural economic cost. In fact, the teachings of the present invention enable using the common practices of potato cultivation, with the harvested haulm taken to be used as a livestock feed, thus eliminating the costly and potentially environmentally hazardous haulm waste management.

According to some embodiments, there is provided an edible composition comprising a plant belonging to the genus Solanum or plant part derived therefrom, wherein the plant or plant part is essentially devoid of steroidal glycoalkaloids (SGAs), and wherein the in vitro dry matter digestibility (IVDMD) of the plant or plant part is greater than or essentially equivalent to the IVDMD of a control plant.

The IVDMD of a control plant, such as, a standard wheat or legume hay used as feed, is typically from about 50% to about 60%. The IVDMD of standard silage (wheat, corn, barley and/or legume) used as feed is typically from about 60% to about 70%.

In some embodiments, the IVDMD of the plant or plant part of the invention is from about 60% to about 65%, about 60% to about 70%, about 60% to about 75%, or about 65% to about 70%. Accordingly, the IVDMD of the plant or part thereof of the invention is at least equivalent to the IVDMD of standard hay or silage feed. According to some embodiments, the IVDMD of the plant or part thereof of the present invention is greater than the IVDMD of a control, e.g., a standard hay or silage feed.

According to some embodiments, there is provided a haulm essentially devoid of steroidal glycoalkaloids (SGAs) that is genetically modified to have reduced expression and/or activity of at least one enzyme involved in the biosynthesis of SGAs in potato haulm cells compared to the expression and/or activity of the at least one enzyme in a corresponding unmodified potato haulm.

According to some embodiments, the plant or plant part, such as a haulm, comprises less than 0.5 mg SGAs per 100 g fresh weight (FW) of the plant or plant part, e.g., haulm. According to some embodiments, the plant or plant part, such as haulm, comprises less than 0.25 mg, less than 0.1 mg, or less than 50 μg SGAs per 100 g FW. According to some embodiments, the plant or plant part, e.g., haulm comprises, from 0 to 5 μg SGAs per 100 g FW of the plant or plant part as disclosed herein.

In some embodiments, the term “essentially devoid of SGAs” comprises less than 0.25 mg, less than 0.1 mg, or less than 50 μg SGAs per 100 g FW. In some embodiments, the term “essentially devoid of SGAs” refers to comprising from 0 to 5 μg SGAs per 100 g FW of the plant or plant part as disclosed herein.

In some embodiments, the control plant belongs to the genus Solanum or plant part comprising SGAs.

In some embodiments, the plant or plant part comprises a protein content of 32-45%, 35-48%, 38-49%, 37-47%, 39-50%, or 35-50% weight per weight (w/w) of dry matter of the plant or plant part. Each possibility represents a separate embodiment of the invention.

In some embodiments, the plant or plant part comprises a protein content being at least 10%, at least 15%, at least 20%, at least 25%, at least 30% greater than the protein content of a control plant, or any value and range therebetween. Each possibility represents a separate embodiment of the invention. In some embodiments, the plant or plant part comprises a protein content being 10-40%, 15-35%, 20-38%, 25-40%, or 30-45% greater than the protein content of a control plant. Each possibility represents a separate embodiment of the invention.

In some embodiments, the plant or plant part comprises potassium in an amount of 1-10%, 2-10%, 3-10%, 2-8%, 5-9%, or 3-8% weight per weight (w/w) of dry matter of the plant or plant part. Each possibility represents a separate embodiment of the invention.

In some embodiments, the plant or plant part comprises potassium in an amount being at least 40%, at least 50%, at least 60%, at least 70%, at least 80% greater than the amount of potassium in a control plant, or any value and range therebetween. Each possibility represents a separate embodiment of the invention. In some embodiments, the plant or plant part comprises potassium in an amount being 40-90%, 50-85%, 65-85%, 50-90%, or 70-90% greater than the amount of potassium in a control plant. Each possibility represents a separate embodiment of the invention.

In some embodiments, the plant or plant part comprises at least one essential amino acid being selected from: Histidine, Isoleucine, Leucine, Lysine, Phenylalanine, Threonine, Valine, or any combination thereof, in an amount being at least 5%, at least 10%, at least 15%, at least 20%, at least 25%, at least 30%, at least 35%, at least 40%, at least 45%, at least 50%, at least 60% by weight greater that in a control plant. Each possibility represents a separate embodiment of the invention. In some embodiments, the plant or plant part comprises at least one essential amino acid being selected from: Histidine, Isoleucine, Leucine, Lysine, Phenylalanine, Threonine, Valine, or any combination thereof, in an amount being 20-80%, 30-75%, 35-65%, 40-60%, or 40-70% greater than the amount of the at least one essential amino acid as disclosed in a control plant. Each possibility represents a separate embodiment of the invention.

In some embodiments, the amount of an amino acid, e.g., an essential amino acid, as disclosed herein, is defined in mg/g of dry matter.

In some embodiments, a plant as disclosed herein, belong or classified to the genus Solanum.

In some embodiments, a plant belonging or classified to the genus Solanum is selected from: potato, tomato, and eggplant. Lines, strains, species, and the like, of plants belonging or classified to the genus Solanum are common and would be apparent to one of ordinary skill in the art.

In some embodiments, a plant part comprises haulm. In some embodiments, a plant part comprises the green parts of a plant as disclosed herein. In some embodiments, a plant part comprises the foliage of a plant as disclosed herein. In some embodiments, a plant part comprises the ‘above ground’ parts of a plant as disclosed herein.

In some embodiments, ‘haulm’ comprises any one of: green parts, foliage, ‘above-ground’ parts' or any combination thereof, of a plant as disclosed herein.

In some embodiments, the plant part, such as haulm, comprises SGAs in an amount of less than 5 μg, less than 50 μg, less than 250 μg, less than 500 μg, less than 750 μg, or less than 1 mg, per 100 mg fresh weight (FW) of the plant or plant part as disclosed herein, or any value and range therebetween. Each possibility represents a separate embodiment of the invention. In some embodiments, the plant part, such as haulm, comprises SGAs in an amount of 0 to 1 mg, 0 to 900 μg, 0 to 800 μg, 0 to 700 μg, 0 to 600 μg, 0 to 400 μg, 0 to 300 μg, 0 to 200 μg, 0 to 100 μg, 0 to 90 μg, 0 to 70 μg, 0 to 50 μg, 0 to 35 μg, 0 to 20 μg, 0 to 15 μg, 0 to 10 μg, or 0 to 5 μg, per 100 mg fresh weight (FW) of the plant or plant part as disclosed herein. Each possibility represents a separate embodiment of the invention.

In some embodiments, IVDMD of the plant or plant part, e.g., haulm is greater than or essentially equivalent to the IVDMD of a standard hay or silage feed.

In some embodiments, the term “essentially equivalent” comprises having a difference of not more the 1%, 5%, 10%, 15%, or 20%, or any value and range therebetween. Each possibility represents a separate embodiment of the invention. In some embodiments, essentially equivalent comprises having a difference of 1-5%, 1-10%, 1-15%, 1-20%, 5-20%, 10-20%, or 15-20%. Each possibility represents a separate embodiment of the invention.

In some embodiments, IVDMD comprises IVDMD being determined up to or for about 48 hours (IVDMD48).

In some embodiments, the plant or plant part is a genetically modified plant or part derived therefrom.

In some embodiments, the genetically modified plant comprises or is characterized by having at least one enzyme being selected from: 2-oxoglutarate-dependent dioxygenase (16DOX), glycoalkaloid metabolism 4 (GAME4), or both, having reduced expression and/or activity compared to the expression and/or activity of the at least one enzyme in a control plant or plant part.

In some embodiments, expression comprises: gene expression, mRNA transcription, protein translation and/or modification, or any combination thereof.

In some embodiments, the genetically modified plant or plant part comprises a mutation in Exon 1 of a gene encoding the DOX16 enzyme. In some embodiments, the mutation is in nucleotide at position 79 of Exon 1 of the gene encoding DOX16 enzyme. In some embodiments, the mutation is in nucleotide at position 79 of Exon 1 of the gene encoding DOX16 enzyme in a potato or in an equivalent position in an ortholog DOX16 encoding gene in a plant belonging to the Solanum genus.

In some embodiments, the plant is a tomato plant or an eggplant plant, and the mutation is in a nucleotide located in Exon 1 of the DOX16 enzyme encoding gene in a position equivalent or homologous to position 79 of Exon 1 in the potato DOX16 enzyme encoding gene (SEQ ID NO:1).

In some embodiments. DOX 16 enzyme encoding gene of a potato comprises the nucleic acid sequence:

(SEQ ID NO: 1)
TACATATTATTTTGTAGGATTATAAAAAAGGTTACAACAATATATGTGTA
AGATTATATCAATATTTAATATATTTCTTATTTATCTATGTAAAATATTG
ATTGAGAAATTTGAATATGTATATTTTAATTTAATAAAAAATAAAATTAA
AATTACCCAAATGCGAATTGTTTTGTTTTATAAAAAAGTACTTTTCTGAA
AAATTATTTTAAAAAGTGTATCTAAAAAAAAGAAGGTAATTTCCAGTGAC
AATTGCCTAACAAGCTCTAAAAAATCTTAGCATAAATTTTGAATTTTTAG
TAGAATTTCTATACCCTTGGAGGACTTTGTTATGCAATTTTCTTCCCTAT
AAATGGCCCTCCATAGCTCAAATGAGATATCAGACAATTTAAAGAAGTAC
TATTAACATTTAGAAGATTTCTTTCTTTCCCAGGTAAATAAATCATTTTC
CCTCTTTCCTTCTTGCTCTTTCTTTGTTTATTTGTTCAGATTTTTACCCT
TTTTGTTTTGGTTAGATTCATTGACAATGGCGGACCTTCTTTCAAACTGG
TCAAGCACATTAGAAGCAGTTCCTCCAAGTCATTGCATCCCAGTGCATGA
AAGACCATCGGATCCAGTTGAAATTGTGGACAATATTCCAGTCATTGATT
TGGGAAAAGCTAATGGTGAAGAACGAAGTGTTGTTGTTAAAGAACTTTTG
AAAGCTTTTGAAGAATATGGGTTTTTTCAGGTTTATTATTTATACAATAG
TACAACTCTGTTCTTTTTTCTTTTTTTTTCTTATTGTATTTAAAAATGAT
CTGAAATTGAAATGATGAAATAGATAATCAATCATGGAGTACCCGTAGAT
CTAATGGATGAAGCAATGAAAGTGTACAAAGAATTTTTCAGTCTGCCAGC
AGCAGAGAAAGCAGAATATGCAAAGGATGCAGCTAATGATACAAATAGGG
GTGCAGCTACACTGTACAGTAGCAGCGCTAAGCATTATGATTCAGAGGAG
CATCGTTACTGGAGAGATGTCTTGGAACATAGCTGCAATCTTGATGGGAA
AGACAAAAAAACTTGGCCTAGTAACCCTCCAAGATATAGGTACCTACCTA
AACTATGCTTAGCAAAATTCCCTCTTGTTATTTTTCTTACCTAGTATTTG
CTTGTCCTTCAGGGAGGTTATTGGTGCATATGGAGATGAATTGAGAAGGG
TGAGCAAAGTTATCTTGGGTCTGTTAGCTGAAGGGCTAGGTTTGGAGGCA
GGGTTCTTTGACACAGAACTTGGGCAGAGAATGCTTGTGAATCACTATCC
AGCATGCCCAGATCCAAGTTTAACCTTGGGAGTTGGTGGACATTGTGATC
CTAATCTCATAACCATTATCCAACAAGAAGTGTATGGTCTTCAAATATTG
AAGGATGACAAATGGATTGGTGTGCAGCCTATCCGCAATGCATTTGTGGT
CAATTCTGGTTTACCAATTACGGTAGGTGTAACACTTTCTCTTAATTTTC
ATGGTCTACAAGCGATTCTCTTATTGCTCTGTTTTTTTTGTATAAATACA
GGTAGTTAGCAATGGAAAGCTAACTAGTGTTGCACATCGTGTGGTGACAA
ACACAACTCATTCACGAACCTCCATTGGTACTTTTATTTGCCCACACGAT
ATTGTTGAGCCTGCAAAAGCACTTGTTGGTCCGGAGAATCCTCCACAGTT
CAAATCCTTTAATTGGGGAATTGATTTTATGCCACATTACCTCAGCAAGA
AATCAGTTTACCACGCATCATTGGAGCCCTTCAAAATCGATGCTTAA,
or a homolog or an ortholog thereof, having at
least 70-100% homology thereto.

The sequence of DOX16 enzyme encoding gene is known and would be apparent to one of ordinary skill in the art, such as from PubMed. Further, sequence alignment of DOX16 enzyme encoding genes can easily be employed by a skilled artisan so as to determine a nucleotide being at a position equivalent or homologous to position 79 of Exon 1 of the potato DOX16 enzyme encoding gene.

In some embodiments, GAME4 enzyme encoding gene of a potato comprises the acid nucleic sequence

(SEQ ID NO: 2)
ATAACGTAAATTTTCAAAAGAGTGGAGGATGATTGAAAAAAACTCAAACA
TATCATCAAACTTTGAGAAAATACTCATTTATGTCGTTCGTTAAAAGTTTG
GCTCAATATTTATGTCATTATTTGTTAACTGAAACGACATAGATGAACCAA
AATTTTAACAGATGACATGAATAAATCTTTTCTCAAAGTTCGATAACATAT
TTGAACCTTTTTCCTTTTAAAAAATAATTTAATTAATGACATGGCCGAAAC
ATAATTGATCATATATAAAAATAAATAAATTAGTTTACCAATAATGAAACC
TTTATATTAACCAAAATTTGGGGGGCTAAATGATTTTCACGTGATTGTATT
GTTTTTTTATTGCACATTTATTTGGGAATAATAAAGATTTAACTATATTTGA
AAGGAAAAAGGTTAATATGGTCTTTTTACATATTTGGTCATCATTATTGTT
GCCATATCTTTGGATATAAATAAATAAATGTAGCCATTTAGCACCTCCAAT
CAAATTCACAGTCATCCAAATTATTCTAAGAAAAAGAGAGAGAGAGAACA
ATGGATTTCTACAATTTAGCCTTATTCTTCATAGCTTTAGTAATTGGGATTT
TCACATTTTATGCTATATTAATGAGAATTAATGGTTGGTATTATGCAATCA
AATTTTGTTCAAAGAAATATAACATCCCTCTTGGTTATATGGGTTTGCCAT
ATTTTGGCAACACACTTTCTTACTTCAAATCTACCATTTGTGGTGATCCAAA
TTCATTCCTTGATTTCTTTGCTACTAGGTAAATTAACTATTTTCATTATCGT
ACTTATTTGCTATGTTGTTTGAATTCTTGAAAAATATTAATATGTACTTGTC
AAATCTTTTAAAAATAGTGCATTTTTGAAGAATCTAACATGAGTACTGCAA
CTGTTATTACAATTACATTTTTGTAGAGTCCAATTGAACAAAATTTTTCTTT
TTTTTTTTAAAGGTTTGGGACAGGAGGAATGTATAGGGCATACATATTTGG
GAAGCCAACAATTATGGTGACAAAGCCAGAAATAATTAGAAAAGTTTTGA
TGGATGAAGAATATCTTGAAAGAGGTTTGCCTAATTATATGAAAAAATTA
ATTGGATTAACAACTTCGATTGAAGAAGATAAATATTTTCGTCGATTAACA
TCTCCAGTAAAAAGTCATGGATTATTATCCGATTATTTTGATTATATCGATA
AAACTGTGAGCACTACATTAGAGAAATACGCTACTACGGAAGAACCTATT
GAGTTTCTCCATAAGATGCACAGGCTTGCATTTGAGGTGTTTATGAGACTT
CTTATTGGTGATGAGGTTAATCAAGAATTTTTTGATCAAATGTTTGTGGAG
ATTACTGCTGTAATTAGTGCTGTTCACAACTTGCCAATTAATCTCCCAGGA
TTTCCTTATCATAAGGGACTCAAGGTAAGATGTGTTCAACTTTTAATATTAT
TTTTATTTCATTTTAAATTTTAAGATATAAATGTGTTTTTTTTAACTTGCCAT
CAGTTTATATTTGTGTACTTCAATTTTGAGTGTGTATAAACTTGAATTAGTA
GACACATGACATAATACACGTAGGACATCATGTAGGATGCAAATTGTCAC
GAAGGACGTGTGTGTTTACTTGTTCAACTTTATATAAGTTTAAGTGTTTACT
TATTCAACTTTATATAAGTTTAAGTGTTTACTTGTGCGTGTCCAAAGTTGAA
GGGTACACGTGTGAGATGAGGCCAAGTAAAATGGCATATTTATGTATTTTA
GATAGAATAACATGTTTTTTTTTTATATACATAATATATAAATGTGCCCTTC
GATTTGGATTCAACTGACATATATGCCCTCCAACTTTAGGTGTGCACAAAC
AGATGCTTAAACATATATAAAATTGAACAAATAGACACATTTGTCTTAAGT
GGCGTACATGACAATTTTGTGTCCTACGTGATGCCCTACATGTATTATGTT
ACGCAAGACACGCAAGACACGTGTGTCTACTTGTTTAATTTTATACAAGTT
TAAGTGTCTATTTGTACACACCCAAAATTAGTGGGCATAGATATTAGTTGA
AGCCAAGTTAAATGACATATTTATGTATTATATATTTTCTACACATGTAAT
GTGGGACCCCCCACCCNNNNNNNNNNNNNNNNNNNNNNNNNNNNNNNNN
NNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNN
NNNNNNNNNNNNNNNNNNNTACCAGCACCCCCCCACCCAAAAAAAATAA
TTTAAGTTCGTTTTTAAAAAATACTTTCAACTTCAAAATTTTATTTTTTCAC
CCCAACCCTCGACCCCCACCTATCCCGCCCCTACCAGCCCCCACCCCCCAA
AATAATTTAAGTTTATTTTTAAAAAATATTTTCAGTTTCAATTCAAAAAATA
TTTTCTACTCTCTAGTAAGAATAAAAGATATTTTCTCAAAAAAAAAATTCA
TTCATAAATCAAACACATAAAAATCTTTTTTCGAAAAATATTTTATACTCA
CCAACCAAACATGAGAAAATAAGTCCAAAGTCTACTTATTTTCTAGAAAA
ACATTTTCCTTCCTACCGAACACACCCAAAATCACCCCTTTCATCTTTTATC
ATGATATTTGTGGTTGTAATAAATATAATATAGAGTGCAACATTCATGTCA
AAGACCAAAAAGTACGAGATCACTCTTTTCATTTAATATTTATCGTGTTGT
ATATGTTTATTATGATTTCAGGCTCGAAAAGTACTAGGAGGGATATTTCAA
AAACTAATAGATGAAAGAAGAGAAGCCATGAAGGATGGAAAATCAATGC
CAAGGGCAAACATAATTGATATGTTGTTATCAAACACTAATCAAGATTATG
AAGCCAATATATTGAGTGACAAGAAGATCGTTGAAATCCTAGTTTTGTTTT
CATTTGCTGGTTTTGAACCTGTTGCTCTTATGTCTGTCAAGGCAATTTTTCA
CTTGCAAAAGCATCCCCATTTCTTGGAGAAAGCCAAAGTAAGTACTCTTTA
TTCTGTTTTAGGGGTGTCATATGGGTGAGTTAAACTGAATTTGGACAGGTG
GACTGAGTTAATTAGTCCAAAACTTACTTGGACTAAAATGGACTAACAAA
CGAGTCATAAACTCAACTCGTCTAATTAGACAGGTTGAGTTAAATTTGGAC
GAATTAAATTGGGCTAAGTTAATATGTCCAAAAATTTGGGGCTAAAATGA
GATAACAAAAGGATCATAACTCAACTCGTCTAATTGGTCGATCCAAACCTA
AGCGGGTTGGGTGGGTAGTGTATTCACGAGTGGATTTGCCACCCCTAGTTT
ATTTTATTTTATATGACGATATTTGATTAATTATTTTTTTTAACAGGAGGAA
CAAGAGGAAATAGTAAAGAGAAGAGCATCTTCAAATGCTGGACTTAGTTT
TGATGAGATTAGGCAAATGACGTTTGTTAGTAAGGTAAGACAATATTATG
ATGTTATATACTTTCTATAATAGCATTTTTATAAATAATACCATTATACAAA
AAGTCATCAGTACAAATAATTAAAAAAAGAAGATGAAGTGATAATACAAA
AATTGGACTAACATGCATTATTATTTATTAATGTTATCTCTTTTAATATTGA
CAGGTAATTAATGAAACGTTACGTATTGCTACTGATCAAACGGTATTCCTT
AGAGACACAAGTACTACTTTTAACATAAATGGTTGGTACTTTGCCTTATTC
TTTTGTATTTATATTATTATTTTCTATCGAAAAATCAAAATACGACAAATAT
TTTGAAACGACGAATAATTCATTCATTCATTTATCTCTTGTATGTGTAGGGT
ACACCATACCCAAAGGGTGGAAGTTTTTTGCAGTTGTATGGAATATTCATA
TGAATCCTGATGTTTATGTTCAGCCTAAGGAATTTAATCCTTCAAGATGGG
ATGTAAGTGATATGCATCTTAATTAATTGTGTTTAAGAAAAAAAAATTCAC
TTTATTTTTTTTATAATCAAGAAATTTTTGAGGGTTAATAATGTATGATTCA
AAATATGTTACGTAATAAGTTGTGTTTGCCTTTTACCACTTTGAATATTAGG
TATTTTATTTGCAGTAGGGTTTAAACTCATGACGAGAATTTATTTATACATA
AGAATATTAGTAAAATTAGCTCATAAAAATATGATTTGTTCAATGTCTTAT
TTATTAATTCAATTAATTTTGACTCATCTAATTTAATTCATCTAAAAAATAG
TGCTAGGATAATAATAATAATAATAATAAAATCGGAAATGTGCTCTAAAC
TAGAACCACGCCCTCCACTAGGAAGAAAAAAAATTGATTAACAATCTCTT
AACATTCTTCAAACCTAATTTTGAACCTCGGTATGTTCTCCTATTTTGAGTT
ATAGCCTCGATGAGTTAAAGACGAATCATGTCATATCTAGCTAATCAATTC
TTTTCAAATCTTCCTCGATCTATCTCTACCTCTCCTTAAACATCGTTGTCAA
TCTTTTGTACCTTAGGAGTAAAAGATAGGATATTAGTTGCTTTACCGTTCTC
AATGGTACATAGCTAAAAAGTGTATTATCATCAGGACAATGCATGTGTTCC
TCCTTTTGAGATATCTGAACTATCTCAATTTAACTTTTTACGTTACGAAAAT
CATTCCCACATTACTTATTACTTAATGATCAAGTGAAATTTTGACAGGATA
TTGAAACTAAGCCAGGCATTTTTCTTCCATTTTCAATGGGCCCCAAATCAT
GCCCAGGATCCAATCTGGCCAAGCTTCAAATTTCAGTAATTCTTCATTATT
ATCTTCTTCACTACAGGTAATTAAATAGCTTCATACTTATAATATGAAAAA
ATTAACTAATTATATTTTGAATATTTTATTTTTATTTTCTTACCAAATATTTT
CAAAGGGACTAAAATATATGAATTTATTGTGAAATTTATACCAAAAAATA
AGTATTTTGACCCTTATACTCGTGATTTTATCGTATGAATTGAGATGGATG
GAATATTTAAAATAAAAAAGGAAATATAAATTATTGATTAAATCATATTTT
AATTTAATTAAACGATATATTTAATATTCATGCAGGGTTGAGCAAATTAAT
CCAGAGGCTAGATGTTATCCTCCTGAAAATTGTCTTGTGAAATTCAAGAAG
CTCTCAATCTCTAGTGATGGTAACTAATTTTAATCATTGTGTTACAAATAA
ATAATTCATGTTGTAATCTTGAAAACAATAAGTGTATTGAATAATTATTAA
ATTGGATATTTTTGTAGAACGTTTAATCCAGTTTCAAATCTAGTTGGATAC
AGAGTTACTTTTATCAGAAAATGTTACATCTTCTGATTTTATTTTTAAATGA
AAAATATTTTGTATTTCCTTAATAGAAAAATCTTAAATTCATCAGTGTTTAA
ATGCATAGTCAAACAAACCCTAAATTATATGAATTTGATACTAACAAGTTT
GTTTTATCTTTCCCACCACCTGTATCGAATCGGATCGAGGATGATAAAAGA
AAATTATAAACTATTTTATTAAAAATATATGAAATATTTTGGACGGGAGGG
AGCACAGAACACCACTTCATTAAATTTTGTGACATTTAATTAATGTCGGAC
TTGAATCTTGATAATCACATCATATTTTATATTTCGAATTTTATATTAATTG
TTTATTTTTTTAAAAAAAATTATTTGTTGCATAGTGTATTCCACTTGATCTT
TCAATGTAAAAGAGGCTCTTTATTAAATTTGATAGCAGAGTTATATTA,
or a homolog or an ortholog thereof, having at least 70-100% homology
thereto.

In some embodiments, the genetically modified plant or plant part comprises a comprises a mutation in Exon 1, Exon 3, or both, of a gene encoding the GAME4 enzyme. In some embodiments, the genetically modified plant or plant part comprises a comprises a mutation in Exon 1 and a mutation in Exon 3 of a gene encoding the GMAE 4 enzyme (SEQ ID NO:2).

In some embodiments, the mutation is in nucleotide at position 155 of Exon 1 of the gene encoding GAME4 enzyme. In some embodiments, the mutation is in nucleotide at position 253 of Exon 1 of the gene encoding GAME4 enzyme. In some embodiments, the mutations are in nucleotides at positions 155 and 253 of Exon 1 of the gene encoding GAME4 enzyme.

In some embodiments, the mutation in nucleotide at position 155, 253, or both, of Exon 1 of the gene encoding GAME4 enzyme in a potato or in an equivalent position in an ortholog GMAE4 encoding gene in a plant belonging to the Solanum genus.

In some embodiments, the plant is a tomato plant or an eggplant plant, and the mutation is in a nucleotide located in Exon 1 of the GAME4 enzyme encoding gene in a position equivalent or homologous to position 155, 253, or both, of Exon 1 in the potato GAME4 enzyme encoding gene (SEQ ID NO:2).

The sequence of GAME4 enzyme encoding gene is known and would be apparent to one of ordinary skill in the art, such as from PubMed. Further, sequence alignment of GAME4 enzyme encoding genes can easily be employed by a skilled artisan so as to determine a nucleotide being at a position equivalent or homologous to position 155, 253, or both, of Exon 1 of the potato GAME4 enzyme encoding gene.

In some embodiments, the genetically modified plant or plant part comprises a mutation in Exon 1 of a gene encoding the DOX16 enzyme, and at least one mutation in Exon 1 of a gene encoding the GAME4 enzyme. In some embodiments, the genetically modified plant comprises a mutation in nucleotide at position 79 of Exon 1 of the gene encoding DOX16 enzyme, and a mutation at position 155, 253, or both, of Exon 1 of the gene encoding GAME4 enzyme. In some embodiments, the mutation is in nucleotide at position 79 of Exon 1 of the gene encoding DOX16 enzyme in a potato or in an equivalent position in an ortholog DOX16 encoding gene in a plant belonging to the Solanum genus, and in nucleotide at position 155, 253, or both, of Exon 1 of the gene encoding GAME4 enzyme in a potato or in an equivalent position in an ortholog DOX16 encoding gene in a plant belonging to the Solanum genus.

In some embodiments, the plant is a tomato plant or an eggplant plant, and the mutation is in a nucleotide located in Exon 1 of the DOX16 enzyme encoding gene in a position equivalent or homologous to position 79 of Exon 1 in the potato DOX16 enzyme encoding gene (SEQ ID NO:1), and in a nucleotide located in Exon 1 of the GAME4 enzyme encoding gene in a position equivalent or homologous to position 155, 253, or both, of Exon 1 in the potato GMAE4 enzyme encoding gene (SEQ ID NO:2).

In some embodiments, the mutation comprises a deletion or insertion.

In some embodiments, the mutation is a deletion. In some embodiments, the mutation is an insertion.

In some embodiments, the insertion or deletion is of at least 1 nucleotide, at least 2 nucleotides, at least 3 nucleotides, at least 5 nucleotides, at least 7 nucleotides, at least 9 nucleotides, at least 11 nucleotides, at least 13 nucleotides, or at least 15 nucleotides, or any value and range therebetween. Each possibility represents a separate embodiment of the invention. In some embodiments, the insertion or deletion is of 1-5 nucleotide, 1-7 nucleotides, 1-9 nucleotides, 3-12 nucleotides, 5-9 nucleotides, 2-11 nucleotides, or 1-15 nucleotides. Each possibility represents a separate embodiment of the invention.

In some embodiments, the mutation is introduced into an exon of a gene as disclosed herein using a programmable engineered nuclease (PEN).

In some embodiments, a PEN comprises a clustered regularly interspaced short palindromic repeat (CRISPR) type II system a gene-editing method.

Any mutation(s) can be inserted into an endogenous polynucleotide encoding the at least one enzyme, including deletions, insertions, site specific mutations including nucleotide substitution and the like, as long as the mutation(s) result in down-regulation of the gene expression or in the production of less-functional or non-functional protein.

Any method for mutagenesis as is known in the art can be used according to the teachings of the present invention including chemical mutagenesis, radio-mutagenesis and site directed mutagenesis, for example using genome editing techniques. According to some embodiments, the plants of the invention are produced by inserting a mutation using the CRISPR/Cas system, a CRISPR/Cas homologous and CRISPR/Cas modified systems.

The CRISPR/Cas system for genome editing contains two distinct components: a gRNA (guide RNA) and an endonuclease e.g., Cas9.

The gRNA is typically a 20-nucleotide sequence encoding a combination of the target homologous sequence (crRNA) and the endogenous bacterial RNA that links the crRNA to the Cas9 nuclease (tracrRNA) in a single chimeric transcript. The gRNA/Cas9 complex is recruited to the target sequence by the base-pairing between the gRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break. Comparable with other genome editing nucleases, Zinc-finger nucleases (ZFNs) and transcription activator-like effector nucleases (TALENs), the double-stranded brakes produced by CRISPR/Cas can undergo homologous recombination or nonhomologous end-joining (NHEJ).

The Cas9 nuclease has two functional domains: RuvC and HNH, each cutting a different DNA strand. When both of these domains are active, the Cas9 causes double strand breaks in the genomic DNA.

A significant advantage of CRISPR/Cas is that the high efficiency of this system coupled with the ability to easily create synthetic gRNAs enables multiple genes to be targeted simultaneously. In addition, the majority of cells carrying the mutation present bi-allelic mutations in the targeted genes.

However, apparent flexibility in the base-pairing interactions between the gRNA sequence and the genomic DNA target sequence allows imperfect matches to the target sequence to be cut by Cas9.

Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. A single-strand break, or nick, is normally quickly repaired through the HDR pathway, using the intact complementary DNA strand as the template. However, two proximal, opposite strand nicks introduced by a Cas9 nickase are treated as a double-strand break, in what is often referred to as a ‘double nick’ CRISPR system. A double-nick can be repaired by either NHEJ or homology directed repair (HDR) depending on the desired effect on the gene target. Thus, if specificity and reduced off-target effects are crucial, using the Cas9 nickase to create a double-nick by designing two gRNAs with target sequences in close proximity and on opposite strands of the genomic DNA would decrease off-target effect as either gRNA alone will result in nicks that will not change the genomic DNA.

Modified versions of the Cas9 enzyme containing two inactive catalytic domains (dead Cas9, or dCas9) have no nuclease activity while still able to bind to DNA based on gRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

There are number of publicly available tools to help choose and/or design target sequences as well as lists of bioinformatically determined unique gRNAs for different genes in different species such as the Feng Zhang lab's Target Finder, the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

In order to use the CRISPR system, both gRNA and Cas9 should be expressed in a target cell. The insertion vector can contain both cassettes on a single plasmid or the cassettes are expressed from two separate plasmids.

None limiting example for gene editing technology or methodology comprises the MEMOGENE™ technology.

In some embodiments, the plant or plant part is devoid of heterologous polynucleotides. In some embodiments, the plant or plant part is devoid of exogenous polynucleotides. As used herein, the terms heterologous or exogenous encompass nucleic acid sequence(s) non-naturally occurring or being present in a plant.

Genetically modified plants or plant derived therefrom, including, but not limited to potato haulm as disclosed herein, being obtained by gene editing typically do not contain exogenous polynucleotides within their genome, and are therefore characterized as non-transgenic plant. The present invention thus provides non-transgenic haulm, which is better acceptable as a feed for livestock animals.

According to some embodiments, expression of the endogenous gene is affected at the genomic and/or the transcript level using a variety of molecules that interfere with transcription and/or translation (e.g., antisense, siRNA, Ribozyme, or DNAzyme) of the gene.

According to some embodiments, the present invention is directed to the use of a feed composition comprising potato haulm essentially devoid of SGAs and having IVDMD essentially equivalent to the IVDMD of a wild type (WT) potato haulm comprising SGAs for feeding ruminant arm animals.

In some embodiments, a control plant comprises a wild type (WT) plant belonging to the genus Solanum. In some embodiments, a control plant comprises a genetic background line or strain of the genetically modified plant disclosed herein.

In some embodiments, a control plant comprises a genetically modified, mutated, transgenic, or any combination thereof, of a plant belonging to the Solanum genus, as long as it: (i) does not include a mutation as disclosed herein, (ii) does not have an activity equivalent to the genetically modified plant disclosed herein, e.g., low SGA content and/or IVDMD greater than or essentially equivalent, or (iii) both.

In some embodiments, the edible composition disclosed herein is for use in feeding of an animal subject. In some embodiments, the edible composition disclosed herein is suitable for use in feeding of an animal subject. In some embodiments, any one of: low (or being devoid of) SGA, IVDMD greater than or essentially equivalent to the IVDMD of a control plant, a protein content of 35-50% weight per weight of dry matter of the plant or plant part, potassium in an amount of 3-10% weight per weight of dry matter of the plant or plant part, at least one essential amino acid being selected from: Histidine, Isoleucine, Leucine, Lysine, Phenylalanine, Threonine, Valine, or any combination thereof, in an amount being at least 10% by weight greater that in a control plant, or any combination thereof, renders the edible composition disclosed herein suitable for use in feeding an animal subject.

In some embodiments, the animal subject comprises a mammal subject. In some embodiments, the animal subject comprises poultry species or subjects.

In some embodiments, the mammal subject comprises a farmed mammal. In some embodiments, the mammal subject is a livestock animal.

In some embodiments, the livestock animal is or comprises a ruminant farm animal.

Types of farmed mammals, including, but not limited to ruminant farm animals are common and would be apparent to one of ordinary skill in the art. None-limiting examples of such mammals include, but are not limited to: cattle, sheep, horse, swine, camel, and the like.

In some embodiments, the animal subject is selected from: human, livestock, horse, swine, camel, poultry, or any combination thereof.

In some embodiments, livestock comprises cattle, sheep, or both.

In some embodiments, a mammal subject comprises a human subject. In some embodiments, when referring to a human subject, the edible composition disclosed herein is or comprises food or an ingredient thereof.

In some embodiments, when referring to a farmed mammal, such as a livestock animal, the edible composition disclosed herein is or comprises feed or an ingredient thereof.

According to some embodiments, there is provided a genetically modified plant or a part thereof, the plant belonging to the genus Solanum characterized by being essentially devoid of SGAs and having IVDMD greater than or essentially equivalent to the IVDMD of a control plant.

In some embodiments, the genetically modified plant comprises at least one enzyme being selected from: 16DOX, GAME4, and both, and is characterized by having reduced expression and/or activity compared to the expression and/or activity of the at least one enzyme in a control plant.

In some embodiments, the plant part is selected from: haulm, tuber, seed, fruit, leaf, flower, stem, root, pollen, foliage, or any combination thereof.

In some embodiments, the plant part comprises a cell culture, a primary cell culture, a cell line, a stable cell line, an immortalized cell line, a callus, or any combination thereof, being obtained or derived from a genetically modified plant as disclosed herein.

According to some embodiments, there is provided a method for producing a plant or plant part being essentially devoid of SGAs and having an IVDMD being greater than or essentially equivalent to the IVDMD of a control plant.

In some embodiments, the method comprises mutating at least one gene encoding at least one enzyme being selected from: 16DOX, GAME4, and both, such that the at least one enzyme is characterized by having reduced expression and/or activity compared to the expression and/or activity of the at least one enzyme in a control plant or plant part.

In some embodiments, mutating is by gene editing, such as by CRISPR-Cas9 system as disclosed herein.

In some embodiments, the method comprises contacting a plant cell, with a Cas9 protein or a first exogenous polynucleotide encoding same and at least one guide RNA (gRNA) or a second exogenous polynucleotide encoding same.

In some embodiments, the gRNA is selected from: CCCAGTGCATGAAAGACCAT (SEQ ID NO:6), TATATGGGTTTGCCATATTT (SEQ ID NO:9), TTTGGCTTTCTCCAAGAAAT (SEQ ID NO:10), or any combination thereof.

In some embodiments, the method comprises contacting a DOX16 enzyme encoding gene, a GAME4 enzyme encoding gene, or both, in a plant cell, with a Cas9 protein or a first exogenous polynucleotide encoding same and at least one guide RNA (gRNA) or a second exogenous polynucleotide encoding same. In some embodiments, the first and second exogenous polynucleotides are a single exogenous polynucleotide.

In some embodiments, the genetically modified plant or part thereof as disclosed herein is produced with SEQ ID NO: 6. In some embodiments, the genetically modified plant or part thereof as disclosed herein is produced with SEQ ID NO: 9. In some embodiments, the genetically modified plant or part thereof as disclosed herein is produced with SEQ ID NO: 10. In some embodiments, the genetically modified plant or part thereof as disclosed herein is produced with SEQ ID NOs: 6 and 9. In some embodiments, the genetically modified plant or part thereof as disclosed herein is produced with SEQ ID NOs: 6 and 10. In some embodiments, the genetically modified plant or part thereof as disclosed herein is produced with SEQ ID NOs: 9 and 10. In some embodiments, the genetically modified plant or part thereof as disclosed herein is produced with SEQ ID NOs: 6, 9 and 10.

### Definitions

As used herein, the terms “haulm” and “potato haulm” are used herein interchangeably and refer to the aerial parts of a potato plant, including stems and leaves.

As used herein, the terms “feed” or “food” refer to a nutrition product, a composition, or an ingredient thereof, particularly to nutrition product/composition suitable for feeding or nourishing a subject, including a mammal subject, including, but not limited to a human subject, a farmed animal, a ruminant farm animals, or the like.

The term “expression”, as used herein, refers to the production of a functional end-product e.g., an mRNA or a protein.

As used herein, the expression and/or activity with regard to at least one enzyme involved in the biosynthesis of SGAs is “reduced”, “inhibited”, “down regulated” or “knocked out” or “knocked down” if the level of the polynucleotide, the encoded protein and/or the protein measured activity is reduced by at least 60%, at least 65%, at least 70%, at least 75%, at least 80%, at least 85%, at least 90%, at least 91%, at least 92%, at least 93%, at least 94%, %, at least 95%, at least 96% at least 97%, at least 98%, at least 99%, or more compared to its level in a control plant. According to some embodiments, the term “reduced expression and/or activity” refers to 100% inhibition or “full knockout” of the gene. According to certain exemplary embodiments, the reduced expression and/or activity of the at least one enzyme results in SGA content that is reduced by at least 60%, at least 65%, at least 70%, at least 75%, at least 80%, at least 85%, at least 90%, at least 91%, at least 92%, at least 93%, at least 94%, %, at least 95%, at least 96% at least 97%, at least 98%, at least 99%, or more compared to its level in a control plant.

The term “genetically modified” with regard to a potato plant or a haulm derived therefrom refers to a potato plant or haulm comprising at least one cell genetically modified by man. According to certain exemplary embodiments of the invention, the genetic modification includes modification of an endogenous gene(s), for example by introducing mutation(s) deletions, insertions, transposable element(s) and the like into an endogenous polynucleotide or gene of interest. Accordingly, the potato plants of the invention and the haulm derived therefrom are genetically modified but not transgenic, i.e., do no comprise heterologous polynucleotides.

As used herein, “sequence identity” or “identity” in the context of two polypeptide or nucleic acid sequences includes reference to the residues in the two sequences which are the same when aligned. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are considered to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Henikoff S and Henikoff J G. (Amino acid substitution matrices from protein blocks. Proc. Natl. Acad. Sci. U.S.A. 89 (22), 10915-9, 1992).

Identity (e.g., percent homology) can be determined using any homology comparison software, including for example, the BlastN, BlastX or Blastp software of the National Center of Biotechnology Information (NCBI) such as by using default parameters.

According to some embodiments of the invention, the identity is a global identity, i.e., an identity over the entire amino acid or nucleic acid sequences of the invention and not over portions thereof.

The term “gene” refers to a nucleic acid (e.g., DNA or RNA) sequence that comprises coding sequences necessary for the production of RNA or a polypeptide. A polypeptide can be encoded by a full-length coding sequence or by any part thereof. The term “parts thereof” when used in reference to a gene refers to fragments of that gene. The fragments may range in size from a few nucleotides to the entire gene sequence minus one nucleotide. Thus, “a nucleic acid sequence comprising at least a part of a gene” may comprise fragments of the gene or the entire gene.

The term “gene” also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences.

The terms “polynucleotide”, “polynucleotide sequence”, “nucleic acid sequence”, and “isolated polynucleotide” are used interchangeably herein. These terms encompass nucleotide sequences and the like. A polynucleotide may be a polymer of RNA or DNA or hybrid thereof, that is single- or double-stranded, linear or branched, and that optionally contains synthetic, non-natural or altered nucleotide bases. The terms also encompass RNA/DNA hybrids.

According to some embodiments, the present invention provides a livestock feed comprising potato haulm, wherein the haulm is essentially devoid of steroidal glycoalkaloids (SGAs), and wherein the in vitro dry matter digestibility (IVDMD) of the haulm is essentially equivalent to the IVDMD of a wild type (WT) potato haulm comprising SGAs.

SGAs consist of two structural components: the aglycone unit composed of nitrogen containing C27 steroid derived from cholesterol and oligosaccharide attached to the hydroxy group at C-3. Based on the skeletal structure of the aglycone, SGAs can be divided into two general classes, solanidane or spirosolane. Minor structural variations of these two ring types such as C-5 saturation/unsaturation or isomerization at C-22, in combination with various sugar moieties, generate the enormous structural diversity of SGAs. In addition, their chemical structures reflect their biological activities, for example, toxicity to animals, anti-cancer properties, and anti-microbial activities. Most representatives of solanidane glycoalkaloids are toxins, α-solanine and α-chaconine, that comprise upward of 90% of the total SGAs in cultivated potatoes. Other SGAs include tomatine.

SGA biosynthesis can be divided into two main parts: aglycone formation and glycosylation. Recent research in potato and tomato identified several SGA biosynthetic genes involved in aglycone formation. Three cytochrome P450 monooxygenases (CYPs) named as PGA2 (GAME7), PGA1 (GAME8), PGA3 (GAME4) have been found to be involved in hydroxylation of cholesterol at C-22 and C-26 and oxygenation at C-26, respectively. A 2-oxoglutarate-dependent dioxygenase (DOX) named as 16DOX (GAME11), and an aminotransferase were reported to be required for the C-16α-hydroxylation and C-26 amination during SGA biosynthesis. These enzymes and functions are common to potato and tomato, suggesting that they are involved in the biosynthetic steps common to solanidanes and spirosolanes. In addition, several uridine diphosphate-dependent glycosyltransferases (UGTs) involved in the glycosylation steps of SGA biosynthesis have been identified in potato and tomato (Akiyama, R et al., 2021. Nat Commun 12, 1300. doi.org/10.1038/s41467-021-21546-0).

According to certain embodiment, the haulm essentially devoid of steroidal glycoalkaloids (SDAs) is genetically modified to have reduced expression and/or activity of at least one enzyme involved in the biosynthesis of SGAs in potato haulm cells compared to the expression and/or activity of the at least one enzyme in a corresponding unmodified potato haulm.

Any method as is known in the art for reducing the expression and/or activity of the at least one enzyme involved in the SGA biosynthesis pathway can be used with the teachings of the invention.

According to certain exemplary embodiments, reducing the expression and/or activity in obtained by reducing the expression of the endogenous gene or mRNA encoding the enzyme. According to some embodiments, reducing the expression of the endogenous gene or mRNA is obtained by inserting at least one mutation in the endogenous gene/mRNA.

According to certain embodiments, the edible composition consists of the haulm of a plant belonging or classified to the genus Solanum.

As used herein, the term “about” when combined with a value refers to plus and minus 10% of the reference value. For example, a length of about 1000 nanometers (nm) refers to a length of 1,000 nm±100 nm.

It is noted that as used herein and in the appended claims, the singular forms “a”, “an”, and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely”, “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

According to certain additional and/or alternative embodiments, the edible composition further comprises additional nutritional components. The additional nutritional components can be natural (e.g., plant material of other plant species, fresh or processed) or synthetic (e.g., vitamins, hormones, antibiotic etc.).

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization-A Laboratory Course Manual” CSHL Press (1996); all of which are incorporated by reference. Other general references are provided throughout this document.

### Materials and Methods

### SpCas9 Guides Design and Construct

In order to knock out the activity of Solanum tuberosum (crop cultivars Desiree and Pfifer) genes DOX16 (SEQ ID NO:1), GAME4 (SEQ ID NO:2) and SSR2 (SEQ ID NO: 3) by SpCas9, three sgRNAs were designed for each gene. Target at the 5′ of the genes were searched for. The inventors looked for high scoring Cas9 spacers comparing tables from three different web sites for Cas9 digestion design. (crispor.tefor.net/, chopchop.cbu.uib.no/, cbi.hzau.edu.cn/cgi-bin/CRISPR2/SCORE, and rgenome.net/cas-designer/).

Cas9 sites in which a mutation at the DNA will also disable a restriction enzyme site were searched for. This enabled the inventors to evaluate the activity of the enzyme at each site, to: (i) screen mutated regenerated plants, (ii) know if mutations occur in all alleles of the target gene or not, and (iii) isolate and sequence the mutation in order to verify if the gene was knocked.

In this procedure, a DNA was extracted from Cas9 treated S. tuberosum tissue and the Cas9 target site was amplified by PCR. Then the amplicon was digested by the restriction enzyme that has a unique site at the Cas9 digestion site. Non-digested amplicon indicates mutation and it can be isolated and sequenced if needed. The selection enzymes for each spacer are listed in Table 1 and the primers used for the PCR are presented at Table 2.

The 20-nucleotides variable part of the sgRNA (spacer) was ordered as two complimentary oligomers. The single strand oligonucleotides annealed to form the double strand oligonucleotide. Each of the sense oligomers starts with four nucleotides that complement the sequence of the digested promoter upstream to the spacer. Each of the antisense oligomers starts (at 5′) with AAAC four nucleotide at the beginning of the sgRNA scaffold and hybridized to them, located downstream to the spacer. Therefore, the spacer was used as an adaptor to connect between the promoter and the sgRNA scaffold.

TABLE 1
List of all Cas9 sgRNAs and spacers sequences

| | | | SEQ | |
|---|---|---|---|---|
| | | | ID | Selection |
| Target | Name | Sequence | NO: | Enzyme |
| Mutated | QQR | GTAATTCTTCCCCTCAGGAG | 4 | Bsu36I |
| GUS | | | | |
| DOX16 | DOX16- | CCGATGGTCTTTCATGCACT | 5 | RseI |
| | Ex1-68 | | | |
| DOX16 | DOX16- | CCCAGTGCATGAAAGACCAT | 6 | BccI |
| | Ex1-79 | | | |
| DOX16 | DOX16- | TGCTTCATCCATTAGATCTA | 7 | BglII |
| | Ex2-25 | | | |
| GAME4 | GAME4- | TATTAATGAGAATTAATGGT | 8 | AjuI |
| | Ex1-84 | | | |
| GAME4 | GAME4- | TATATGGGTTTGCCATATTT | 9 | Van91I |
| | Ex1-155 | | | |
| GAME4 | GAME4- | TTTGGCTTTCTCCAAGAAAT | 10 | Van91I |
| | Ex3-253 | | | |
| SSR2 | SSR2-52 | CCCTAGGAGGAAGATCCAGT | 11 | BsrI |
| SSR2 | SSR2-631 | ACGCTATTCCGTGGTCTCAA | 12 | BsaI |

TABLE 2
PCR Primers (for Cas9 target site)

| | | SEQ |
|---|---|---|
| | | ID |
| Primer Name | Sequence | NO: |
| QQR Forward | CTATCCTTCGCAAGACCCTTCC | 13 |
| QQR Reverse | GTCTGCCAGTTCAGTTCGTTGTTC | 14 |
| DOX16 Ex1-68 or 79 | CATTTAGAAGATTTCTTTCTTTCCC | 15 |
| Forward | | |
| DOX16 Ex1-68 or 79 | GGGTACTCCATGATTGATTATC | 16 |
| Reverse | | |
| DOX16 Ex2-25 | GAAGAATATGGGTTTTTTCAGG | 17 |
| Forward | | |
| DOX16 Ex2-25 | TCTCCAGTAACGATGCTCCTC | 18 |
| Reverse | | |
| GAM4 Ex1-84 or 155 | TTGAAAGGAAAAAGGTTAATATGGT | 19 |
| Forward | | |
| GAME4 Ex1-84 or | GCTTTGTCACCATAATTGTTGG | 20 |
| 155 Reverse | | |
| GAM4 Ex3-253 | CGAAAAGTACTAGGAGGGATATTTC | 21 |
| Forward | | |
| GAME4 Ex3-253 | CTTTACTATTTCCTCTTGTTCCTCC | 22 |
| Reverse | | |
| SSR2 -52 Forward | CAAATGCTTGTTATATCATGTTGCTAA | 23 |
| SSR2 -52 Reverse | ACCATCCTTTTCTGCATTCC | 24 |
| SSR2 -631 Forward | CCAAAGTTGAGCCTCTTGTCA | 25 |
| SSR2 -631 Reverse | CATCTTTAGGTGCAAAAGAATCC | 26 |

### Plasmids Construction

The plasmid with the human (h) codon usage of SpCas9 for stable transformation (#4064, SEQ ID NO:27) was cloned with an Arabidopsis thaliana (At) Ubiqutin promoter and CaMV 35S terminator. This Cas9 cassette was cloned into the binary plasmid pCGN1559 that contain also NptII as selection gene under the CaMV 35S promoter.

Plasmid with mutated GUS that serve as target to the activity assay, as described (Tovkach A et al., 2009. The Plant J. 57, 747-757).

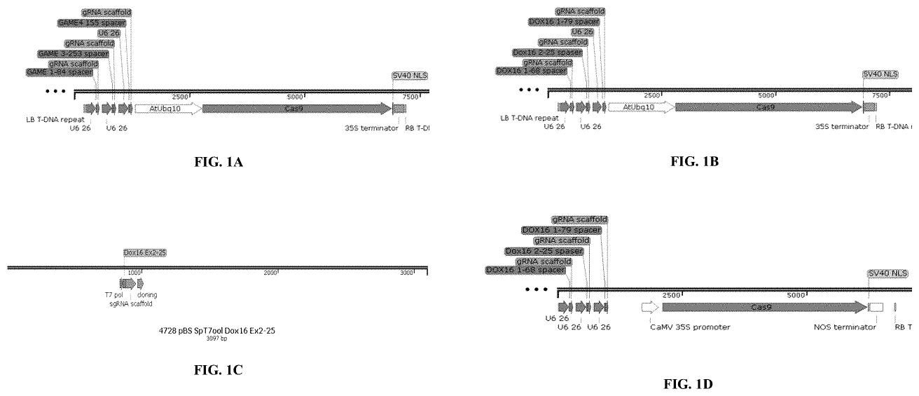

The plasmids with three U6-sgRNA and Cas9 for transient activity in protoplasts do not contain selection gene. The inventors cloned the AtUbi promoter or CaMV 35S promoter to induce the transcription of the hSpCas9. The binary vector is based on pZP-2000 RCS adjusted to clone by type-II restriction enzymes. This enables to add the three U6-sgRNAs to the plasmid with the Cas9. A schematic description of the plasmid is presented in FIG. 1.

### Protoplast Extraction and Transfection

Protoplast isolation, transfection and plant regeneration were based essentially on a work previously described (Nicolia A et al. 2015, J Biotechnol 204:17-24). Briefly, Young healthy leaves from in vitro propagated potato plants shoot were excised for protoplast isolation. Leaves were cut vertically and put into digestion solution composed of 1% cellulose (Duchefa) and 0.2% macerozyme (Duchefa) in BNE9 solution for 15 h. Next day protoplasts were filtered and washed 4 times using W5 washing solution and counted using hemocytometer.

The purified protoplasts were transfected in either of two ways: (1) Protoplast transfection using DNA plasmids (one or more at the same transfection). Most of the mutated plants were obtained by this method (Table 3, plants Nos. 1-62); or (2) Protoplast transfections using Cas9 as protein and RNA guide (DNA free system). This transfection led to 1 mutated plant (Plant No. 63)

Each transfection included 1×10⁶ protoplasts mixed with 30% PEG 4000 and 10 μg of each DNA plasmid used or alternatively 10 μg Cas9 protein and 10 μg of RNA guide. The transfection reaction was performed at room temperature for 5 min. After transfection, the protoplasts were washed 4 times and embedded in 0.5 ml drops of culture medium-alginate solution. Transfected protoplasts were incubated at 24° C. in darkness for one-week, light intensity was gradually increased to 600 lux until calli were formed. Four to six (4-6) weeks post transfection, calli were released from alginate media and incubated in liquid media for several weeks resulting in further callus development and shoot induction. When enlarged green calli were formed, they were moved into solid media and 2,000 lux light intensity, for further development of shoots. From the three potato varieties examined (Desiree, Pfifer, and Nicola), regeneration was most successful for Desiree and somewhat successful for Pfifer. Accordingly, the variety Desiree was mostly used in the experiments.

### Preparation of Cas9 Transgenic Potato Plants

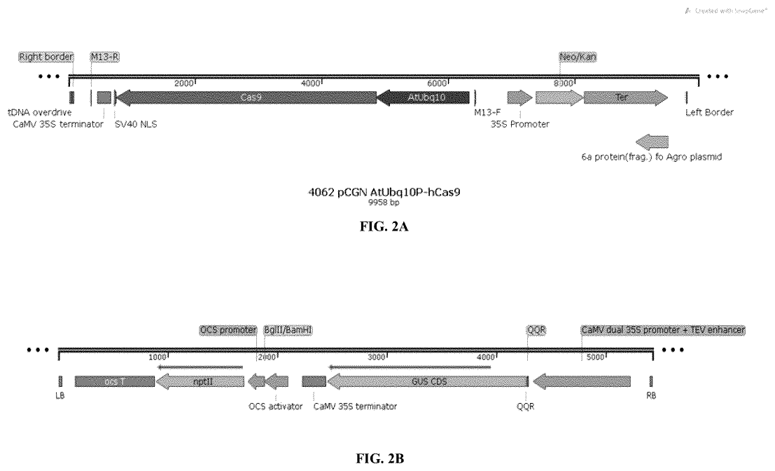

A binary construct that contained the UbiP: hCas9 cassette and a kanamycin selection marker (plasmid #4064, FIG. 2A) was mobilized to Agl-0 Agrobacterium tumefaciens by electroporation. Single positive clone was PCR verified and used to initiate a culture for potato transformation according to the protocol described by Craze et al., with some modifications (Craze et al., 2018. Current protocols in Plant Biology 3, 33-41). In short, leaves were isolated from aseptically grown potato plants. The petioles and leaf edges were all removed such that the leaves were squared off. The leaves were then divided into 5×5 mm squares to generate leaf disk explants. The Agrobacterium culture was grown overnight at 28° C. with appropriate selection. On the day of co-cultivation, the OD600 of Agrobacterium culture was adjusted to 0.25 using 10 mM MgCl₂ Solution. The culture was supplemented with acetosyringone (A.S.) 20 mg/l=100 μM final. The leaf disk explants were placed into a sterile petri dish along with 10 ml of the Agrobacterium culture. The inoculation was done for 20-minute incubation time. The Agrobacterium solution was removed and the explants were blotted on sterile filters to remove any remaining liquid. The explants were plated onto Co-cultivation Medium (PCM) with A.S. 20 mg/l. For the co-cultivation, explants should be placed on their abaxial side down onto the medium. The explants were cultured in the dark at 25° C. for 2 days. Then, the explants were gathered and washed with sterile deionized water containing carbenicillin 300 mg/l and timentin 300 mg/l and Plant Preservation Medium (PPM) 0.2%, to remove the Agrobacterium from their surfaces. The washed leaf disks were then plated abaxially onto Regeneration Medium as described by Molla et al., (2011. The 2011 International Conference on Environment and Industrial Innovation IPCBEE Vol. 12 IACSIT Press, Singapore) and grown in the light (16 h light/8 h dark) at 25° C. Leaf explants were placed 16-20 per plate. The Regeneration medium contained carbenicillin 300 mg/l and timentin 300 mg/l and PPM 0.2%. For selecting transgenic plants, the Regeneration Medium also contained kanamycin 100 mg/l. Shoots that have grown out for one or more centimeters in length were excised and placed into plastic deli cups containing 80 ml of MS medium with 8 μM silver thiosulfate (STS), carbenicillin 300 mg/l, timentin 300 mg/l and PPM 0.2%. No selection is performed at this stage. The regenerated plants were moved to rooting media, and rooting occurred as early as several days and up to 2 weeks of culture. Once hardened, the plants were sampled for genomic DNA extraction using CTAB plant DNA extraction method. PCR analysis was used to verify the presence of the Cas9 transgene in the sampled plantlets. Overall, 8 Desiree plants and 8 Pfifer plants were PCR positive for Cas9, and were further in vitro propagated.

### Examining of Cas9 Activity in Cas9 Transgenic Potato Plants

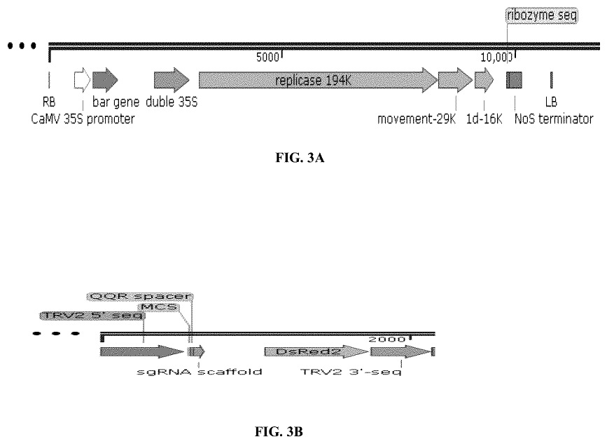

Examining of Cas9 in-vivo activity was performed by applying a transient assay for mGUS activation. Briefly, a binary pCGN-mGUS plasmid (plasmid #1453, SEQ ID NO: 28, FIG. 2B) carries a mutated GUS that is transiently expressed in the tissue following inoculation for at least 72 hours. The Tobacco rattle virus TRV1-TRV2 components (Maps #1713, SEQ ID NO:29 and #3354, SEQ ID NO:30, FIGS. 3A-3B, respectively) carry a guide RNA that targets the mGUS in its target sequence that is termed “QQR”. Once precisely targeted, mGUS can be changed to GUS and the presence of active GUS can be detected with proper staining of the inoculated tissue. If a specific plant has an active Cas9 protein and was infected successfully with this transient system, appearance of blue GUS staining dots is expected.

Small leaf pieces from the Cas9 positive Desiree and Pfifer plants were inoculated by a combination of the following 3 Agrobacterium lines, each carries a component of the transient assay for mGUS activation: pTRV1, pTRV2-sgQQR-DsRed, and pCGN-mGUS in a ratio of 1:1:2 and in final OD600=0.5 in 10 mM MgCl₂ solution. The leaves were co-cultivated with the Agrobacterium for 48 hrs in darkness, then washed and moved to Potato Regeneration Media (Molla et al., 2011, ibid+Cb300). DsRed analysis (used to detected TRV infected leaf pieces) was performed at 7 days post inoculation by a florescence binocular device (Nikon). DsRed positive leaf pieces were put into GUS solution, incubated overnight at 37° C., washed with 70% Ethanol and analyzed for GUS stain by light microscope. 5 out of 8 Desiree lines (named #6, #7, #9, #11 and #16) and 3 out of 8 Pfifer lines (named #12, #16 and #20) were found to have multiple GUS stains, thus providing a visual evidence of Cas9 activity. Lines Desiree #6, #7, #9 and Pfifer #16 and #20 had the most GUS staining thus indicating on their superior Cas9 activity. Those five specific lines were further propagated to be used for the target gene mutagenesis experiments.

sgRNA Design and Activity Test for Dox16 and SSR2 Target Genes

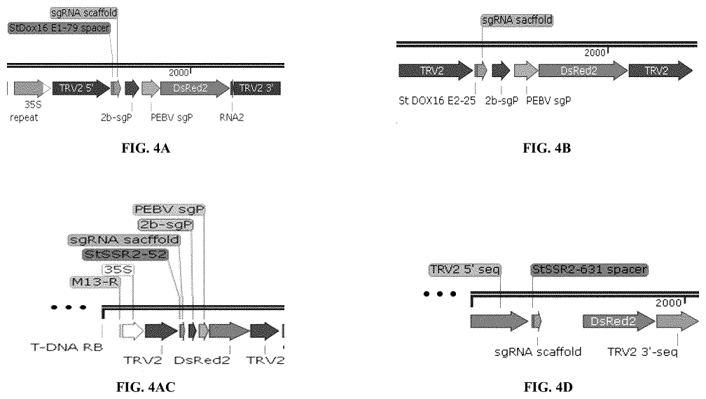

Two sgRNAs were designed for each of the 2 target genes. For DOX16 target gene, sg79-DOX16 (CCCAGTGCATGAAAGACCAT, SEQ ID NO:6) and sg25-DOX16 (TGCTTCATCCATTAGATCTA SEQ ID NO:7) targeting Exons 1 and 2 of the gene, respectively. For SSR2 target gene, sg52-SSR2 (CCCTAGGAGGAAGATCCAGT, SEQ ID NO:11) and sg631-SSR2 (ACGCTATTCCGTGGTCTCAA, SEQ ID NO: 12) both targeting exon 1 of the gene. All 4 guide RNAs were cloned to TRV2 vectors under sub-genomic promoter, creating the 4 viral constructs: TRV2-sg79-DOX16-DsRed (Map #8065, SEQ ID NO:31, FIG. 4A), TRV2-sg25-DOX16-DsRed (Map #4906, SEQ ID NO:32, FIG. 4B), TRV2-sg52-SSR2-DsRed (Map #8041, SEQ ID NO: 33, FIG. 4C), and TRV2-sg631-SSR2-DsRed (Map #8042, SEQ ID NO:34, FIG. 4D).

To identify the most active guide for each gene, all 4 gene specific guides were checked in a transient assay. Briefly, Agrobacterium lines carrying the TRV1 vector and one of the 4 TRV2 vectors were grown liquid LB media, containing 50 μg/mL Kanamycin and 20 μg/mL Acetosyringone for overnight (approx. 16 hours) at 28° C. with proper selection, then mixed in a ratio of 1:1 (TRV1:TRV2-sgRNA) to final OD600=0.5 in 10 mM MgCl₂ Solution. Cas9 Potato “Desiree” leaves were then prepared as described above in the “preparation of Cas9 plants” section. Approximately 25 leaf explants were incubated with each of the 4 Agrobacterium solution mixtures for 20 minutes, then co-cultivated for initial 48 hours in the darkness, and finally washed, moved back to light, and placed on Molla et al., 2011 (ibid) Regeneration Medium supplemented with Cb300. DsRed analysis (used to detected TRV infected leaf pieces) was performed at 7 days post inoculation by a florescence binocular device (Nikon). DsRed positive leaf pieces were sampled for genomic DNA extraction using CTAB method. Genomic DNA was used for PCR amplification of the 4 relevant amplicons of both genes. Each amplicon underwent the restriction site-loss method described hereinabove using a specific restriction enzyme for each of the guides. Uncut bands appeared in all 4 guides, demonstrating that there were all active, but there were differences in the intensity between them. Finally, by comparing the intensity of the uncut bands between each pair of gene specific guides, we decided to use TRV2-sg25-DOX16-DsRed to target the Potato DOX16 gene and TRV2-sg52-SSR2-DsRed to target the Potato SSR2 gene.

### Production DOX16 and SSR2 Mutants by Viral Inoculation of Cas9 Potato Plants

Cas9 transgenic potato plants from lines Desiree #6, #7, #9 and Pfifer #16 and #20 were chosen to be the source of plant material for the mutagenesis experiments based by their superior Cas9 activity as described above.

To produce mutations in the DOX16 target gene, we first prepared Agrobacterium mixture (1:1) of TRV1 and TRV2-sg25 DOX16 lines as described hereinabove, then inoculated the leaf pieces with this mixture at O.D.600-0.25 in 10 mM MgCl₂ Solution, co-cultivated in darkness for 48 hours and finally moved to Molla et al., 2011 Potato Regeneration medium supplemented with Cb300 for further regeneration. DsRed analysis (used to detected TRV infected leaf pieces) was performed at 7 days post inoculation by a florescence binocular device (Nikon). DsRed negative leaf pieces were eliminated and DsRed positive leaf pieces were left to further regenerate without any selection. Regenerated plantlets were moved to rooting media, and once fortified were sampled for genomic DNA extraction using a fast DNA extraction and PCR protocol as follows: Half of Eppendorf-cup size leaf disk was placed in a 0.2 ml PCR tube and 25 μl buffer A was added (Buffer A: 100 mM NaOH, 2 mM Tween 20%). The sample was placed in a PCR machine and heated for 10 min at 95° C., then cooled back to room temperature. Twenty-five (25) μl buffer B was then added and mixed (Buffer B: 100 mM Tris-HCl, 2 mM EDTA, pH=8). Two (2) μl of the treated sample was taken to a standard 20 μl PCR reaction without further cleaning.

To produce mutations in the SSR2 target gene, the inventors first prepared Agrobacterium mixture (1:1) of TRV1 and TRV2-sg52 SSR2 lines, then inoculated the leaf pieces with this mixture at O.D.600-0.25 in 10 mM MgCl₂ Solution, co-cultivated in darkness for 48 hours and finally moved to Molla et al., 2011 potato regeneration Medium supplemented with Cb300 for further regeneration. DsRed analysis (used to detected TRV infected leaf pieces) was performed 12 days post inoculation by a florescence binocular device (Nikon). DsRed negative leaf pieces were eliminated and DsRed positive leaf pieces were left to further regenerate without any selection. Regenerated plantlets were moved to rooting media, and once fortified were sampled for genomic DNA extraction using a fast DNA extraction and PCR protocol as described hereinabove.

### Measurement of α-Chaconine and α-Solanine Content

Steroidal compounds accumulated in the haulm (mainly leaves and stems) of the mutated and wild type potato were extracted from the fresh haulm with methanol.

Samples were analyzed on LC-MS system which consisted of Dionex Ultimate 3000 RS HPLC coupled to Q Exactive Plus hybrid FT mass spectrometer equipped with heated electrospray ionization source (Thermo Fisher Scientific Inc.).

The HPLC separations were carried out using Acclaim C18 column (2.1×150 mm, particle size 2.2 μm, Dionex) employing linear binary gradient of acetonitrile and water with 0.1% acetic acid.

The mass spectrometer was operated in positive ESI ionization mode, ion source parameters were as follows: spray voltage 3.5 kV, capillary temperature 300° C., sheath gas rate (arb) 40, and auxiliary gas rate (arb) 10. Mass spectra were acquired in the full scan (m/z 300-1,500 Da) and PRM acquisition modes at resolving power 70.000 and 35,000 respectively. The LC-MS system was controlled using Xcalibur software. Data was analyzed using Trace Finder software (Thermo Fisher Scientific Inc.).

### Example 1

### Identification of DOX16 and GAME4 Non-Transgenic Mutants Produced by Protoplast Transformation

The inventors aimed to reach modified potato (Solanum tuberosum) plants using protoplasts and CRISPR/Cas9 system. GAME4 and DOX16 and SSR2, were targeted and mutated in the protoplast system.

Leaf pieces were sampled and genomic DNA was isolated from Desiree potato plants that were regenerated from all protoplast transfection experiments. The molecular screen method was PCR amplification of the targeted sequence followed by enzymatic restriction site loss method as described hereinabove. Each site with its specific restriction site (Dox16-Ex1-79-BccI; DOX16-EX2-25-BglII; GAME4-Ex1-155-Van91I; Game4-Ex3-253-Van91I).

Four 4 different protoplasts transfections totally created 56 mutated plants (Table 1).

Transfection using plasmid #4794 (plasmid carrying 35S-Cas9 sequence with 3 RNA guides for DOX16, SEQ ID NO:35), created 6 plants out of 197 screen plants (plants Nos. 48-53).

Transfection using plasmid #4823 (plasmid carrying Ubi-Cas9 sequence and 3 RNA guides for DOX16, SEQ ID NO:36), created 9 plants, out of 30 plants. Seven (7) survived (plants Nos. 54-62).

Transfections using 2 plasmids together: #4823 and #4821 (SEQ ID NO:37) (both plasmids carrying Ubi-Cas9 and 3 guides: 3 guides for DOX16 and 3 guides for GAME4 respectively), created 47 plants out of 573 plants screened. Forty two (42) plants survived (plants Nos. 1-47).

Transfection using Cas9 as protein and RNA guide DOX16-EX2-25 (RNA was prepared from plasmid #4728, SEQ ID NO:38), created 1 plant out of 377 screened plants (plant No. 63).

Since each plasmid carries 3 different guides, potentially every guide can cause a mutation in its selective site. Plasmids #4794 and #4823 have the same three DOX16 RNA guides. The inventors have checked 2 guides activity and decided to work with the one that showed better results: DOX16-Ex1-79.

In transfections of 2 plasmids at the same time, (like #4821 and #4823) six RNA guides were active in the system, mutation at DOX16 gene was checked on site of guide Dox16-Ex1-79, and mutations at GAME4 were checked on 2 sites of guides: Game4-Ex1-155 and Game4-Ex3-253 (the third site wasn't checked, see Table 3). For that reason, plants that came out from these 2 plasmids transfections, can be mutated on both DOX16 and GAME4 genes.

Game4-Ex1-155 RNA guide showed significant activity in potato plant protoplasts. Transfections with this RNA guide revealed in many plants 4 allele mutations. Twenty seven (27) out of 47 potato mutated plants are fully mutated in this site of GAME4. (Table 3-marked as “full knockout”).

In order to evaluate insertions of foreign DNA into the plant genome caused by the transfection processes all 56 mutated plants were checked for Cas9 sequence in their genome. Using PCR, 41% of plant (23 out of 56) did not show Cas9 sequence in their genome and accordingly, are defined herein as “non-transgenic plants” (see Table 3).

TABLE 3
Plant regenerated from transformed protoplasts

| Potato | | Plant | | | | Transgenic |
|---|---|---|---|---|---|---|
| line | Clone | No. | Plant Mutations | | | to Cas9 |
| Desiree | 4821/ | 7 | Dox16 | | | Negative |
| | 4823 | | Ex1-79 | | | |
| Desiree | 4821/ | 8 | Dox16 | | | Positive |
| | 4823 | | Ex1-79 | | | |
| Desiree | 4821/ | 13 | Dox16 | Game 4 Ex1-155 | Game 4 | Positive |
| | 4823 | | Ex1-79 | full knockout | Ex3-253 | |
| Desiree | 4821/ | 14 | Dox16 | Game 4 Ex1-155 | Game 4 | Positive |
| | 4823 | | Ex1-79 | | Ex3-253 | |
| Desiree | 4821/ | 16 | Dox16 | | | Positive |
| | 4823 | | Ex1-79 | | | |
| Desiree | 4821/ | 17 | Dox16 | Game 4 Ex1-155 | Game 4 | Positive |
| | 4823 | | Ex1-79 | | Ex3-253 | |
| Desiree | 4821/ | 18 | Dox16 | | | Negative |
| | 4823 | | Ex1-79 | | | |
| Desiree | 4821/ | 21 | Dox16 | Game 4 Ex1-155 | Game 4 | Positive |
| | 4823 | | Ex1-79 | | Ex3-253 | |
| Desiree | 4821/ | 23 | Dox16 | | | Positive |
| | 4823 | | Ex1-79 | | | |
| Desiree | 4821/ | 62 | Dox16 | | | Negative |
| | 4823 | | Ex1-79 | | | |
| Desiree | 4821/ | 69 | | | Game 4 | Negative |
| | 4823 | | | | Ex3-253 | |
| Desiree | 4821/ | 70 | Dox16 | | | Positive |
| | 4823 | | Ex1-79 | | | |
| Desiree | 4821/ | 71 | | | Game 4 | Negative |
| | 4823 | | | | Ex3-253 | |
| Desiree | 4821/ | 73 | Dox16 | | | Negative |
| | 4823 | | Ex1-79 | | | |
| Desiree | 4821/ | 74 | Dox16 | | | Positive |
| | 4823 | | Ex1-79 | | | |
| Desiree | 4821/ | 75 | | Game 4 Ex1-155 | Game 4 | Positive |
| | 4823 | | | full knockout | Ex3-253 | |
| Desiree | 4821/ | 78 | Dox16 | Game 4 Ex1-155 | Game 4 | Positive |
| | 4823 | | Ex1-79 | full knockout | Ex3-253 | |
| Desiree | 4821/ | 79 | Dox16 | Game 4 Ex1-155 | Game 4 | Positive |
| | 4823 | | Ex1-79 | full knockout | Ex3-253 | |
| Desiree | 4821/ | 80 | Dox16 | Game 4 Ex1-155 | Game 4 | Positive |
| | 4823 | | Ex1-79 | full knockout | Ex3-253 | |
| Desiree | 4821/ | 83 | Dox16 | | | Negative |
| | 4823 | | Ex1-79 | | | |
| Desiree | 4821/ | 84 | Dox16 | Game 4 Ex1-155 | Game 4 | Positive |
| | 4823 | | Ex1-79 | full knockout | Ex3-253 | |
| Desiree | 4821/ | 86 | Dox16 | Game 4 Ex1-155 | Game 4 | Positive |
| | 4823 | | Ex1-79 | full knockout | Ex3-253 | |
| Desiree | 4821/ | 91 | Dox16 | | | Negative |
| | 4823 | | Ex1-79 | | | |
| Desiree | 4821/ | 99 | Dox16 | | | Negative |
| | 4823 | | Ex1-79 | | | |
| Desiree | 4821/ | 103 | Dox16 | | | Negative |
| | 4823 | | Ex1-79 | | | |
| Desiree | 4821/ | 106 | Dox16 | | | Negative |
| | 4823 | | Ex1-79 | | | |
| Desiree | 4821/ | 107 | Dox16 | | | Negative |
| | 4823 | | Ex1-79 | | | |
| Desiree | 4821/ | 113 | Dox16 | Game 4 Ex1-155 | Game 4 | Positive |
| | 4823 | | Ex1-79 | full knockout | Ex3-253 | |
| Desiree | 4821/ | 119 | Dox16 | Game 4 Ex1-155 | Game 4 | Positive |
| | 4823 | | Ex1-79 | full knockout | Ex3-253 | |
| Desiree | 4821/ | 120 | Dox16 | Game 4 Ex1-155 | Game 4 | Positive |
| | 4823 | | Ex1-79 | full knockout | Ex3-253 | |
| Desiree | 4821/ | 141 | | Game 4 Ex1-155 | Game 4 | Positive |
| | 4823 | | | full knockout | Ex3-253 | |
| Desiree | 4821/ | 142 | | Game 4 Ex1-155 | | Negative |
| | 4823 | | | | | |
| Desiree | 4821/ | 148 | | Game 4 Ex1-155 | | Negative |
| | 4823 | | | | | |
| Desiree | 4821/ | 169 | | Game 4 Ex1-155 | | Negative |
| | 4823 | | | | | |
| Desiree | 4821/ | 172 | | Game 4 Ex1-155 | Game 4 | Positive |
| | 4823 | | | full knockout | Ex3-253 | |
| Desiree | 4821/ | 179 | | Game 4 Ex1-155 | Game 4 | Positive |
| | 4823 | | | full knockout | Ex3-253 | |
| Desiree | 4821/ | 201 | | Game 4 Ex1-155 | Game 4 | Positive |
| | 4823 | | | full knockout | Ex3-253 | |
| Desiree | 4821/ | 209 | | Game 4 Ex1-155 | Game 4 | Positive |
| | 4823 | | | full knockout | Ex3-253 | |
| Desiree | 4821/ | 210 | | Game 4 Ex1-155 | Game 4 | Positive |
| | 4823 | | | full knockout | Ex3-253 | |
| Desiree | 4821/ | 249 | | Game 4 Ex1-155 | | Positive |
| | 4823 | | | full knockout | | |
| Desiree | 4821/ | 260 | | Game 4 Ex1-155 | | Positive |
| | 4823 | | | full knockout | | |
| Desiree | 4821/ | 262 | | Game 4 Ex1-155 | | Positive |
| | 4823 | | | full knockout | | |
| Desiree | 4821/ | 263 | | Game 4 Ex1-155 | | Positive |
| | 4823 | | | full knockout | | |
| Desiree | 4821/ | 293 | | Game 4 Ex1-155 | | Positive |
| | 4823 | | | full knockout | | |
| Desiree | 4821/ | 356 | | Game 4 Ex1-155 | | Positive |
| | 4823 | | | full knockout | | |
| Desiree | 4821/ | 394 | | Game 4 Ex1-155 | | Positive |
| | 4823 | | | full knockout | | |
| Desiree | 4821/ | 395 | | Game 4 Ex1-155 | | Positive |
| | 4823 | | | full knockout | | |
| Desiree | 4794 | 29 | Dox16 | | | Negative |
| | | | Ex1-79 | | | |
| Desiree | 4794 | 30 | Dox16 | | | Negative |
| | | | Ex1-79 | | | |
| Desiree | 4794 | 33 | Dox16 | | | Negative |
| | | | Ex1-79 | | | |
| Desiree | 4794 | 75 | Dox16 | | | Negative |
| | | | Ex1-79 | | | |
| Desiree | 4794 | 113 | Dox16 | | | Negative |
| | | | Ex1-79 | | | |
| Desiree | 4794 | 64 | Dox16 | | | Negative |
| | | | Ex1-79 | | | |
| Desiree | 4823 | 2 | Dox16 | | | Negative |
| | | | Ex1-79 | | | |
| Desiree | 4823 | 3 | Dox16 | | | Negative |
| | | | Ex1-79 | | | |
| Desiree | 4823 | 6 | Dox16 | | | Negative |
| | | | Ex1-79 | | | |
| Desiree | 4823 | 7 | Dox16 | | | ? |
| | | | Ex1-79 | | | |
| Desiree | 4823 | 10 | Dox16 | | | Negative |
| | | | Ex1-79 | | | |
| Desiree | 4823 | 11 | Dox16 | | | Negative |
| | | | Ex1-79 | | | |
| Desiree | 4823 | 13 | Dox16 | | | Positive |
| | | | Ex1-79 | | | |
| Desiree | 4823 | 14 | Dox16 | | | Negative |
| | | | Ex1-79 | | | |
| Desiree | 4823 | 24 | Dox16 | | | Negative |
| | | | Ex1-79 | | | |
| Desiree | 4728 | 65 | Dox16 | | | Negative |
| | | | Ex2-25 | | | |

### Example 2

### Identification of DOX16 Transgenic Mutant Potato Plants

The inventors aimed at creating mutated potato plants using the CRISPR/Cas9 system. Two target genes were chosen DOX16 and SSR2. The current strategy was first, to create transgenic commercial potato lines that constitutively express the Cas9 gene and second, to deliver the guide RNA's for targeted mutagenesis into those lines by the MEMOGENE™ TRV based viral system. The combination of Cas9 constant expression and temporary guide RNA expression were expected to yield mutated plants, regenerating in tissue culture conditions.

### A. Production of DOX16 Mutant Plants

Leaf pieces were sampled, and genomic DNA was isolated from Desiree and Pfifer potato plants that were regenerated from all viral inoculation experiments. The molecular screen method was PCR amplification of the targeted sequence followed by enzymatic restriction site-loss method using BglII-FD enzyme (Thermo scientific). Out of 202 screened Desiree plants, the inventors detected 6 plants with Dox16 mutation and out of 46 screened Pfifer plants, 1 plant with Dox16 mutation was detected. All 7 mutated plants had both cut and uncut bands, suggesting that some, but not all, alleles were mutated.

The uncut band in those 7 specific mutant plants were cloned into pGEM-T-EASY plasmids (Clontech) and used to create E. coli libraries. Ten (10) clones of each library were sent to Sanger sequence to identify the indel identity in each line. All clones in each library had the same sequence results, suggesting that all mutants were mutated only in one of four alleles of the gene. The sequencing results are summarized in Table 4.

TABLE 4
DOX16 mutant plants produced by the CRISPR/Cas9 system

| | Mutated | | | |
|---|---|---|---|---|
| | DOX16 | Mutation | | Missing sequence |
| Plant No. | alleles | identity | | (indel) |
| 43 | 1 | −4 | bp | −TCTA |
| 62 | 1 | −1 | bp | −T |
| 71 | 1 | −7 | bp | −TCTAATG |
| 259 | 1 | −12 | bp | −TCTAATGGATGA |
| 271 | 1 | +1 | bp | +A |
| 283 | 1 | −3 | bp | −TCT |
| 21 (The only pfifer plant) | 1 | −4 | bp | −TCTA |

To eliminate the possibility of chimeras, the inventors took the shoot tip of each of the mutants and re-rooted it aside of the original plant. Then, leaves from 2 distinct parts of the 2 “replicas” were sampled and analyzed as above so as to examine the presence and identity of mutated sequences. In all cases, same mutations were found in the different samplings and no signs of chimerism were detected. Moreover, all plants appeared perfectly normal, and indistinguishable from the wild type clones of Desiree and Pfifer.

Following in vitro propagation of those mutants, they were moved to the greenhouse for SGA analysis and tuber production.

### B. Production of SSR2 Mutant Plants

Leaf pieces were sampled and genomic DNA was isolated from Desiree potato plants that were regenerated from viral inoculation experiments. The molecular screen method was PCR amplification of the targeted sequence followed by enzymatic restriction site-loss method using BseNI-FD enzyme (Thermo scientific). Out of 100 screened Desiree plants, the inventors detected 9 plants with SSR2 insertion/deletion (indel).

All 9 mutated plants had both cut and uncut bands, suggesting that some, but not all of the alleles was mutated. The uncut bands in 4 of those specific mutant plants were cloned into pGEM-T-EASY plasmid (Clontech) and used to create E. coli libraries. Ten (10) clones of each library were sent to Sanger sequence to identify the indel identity in each line. In two libraries all clones had the same sequence results, suggesting that all mutants were mutated only in one of four alleles of the gene, but in the other 2 libraries, the inventors received 2 different indel sequences, suggesting that 2 alleles were mutated in those plants. The sequencing results are summarized in table 5:

TABLE 5
SSR2 mutant plants produced by the CRISPR/Cas9 system

| | Mutated | | |
|---|---|---|---|
| | DOX16 | | Missing sequence |
| Plant No. | alleles | Mutation identity | (indel) |
| 7 | 1 | +1 bp | +A |
| 8 | 1 | −1 bp | −C |
| 19 | | | |
| 63 | 2 | −1 bp and −79 bp + 9 bp | −C and −70 bp indel |
| 65 | | | |
| 72 | 2 | −1 bp | −C, −C |

Following in vitro propagation of those mutants, they were moved to the greenhouse for SGA analysis and tuber production.

### Example 3

SGA Content in Haulm of Potato Plants Regenerated from Mutated Protoplasts

Content of the SGAs α-chaconine and α-solanine in the haulm of the plants described in Table 3 was determined as detailed in the Method section hereinabove. Table 6 below presents the SGAs content in haulm of exemplary mutated plant lines (average of 2-4 plants), used for the digestibility experiment described in Example 4 hereinbelow.

TABLE 6
SGA content in haulm of mutated potato plants

| | | Steroidal Glycoalkaloids | |
|---|---|---|---|
| Plant | | (SGAs) μg/Dry Weight | |
| No. | Mutation | α-chaconine | α-solanine |
| 8 | Dox16 Ex1-79 | 1.025 | 0.70 |
| 119 | Dox16 Ex1-79; | 1.013 | 0.50 |
| | Game 4 Ex1-155 full knockout; | | |
| | Game 4 Ex3-253 | | |
| 172 | Game 4 Ex1-155 full knockout; | 0.60 | 0.28 |
| | Game 4 Ex3-253 | | |
| 209 | Game 4 Ex1-155 full knockout; | 0.44 | 0.21 |
| | Game 4 Ex3-253 | | |
| 293 | Game 4 Ex1-155 full knockout | 0.38 | 0.18 |
| 395 | Game 4 Ex1-155 full knockout | 0.38 | 0.18 |
| WT | | 5,259-15,551 | 1,502-6,257 |

As clearly can be concluded from Table 6, the content of α-chaconine and α-solanine in the haulm of plants of the current invention is significantly reduced compared to wild type and is negligible, thus rendering the haulm derived therefrom as an edible composition, such as for use as a feed for ruminant farmed animals.

### Example 4

### Digestibly of Potato Haulm in Comparison to Prominent Ruminant Feedstuff

A preliminary step for trials testing ruminant feeding is the examination of the feedstuff of interest using in vitro digestion methods.

The digestibility of commonly used ruminant feedstuff including grains (barely, corn, wheat), residues of human food industry (potato) and silage (legumes, barley, corn and wheat) was examined using in vitro digestibility method.

For this purpose, all feed types were lyophilized in order to prevent biochemical changes in the feed's nutritional values and properties. All feeds were ground in a Wiley mill (Arthur H. Thomas, Philadelphia, PA) to pass a 1-mm pore-size screen. Rumen digesta was collected before the morning meal from the reticulum near the reticuloomasal orifice of the sheep by a vacuum pump. The digesta was filtered through eight layers of gauze cloth, mixed on a volume basis for each species, purged with CO₂, and kept in a prewarmed thermos until use (within approximately 20 min). The incubation inoculum was prepared by diluting the digesta inoculum with the buffer according to the method described by Tilly and Terry (1963. Grass Forage Sci., 18 (2): 104-111), in a 1:4 (vol/vol) ratio and stirring in a water bath at 39° C. with purging CO₂ until its use (10 to 15 min later). The digesta inoculum was filtered once more and added to the feed sample at a standard concentration. All samples were incubated for 48 h at 39° C. Each sample was incubated in six replicates for each source of inoculum at two different occasions (runs; at least 2 weeks apart). At the end of the 48-h incubation period, contents were acidified by adding 6 M HCl to reach a final pH of 1.3 to 1.5. After a few seconds, when the foam subsided, pepsin powder (EC 3.4.23.1) was added to a final concentration of 0.2% (weight/vol). The samples were re-incubated for an additional 48-h. At the end of this stage, the samples were rinsed thoroughly with tap water until the rinse was clear. The tubes were centrifuged at 2,500×g for 15 min, and the supernatant was discarded. To the pellet, 50 ml of H₂O was added and the tubes were recentrifuged to wash out the residual acid. The tubes containing the pellets and the bags were dried in a forced air oven at 60° C. for 48-h to determine the residual dry matter (DM) weights. In vitro dry matter digestibility (IVDMD) was calculated as the DM which disappeared from the initial weight.

Microflora mass from buffered non-supplemented fermented ruminants' digestive system was measured and served as blank.

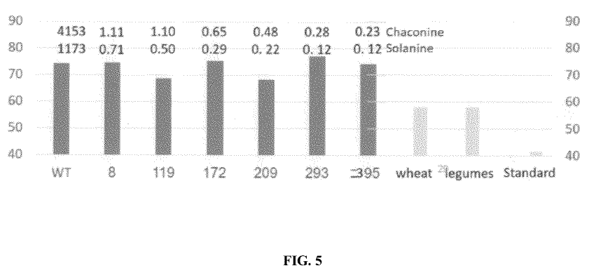

In vitro digestibility measurements successfully simulated the digestion process in ruminants' abdomen. Digestion of standard silage was found to be about half the digestibility of grains and residues from the human food industry (FIG. 5).

Next, the digestibility of potato haulm of the herein disclosed low-SGA mutant lines, presented in Table 4 hereinabove, was examined in comparison to commonly used hays of legumes and wheat. As is apparent in FIG. 5, the digestibility of all the low-SGA mutant lines haulm ranged from about 70% to about 80%, depending on the mutant, similar to the digestibility of non-mutated potato haulm (WT), while the digestion of the commonly used hays ranges from about 50% to about 60%.

The very mutation that eliminated SGAs production did not impair the potato haulm digestibility compared to the control of the source plant.

Summarized herein below in Tables 7-10, are chemical content, mineral content, anion content, and amino acids content analyses, respectively, of genetically modified plants as disclosed herein.

TABLE 7
Chemical content analysis

| | | % | % | % | % | % | % | | | |
|---|---|---|---|---|---|---|---|---|---|---|
| Genotype | DM | Ash | EE | CP | Starch | NDF | ADF | IVDMD | IVNDFD | IVNDF48 |
| 8 | 11.13 | 8.81 | 2.31 | 40.1 | 0.55 | 34.8 | 15.6 | 67.5 | 67.3 | 84.4 |
| 16 | 5.92 | 5.25 | 1.50 | 45.3 | 3.51 | 38.2 | 14.0 | 73.2 | 77.1 | 88.5 |
| 103 | 10.70 | 5.58 | 1.96 | 40.0 | ND | 37.9 | 21.0 | 59.4 | 48.2 | 73.0 |
| 119 | 8.21 | 9.35 | 2.42 | 38.8 | ND | 37.8 | 16.8 | 69.4 | 73.7 | 86.5 |
| 172 | 5.08 | 12.60 | 1.33 | 44.8 | ND | 38.3 | 17.4 | 66.2 | 64.8 | 87.3 |
| 395 | 5.44 | 13.61 | 1.70 | 44.3 | ND | 31.2 | 16.0 | 64.1 | 52.9 | 85.7 |
| WT | 12.77 | 5.93 | 3.12 | 31.5 | ND | 46.6 | 23.4 | 60.7 | 66.5 | 77.9 |

TABLE 8
Minerals content analysis

| | %, on DM basis | | | | | | mg/kg DM | | | | |
|---|---|---|---|---|---|---|---|---|---|---|---|
| Genotype | Ca | P | Mg | K | Na | S | Cu | Fe | Mn | Ag | Se |
| 8 | 0.90 | 0.49 | 0.38 | 3.92 | 0.08 | 0.35 | 5.16 | 304.68 | 54.06 | <0.86 | <0.70 |
| 16 | 0.94 | 0.67 | 0.41 | 5.18 | 0.01 | 0.38 | 4.54 | 130.38 | 55.39 | <0.84 | <0.69 |
| 103 | 1.13 | 0.46 | 0.45 | 3.28 | 0.10 | 0.33 | 3.93 | 201.85 | 78.27 | <0.83 | <0.67 |
| 119 | 0.80 | 0.48 | 0.31 | 4.77 | 0.09 | 0.37 | 5.18 | 125.46 | 55.72 | <0.84 | <0.68 |
| 172 | 0.72 | 0.66 | 0.26 | 7.28 | 0.01 | 0.36 | 4.88 | 105.87 | 37.00 | <0.84 | <0.69 |
| 395 | 1.00 | 0.75 | 0.29 | 7.86 | 0.02 | 0.40 | 4.65 | 133.66 | 86.03 | <0.83 | <0.68 |
| WT | 1.63 | 0.22 | 0.38 | 1.17 | 0.12 | 0.28 | 4.08 | 224.26 | 68.13 | <0.84 | <0.68 |

TABLE 9
Anions content analysis

| Geno | mg/kg DM | | | | |
|---|---|---|---|---|---|
| type | Chloride | Sulphate | Phosphate | Nitrite | Nitrate |
| 8 | 1.42 | 0.30 | 0.63 | ND | 4.91 |
| 16 | 1.42 | 0.38 | 1.20 | ND | 8.80 |
| 103 | 1.74 | 0.38 | 0.77 | ND | 7.57 |
| 119 | 1.35 | 0.42 | 0.71 | ND | 5.19 |
| 172 | 1.51 | 0.47 | 1.30 | ND | 12.0 |
| 395 | 1.22 | 0.67 | 1.43 | ND | 14.3 |
| WT | 1.51 | 0.21 | 0.27 | ND | 1.46 |

TABLE 10
Amino acids content analysis

| | AA - mg/g DM | | | | | | | | | | | | | | | | |
|---|---|---|---|---|---|---|---|---|---|---|---|---|---|---|---|---|---|
| | A | R | D | E | Q | H | H-P | L | I | K | M | F | P | S | T | Y | V |
| 8 | 15.2 | 11.8 | 24.3 | 35.8 | 10.2 | 5.43 | 1.28 | 18.6 | 9.49 | 12.7 | 1.58 | 11.9 | 9.89 | 9.30 | 9.71 | 6.67 | 11.6 |
| 16 | 17.7 | 14.4 | 27.0 | 42.9 | 12.5 | 5.91 | 1.31 | 22.9 | 11.7 | 15.1 | 2.50 | 14.7 | 11.9 | 10.2 | 11.3 | 7.45 | 14.0 |
| 103 | 13.4 | 11.0 | 35.1 | 42.7 | 9.21 | 4.93 | 1.71 | 18.4 | 9.95 | 12.0 | 1.63 | 12.2 | 12.0 | 9.1 | 9.79 | 6.39 | 12.6 |
| 119 | 17.3 | 14.5 | 26.4 | 36.1 | 11.9 | 6.33 | 1.49 | 23.0 | 11.1 | 14.0 | 3.21 | 14.3 | 11.5 | 10.7 | 11.4 | 9.85 | 13.7 |
| 172 | 14.7 | 11.8 | 25.3 | 36.6 | 10.2 | 4.23 | 1.15 | 19.6 | 9.86 | 13.0 | 1.10 | 12.5 | 10.0 | 8.41 | 7.92 | 2.37 | 11.7 |
| 395 | 18.8 | 14.9 | 24.0 | 34.3 | 13.9 | 6.08 | 1.63 | 25.8 | 12.5 | 17.8 | 2.24 | 15.8 | 13.3 | 9.96 | 11.1 | 7.61 | 15.1 |
| WT | 9.56 | 8.38 | 18.3 | 24.1 | 7.96 | 3.73 | 1.47 | 13.3 | 6.70 | 8.38 | 2.33 | 8.44 | 7.75 | 6.71 | 7.08 | 7.54 | 8.76 |

The inventors have subsequently performed a next generation sequencing (NGS) analysis for the mutated GAME4 enzyme encoding gene. The results showed that in lines 103, 65 and 142, there was an amplicon at the same size as the WT plant. In sharp contrast, when testing all the other mutant lines (8, 16,119,141, 175, 262, & 395) amplicon similar to the WT was not detected at all. Therefore, strongly suggesting that all these mutant lines were fully mutated for GAME4, e.g., all alleles of the genome were deleterious for GAME4 expression and/or activity.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. An edible composition comprising a plant belonging to the genus Solanum or plant part derived therefrom, wherein the plant or plant part is essentially devoid of steroidal glycoalkaloids (SGAs), and wherein the in vitro dry matter digestibility (IVDMD) of said plant or plant part is greater than or essentially equivalent to the IVDMD of a control plant belonging to the genus Solanum or plant part comprising SGAs.

2. The edible composition of claim 1, wherein said plant or plant part comprises a protein content of 35-50% weight per weight of dry matter of said plant or plant part.

3. The edible composition of claim 1, wherein said plant or plant part comprises potassium in an amount of 3-10% weight per weight of dry matter of said plant or plant part.

4. The edible composition of claim 1, wherein said plant or plant part comprises at least one essential amino acid being selected from the group consisting of: Histidine, Isoleucine, Leucine, Lysine, Phenylalanine, Threonine, Valine, and any combination thereof, in an amount being at least 10% by weight greater that in a control plant.

5. The edible composition of claim 1, wherein said plant belonging to the genus Solanum is selected from the group consisting of: potato, tomato, and eggplant.

6. The edible composition of claim 1, wherein said plant part comprises haulm.

7. The edible composition of claim 6, wherein said haulm comprises from 0 to 0.25 mg SGAs per 100 g fresh weight (FW).

8. The edible composition of claim 6, wherein said IVDMD of said haulm is greater than or essentially equivalent to the IVDMD of a standard hay or silage feed.

9. The edible composition of claim 1, wherein said plant or plant part is genetically modified such that at least one enzyme being selected from the group consisting of: 2-oxoglutarate-dependent dioxygenase (16DOX), glycoalkaloid metabolism 4 (GAME4), and both, is characterized by having reduced expression and/or activity compared to the expression and/or activity of said at least one enzyme in a control plant or plant part.

10. The edible composition of claim 8, wherein said genetically modified plant or plant part comprises a mutation in Exon 1 of a gene encoding said DOX16 enzyme.

11. The edible composition of claim 8, wherein said genetically modified plant or plant part comprises a mutation in Exon 1, Exon 3, or both, of a gene encoding said GAME4 enzyme.

12. The edible composition of claim 10 or 11, wherein said mutation comprises a deletion or insertion.

13. The edible composition of claim 12, wherein any one of said insertion and deletion is of 1 to 15 nucleotides.

14. The edible composition of claim 10, wherein said mutation is introduced into an exon of a gene using a programmable engineered nuclease (PEN).

15. The edible composition of claim 14, wherein said PEN is a clustered regularly interspaced short palindromic repeat (CRISPR) type II system a gene-editing method.

16. The edible composition of claim 1, wherein said plant or plant part is devoid of heterologous polynucleotides.

17. The edible composition of 1, wherein said control plant comprises a wild type (WT) plant belonging to the genus Solanum.

18.-20. (canceled)

21. A genetically modified plant belonging to the genus Solanum characterized by being essentially devoid of SGAs and having IVDMD greater than or essentially equivalent to the IVDMD of a control plant, the genetically modified plant comprising at least one enzyme being selected from the group consisting of: 16DOX, GAME4, and both, being characterized by having reduced expression and/or activity compared to the expression and/or activity of said at least one enzyme in a control plant.

22. A method for producing a plant or plant part being essentially devoid of SGAs, and having an IVDMD being greater than or essentially equivalent to the IVDMD of a control plant, the method comprising mutating at least one gene encoding at least one enzyme being selected from the group consisting of: 16DOX, GAME4, and both, such that said at least one enzyme is characterized by having reduced expression and/or activity compared to the expression and/or activity of said at least one enzyme in a control plant or plant part.
